(19) United States
(12) Patent Application Publication
(10) Pub. No.: US 2021/0260130 A1
(43) Pub. Date: Aug. 26, 2021

(54) COMPOSITIONS AND METHODS FOR INHIBITION OF LINEAGE SPECIFIC ANTIGENS

(71) Applicant: The Trustees of Columbia University in the City of New York, New York, NY (US)

(72) Inventors: Siddhartha MUKHERJEE, New York, NY (US); Florence BOROT, New York, NY (US); Abdullah Mahmood ALI, New York, NY (US)

(21) Appl. No.: 17/244,136

(22) Filed: Apr. 29, 2021

Related U.S. Application Data
(63) Continuation of application No. PCT/US2020/013887, filed on Jan. 16, 2020.
(60) Provisional application No. 62/793,210, filed on Jan. 16, 2019.
(60) Provisional application No. 62/852,573, filed on May 24, 2019.

(51) Int. Cl. A61K 35/28; C12N 9/22; C12N 15/113; C07K 14/705

(52) U.S. Cl. CPC A61K 35/28; C12N 9/22; C12N 2310/20; C07K 14/70596; C12N 15/113

(57) ABSTRACT

Disclosed herein are methods of administering an agent targeting a lineage-specific cell-surface antigen, e.g., CD33, and a population of hematopoietic cells that are deficient in the lineage-specific cell-surface antigen, e.g., CD33 for immunotherapy of hematological malignancies. Cells comprising mutations in CD33 are also provided, as are gRNAs targeting CD33.

This application is a continuation of PCT/US2020/013887, filed January 16, 2020, which claims priority to U.S. Ser. No.: 62/793,210, filed Jan. 16, 2019 and U.S. Ser. No.: 62/852,573, filed May 24, 2019, all of which are incorporated by reference, as if expressly set forth in their respective entireties herein.

### SEQUENCE LISTING

The instant application contains a Sequence Listing which has been submitted electronically in ASCII format and is hereby incorporated by reference in its entirety. Said ASCII copy, created on Jan. 16, 2020, is named 01001-004965-WO1_SL.txt and is 83 kilobytes in size.

### BACKGROUND OF DISCLOSURE

Despite decades of attempts, curative immunological therapy against cancer has been very difficult to achieve, with the fundamental basis being antigen-recognition capacity, either by antibodies or through T cells (via the T cell receptor) (Cousin-Frankel, Science (2013) 342:1432). Antibody-based immunotherapies have been used extensively against cancer in instances where the target antigen is up-regulated in tumor cells as compared to normal cells (e.g., Her-2 in Her-2 amplified breast cancer), or in cases where the tumor cells express an antigen that can be recognized by the antibody or an antibody-toxin conjugate (e.g., Rituximab against CD20) (Baselga et al., Annals Oncology (2001) 12:S35). While clinical trials using antibody-based immunotherapies have shown improved patient survival in a limited number of cancer types (usually when combined with standard chemotherapy), these effects are often accompanied by significant safety and efficacy concerns (Cousin-Frankel Cancer, Science (2013) 342:1432).

Effective T cell therapies against cancers have been even more difficult to achieve clinically (Schmitt et al., Hum. Gene Ther. (2009) 20(11):1240). An effective T cell therapy against cancer relies on a T cell with a high affinity binding directed against an antigen on a cancer cell. Chimeric antigen receptor T cells (CAR T cells) are widely used to recognize antigens on cells with both high affinity and specificity and without the requirement for accessory recognition molecules, such as HLA antigens to “present” peptides. The T cell receptor of a CAR T cells is “swapped” with an antigen-binding heavy and light chains, thereby obviating the need for HLA accessory molecules. The recombinant CAR T receptor is fused to signaling domains leading to activation of the T cell upon binding of the CAR T receptor to the target antigen.

The clinical use of CAR T cells has been limited to targeting a narrow range of cell surface antigens, further supporting the need for improved and novel approaches in the treatment of cancer. In particular, new approaches are needed for diseases such as acute myeloid leukemia (AML) in which the outcomes in older patients who are unable to receive intensive chemotherapy, the current standard of care, remains very poor, with a median survival of only 5 to 10 months (Dohner et al., NEJM (2015) 373:1136).

Described herein are novel approaches to cancer immunotherapy that targets certain classes of lineage-specific cell-surface antigens on tumor cells. The CAR T cell treatment is then combined with replacement of the non-tumor cells by infusion or reinfusion of a modified population of cells that are deficient for the lineage-specific cell-surface antigen. Recurrence of the tumor is prevented or decreased by maintaining surveillance of the patient in vivo with the CAR T cells.

### SUMMARY OF DISCLOSURE

The present disclosure is based, at least in part, on the discovery that agents comprising an antigen-binding fragment that binds a lineage-specific cell-surface antigen (e.g., immune cells expressing a chimeric receptor that targets CD33) selectively cause cell death of cells expressing the lineage-specific cell-surface antigen, whereas cells that are deficient for the antigen (e.g., genetically engineered hematopoietic cells) evade cell death caused thereby. Based on such findings, it would have been expected that immunotherapies involving the combination of an agent targeting a lineage-specific cell-surface antigen, for example, CAR-T cells targeting CD33, and hematopoietic cells that are deficient in the lineage-specific cell-surface antigens (e.g., CD33) would provide an efficacious method of treatment for hematopoietic malignancies.

In some aspects, the disclosure provides a genetically engineered hematopoietic stem or progenitor cell, which comprises a genetic mutation in the exon 3 of an endogenous CD33 gene, wherein the genetic mutation is at a site described herein. One aspect of the present disclosure provides a genetically engineered hematopoietic stem and/or progenitor cell, which comprises a genetic mutation in exon 3 of an endogenous CD33 gene, wherein the genetic mutation is at a site targeted by a gRNA, which comprises the nucleotide sequence of AUCCCUGGCACUCUAGAACC (SEQ ID NO: 67), GGCCGGGUUCUAGAGUGCCA (SEQ ID NO: 68), or CCUCACUAGACUUGACCCAC (SEQ ID NO: 70), and wherein the genetically engineered hematopoietic stem and/or progenitor cell has a reduced expression level of CD33 as compared with a wildtype counterpart. In some embodiments, the genetically engineered hematopoietic stem and/or progenitor cell expresses less than 10% of the CD33 expressed by the wild-type counterpart. In some embodiments, the genetically engineered hematopoietic stem and/or progenitor cell does not express CD33. In some embodiments, the genetically engineered hematopoietic stem and/or progenitor cell is CD34⁺. In some embodiments, the genetically engineered hematopoietic stem and/or progenitor cell is from bone marrow cells or peripheral blood mononuclear cells of a subject (e.g., a human patient having a hematopoietic malignancy, or a healthy donor).

The disclosure, in some embodiments, also provides a cell population comprising a plurality of the genetically engineered hematopoietic stem and/or progenitor cells described herein.

In another aspect, the present disclosure provides a method of producing a genetically engineered hematopoietic stem and/or progenitor cell, comprising (i) providing a hematopoietic stem and/or progenitor cell, and (ii) introducing into the cell (a) a guide RNA (gRNA) that comprises a nucleotide sequence that is at least 90% identical to SEQ ID NO: 67, SEQ ID NO: 68, and/or SEQ ID NO: 70, and (b) a Cas9 endonuclease, thereby producing a genetically engineered hematopoietic stem and/or progenitor cell having a reduced expression level of CD33. In some embodiments, the gRNA and Cas9 endonuclease are encoded on one vector, which is introduced into the cell. In some embodiments, the vector is a viral vector. In some embodiments, the gRNA and Cas9 endonuclease are introduced into the cell as a pre-formed ribonucleoprotein complex. In some embodiments, the ribonucleoprotein complex is introduced into the cell via electroporation.

The present disclosure also provides, in some aspects, use of a gRNA described herein for reducing expression of CD33 in a sample of hematopoietic cells stem or progenitor cells using a CRISPR/Cas9 system.

The present disclosure also provides, in some aspects, use of a CRISPR/Cas9 system for reducing expression of CD33 in a sample of hematopoietic cells stem or progenitor cells, wherein the gRNA of the CRISPR/Cas9 system is a gRNA described herein.

In some embodiments the gRNA is a single-molecule guide RNA (sgRNA). In some embodiments, the gRNA is a modified sgRNA. In some embodiments, the hematopoietic stem and/or progenitor cell is CD34⁺. In some embodiments, the hematopoietic stem and/or progenitor cell is from bone marrow cells or peripheral blood mononuclear cells (PBMCs) of a subject. In some embodiments, the subject has a hematopoietic disorder. In some embodiments, the subject is a healthy HLA-matched donor.

The disclosure, in some embodiments, provides a genetically engineered hematopoietic stem and/or progenitor cell, which is produced by a method described herein.

In another aspect, the present disclosure provides a method of treating a hematopoietic disorder, comprising administering to a subject in need thereof an effective amount of the genetically engineered hematopoietic stem and/or progenitor cell or the cell population described herein. In some embodiments, the hematopoietic disorder is a hematopoietic malignancy.

In some embodiments, the method further comprises administering to the subject an effective amount of an agent that targets CD33, wherein the agent comprises an antigen-binding fragment that binds CD33. In some embodiments, the agent that targets CD33 is an immune cell expressing a chimeric antigen receptor (CAR), which comprises the antigen-binding fragment that binds CD33.

In some aspects, the present disclosure provides a genetically engineered hematopoietic stem or progenitor cell described herein or a cell population described herein for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33.

In some aspects, the present disclosure provides an agent that targets CD33, wherein the agent comprises an antigen-binding fragment that binds CD33, for use in treating a hematopoietic disorder, wherein the treating comprises administering to a subject in need thereof an effective amount of the agent that targets CD33, and further comprises administering to the subject an effective amount of a genetically engineered hematopoietic stem or progenitor cell described herein or a cell population described herein.

A combination of a genetically engineered hematopoietic stem or progenitor cell described herein or a cell population described herein, and an agent that targets CD33, wherein the agent comprises an antigen-binding fragment that binds CD33, for use in treating a hematopoietic disorder, wherein the treating comprises administering to a patient in need thereof an effective amount of the genetically engineered hematopoietic stem or progenitor cell or the cell population, and the agent that binds CD33.

In some embodiments, the genetically engineered hematopoietic stem or progenitor cell or the cell population is administered concomitantly with the agent that targets CD33. In some embodiments, the genetically engineered hematopoietic stem or progenitor cell or the cell population is administered prior to the agent that targets CD33. In some embodiments, the agent that targets CD33 is administered prior to the genetically engineered hematopoietic stem or progenitor cell or the cell population.

In some embodiments, the immune cell is a T cell. In some embodiments, the immune cells, the genetically engineered hematopoietic stem and/or progenitor cell, or both, are allogeneic. In some embodiments, the immune cells, the genetically engineered hematopoietic stem and/or progenitor cell, or both, are autologous. In some embodiments, the antigen-binding fragment in the chimeric receptor is a single-chain antibody fragment (scFv) that specifically binds human CD33.

In some embodiments, the subject is a human patient having Hodgkin's lymphoma, non-Hodgkin's lymphoma, leukemia, or multiple myeloma. In some embodiments, the subject is a human patient having leukemia, which is acute myeloid leukemia, chronic myelogenous leukemia, acute lymphoblastic leukemia, or chronic lymphoblastic leukemia.

The disclosure, in another aspect, provides a guide ribonucleic acid (gRNA) comprising a spacer sequence that is at least 90% identical to SEQ ID NO: 67, SEQ ID NO: 68, or SEQ ID NO: 70. In some embodiments, the gRNA is a single-molecule gRNA (sgRNA). In some embodiments, the gRNA is modified. In specific examples, the spacer sequence is SEQ ID NO:67, SEQ ID NO:68, or SEQ ID NO:70.

Features of the compositions and methods herein are also described in the following enumerated embodiments.
1. A genetically engineered hematopoietic stem or progenitor cell, which comprises a genetic mutation in the exon 3 of an endogenous CD33 gene, wherein the genetic mutation is at a site having a sequence chosen from:

(SEQ ID NO: 50)
ATCCCTGGCACTCTAGAACC,
(SEQ ID NO: 49)
ATCCCTGGCACTCTAGAACCCGG,
(SEQ ID NO: 51)
GGCCGGGTTCTAGAGTGCCA,
(SEQ ID NO: 29)
GGCCGGGTTCTAGAGTGCCAGGG,
(SEQ ID NO: 58)
CCTCACTAGACTTGACCCAC;
or
(SEQ ID NO: 48)
CCTCACTAGACTTGACCCACAGG.

2. A genetically engineered hematopoietic stem or progenitor cell, which comprises a genetic mutation in the exon 3 of an endogenous CD33 gene, wherein the genetic mutation is at a site having a sequence of ATCCCTGGCACTCTAGAACC (SEQ ID NO: 50).
3. A genetically engineered hematopoietic stem or progenitor cell, which comprises a genetic mutation in the exon 3 of an endogenous CD33 gene, wherein the genetic mutation is at a site having a sequence of ATCCCTGGCACTCTAGAACC (SEQ ID NO: 50), wherein the genetic mutation results in a reduced expression level of CD33 as compared with a wild-type counterpart cell.
4. A genetically engineered hematopoietic stem or progenitor cell, which comprises a genetic mutation in the exon 3 of an endogenous CD33 gene, wherein the genetic mutation is at a site having a sequence of ATCCCTGGCACTCTAGAACC (SEQ ID NO: 50), wherein the genetic mutation results in a reduced expression level of CD33 that is less than 20% of the level of CD33 in a wild-type counterpart cell.
5. A genetically engineered hematopoietic stem or progenitor cell, which comprises a genetic mutation in the exon 3 of an endogenous CD33 gene, wherein the genetic mutation is at a site having a sequence of ATCCCTGGCACTCTAGAACCCGG (SEQ ID NO: 49).
6. A genetically engineered hematopoietic stem or progenitor cell, which comprises a genetic mutation in the exon 3 of an endogenous CD33 gene, wherein the genetic mutation is at a site having a sequence of CCTCACTAGACTTGACCCAC (SEQ ID NO: 58).
7. A genetically engineered hematopoietic stem or progenitor cell, which comprises a genetic mutation in the exon 3 of an endogenous CD33 gene, wherein the genetic mutation is at a site having a sequence of CCTCACTAGACTTGACCCAC (SEQ ID NO: 58), wherein the genetic mutation results in a reduced expression level of CD33 as compared with a wild-type counterpart cell.
8. A genetically engineered hematopoietic stem or progenitor cell, which comprises a genetic mutation in the exon 3 of an endogenous CD33 gene, wherein the genetic mutation is at a site having a sequence of CCTCACTAGACTTGACCCAC (SEQ ID NO: 58), wherein the genetic mutation results in a reduced expression level of CD33 that is less than 20% of the level of CD33 in a wild-type counterpart cell.
9. A genetically engineered hematopoietic stem or progenitor cell, which comprises a genetic mutation in the exon 3 of an endogenous CD33 gene, wherein the genetic mutation is at a site having a sequence of CCTCACTAGACTTGACCCACAGG (SEQ ID NO: 48).
10. A genetically engineered hematopoietic stem or progenitor cell, which comprises a genetic mutation in the exon 3 of an endogenous CD33 gene, wherein the genetic mutation is at a site targeted by a gRNA, which comprises the nucleotide sequence of AUCCCUGGCACUCUAGAACC (SEQ ID NO: 67) or CCUCACUAGACUUGACCCAC (SEQ ID NO: 70), and wherein the genetically engineered hematopoietic stem and/or progenitor cell has a reduced expression level of CD33 as compared with a wild-type counterpart.
11. A genetically engineered hematopoietic stem or progenitor cell, which comprises a genetic mutation in the exon 3 of an endogenous CD33 gene, wherein the genetic mutation is at a site targeted by a gRNA which comprises the nucleotide sequence of

(SEQ ID NO: 70)
CCUCACUAGACUUGACCCAC.

12. A genetically engineered hematopoietic stem or progenitor cell, which comprises a genetic mutation in the exon 3 of an endogenous CD33 gene, wherein the genetic mutation is at a site targeted by a gRNA which comprises the nucleotide sequence of

(SEQ ID NO: 67)
AUCCCUGGCACUCUAGAACC.

13. The genetically engineered hematopoietic stem or progenitor cell of any of the preceding embodiments, which does not comprise a mutation in any predicted off-target site, e.g., in any site listed in FIG. 30, e.g., at any of SEQ ID NOS: 99-112.
14. The genetically engineered hematopoietic stem or progenitor cell of any of the preceding embodiments, which does not comprise a mutation in any predicted off-target site listed in FIG. 29, e.g., at any of SEQ ID NOS: 79-98.
15. The genetically engineered hematopoietic stem or progenitor cell of any of the preceding embodiments, wherein the genetic mutation is a substitution (e.g., a single nucleotide variant), an insertion, or a deletion, or a combination thereof.
16. The genetically engineered hematopoietic stem or progenitor cell of embodiment 15, wherein the deletion is fully within SEQ ID NO: 50, SEQ ID NO: 51, or SEQ ID NO: 58.
17. The genetically engineered hematopoietic stem or progenitor cell of embodiment 15 or 16, wherein the deletion is 1, 2, 4, 5, 7, 8, 10, 11, 13, 14, 16, or 17 nucleotides in length.
18. The genetically engineered hematopoietic stem or progenitor cell of embodiment 15, wherein the deletion extends outside of SEQ ID NO: 50, SEQ ID NO: 51, or SEQ ID NO: 58.
19. The genetically engineered hematopoietic stem or progenitor cell of any of the preceding embodiments, wherein the genetic mutation results in a frameshift.
20. The genetically engineered hematopoietic stem or progenitor cell of any of the preceding embodiments, wherein the genetic mutation results in a reduced expression level of wild-type CD33 as compared with a wild-type counterpart cell (e.g., less than 50%, 40%, 30%, 20%, 15%, 10%, or 5% of the level in the wild-type counterpart cell).
21. The genetically engineered hematopoietic stem or progenitor cell of any of the preceding embodiments, wherein the genetically engineered hematopoietic stem or progenitor cell has a reduced level of wild-type CD33 as compared with a wild-type counterpart cell (e.g., less than 50%, 40%, 30%, 20%, 15%, 10%, or 5% of the level in the wild-type counterpart cell).
22. The genetically engineered hematopoietic stem or progenitor cell of any of the preceding embodiments, wherein the genetic mutation results in a reduced expression level of CD33 as compared with a wild-type counterpart cell (e.g., less than 50%, 40%, 30%, 20%, 15%, 10%, or 5% of the level in the wild-type counterpart cell).
23. The genetically engineered hematopoietic stem or progenitor cell of any of the preceding embodiments, wherein the genetically engineered hematopoietic stem or progenitor cell has a reduced level of CD33 as compared with a wild-type counterpart cell (e.g., less than 50%, 40%, 30%, 20%, 15%, 10%, or 5% of the level in the wild-type counterpart cell).
24. The genetically engineered hematopoietic stem or progenitor cell of any of the preceding embodiments, wherein the genetic mutation results in a lack of expression of CD33.
25. The genetically engineered hematopoietic stem or progenitor cell of any of the preceding embodiments, which expresses less than 20% of the CD33 expressed by the wild-type counterpart.
26. The genetically engineered hematopoietic stem or progenitor cell of any of the preceding embodiments, wherein the reduced expression level of CD33 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
27. The genetically engineered hematopoietic stem or progenitor cell of embodiment 26, wherein the cell differentiated from the hematopoietic stem or progenitor cell is a myeloblast, monoblast, monocyte, macrophage, or natural killer cell.
28. The genetically engineered hematopoietic stem or progenitor cell of any of the preceding embodiments, which does not express CD33.
29. The genetically engineered hematopoietic stem or progenitor cell of any of the preceding embodiments, which is CD34^{+.}
30. The genetically engineered hematopoietic stem or progenitor cell of any of the preceding embodiments, which is from bone marrow cells or peripheral blood mononuclear cells of a subject.
31. The genetically engineered hematopoietic stem or progenitor cell of embodiment 30, wherein the subject is a human patient having a hematopoietic malignancy.
32. The genetically engineered hematopoietic stem or progenitor cell of embodiment 30, wherein the subject is a healthy human donor (e.g., an HLA-matched donor).
33. The genetically engineered hematopoietic stem or progenitor cell of any of the preceding embodiments, which was made by a process comprising contacting the endogenous CD33 gene with a nuclease chosen from a CRISPR endonuclease, a zinc finger nuclease (ZFN), a transcription activator-like effector-based nuclease (TALEN), or a meganuclease (e.g., by contacting the cell with the nuclease or a nucleic acid encoding the nuclease).
34. A cell population, comprising a plurality of the genetically engineered hematopoietic stem or progenitor cells of any of the preceding embodiments (e.g., comprising hematopoietic stem cells, hematopoietic progenitor cells, or a combination thereof).
35. A cell population comprising a plurality of genetically engineered hematopoietic stem or progenitor cells which comprise a genetic mutation in the exon 3 of an endogenous CD33 gene, wherein the genetic mutation is at a site having a sequence of ATCCCTGGCACTCTAGAACC (SEQ ID NO: 50).
36. A cell population comprising a plurality of genetically engineered hematopoietic stem or progenitor cells which comprise a genetic mutation in the exon 3 of an endogenous CD33 gene, wherein the genetic mutation is at a site having a sequence of ATCCCTGGCACTCTAGAACC (SEQ ID NO: 50), wherein the genetic mutation results in a reduced expression level of CD33 as compared with a wild-type counterpart cell population.
37. A cell population comprising a plurality of genetically engineered hematopoietic stem or progenitor cells which comprise a genetic mutation in the exon 3 of an endogenous CD33 gene, wherein the genetic mutation is at a site having a sequence of ATCCCTGGCACTCTAGAACC (SEQ ID NO: 50), wherein the genetic mutation results in a reduced expression level of CD33 that is less than 20% of the level of CD33 in a wild-type counterpart cell population.
38. A cell population comprising a plurality of genetically engineered hematopoietic stem or progenitor cells which comprise a genetic mutation in the exon 3 of an endogenous CD33 gene, wherein the genetic mutation is at a site having a sequence of CCTCACTAGACTTGACCCAC (SEQ ID NO: 58).
39. A cell population comprising a plurality of genetically engineered hematopoietic stem or progenitor cells which comprise a genetic mutation in the exon 3 of an endogenous CD33 gene, wherein the genetic mutation is at a site having a sequence of CCTCACTAGACTTGACCCAC (SEQ ID NO: 58), wherein the genetic mutation results in a reduced expression level of CD33 as compared with a wild-type counterpart cell population.
40. A cell population comprising a plurality of genetically engineered hematopoietic stem or progenitor cells which comprise a genetic mutation in the exon 3 of an endogenous CD33 gene, wherein the genetic mutation is at a site having a sequence of CCTCACTAGACTTGACCCAC (SEQ ID NO: 58), wherein the genetic mutation results in a reduced expression level of CD33 that is less than 20% of the level of CD33 in a wild-type counterpart cell population.
41. The cell population of any of embodiments 34-40, which further comprises one or more cells that comprise one or more non-engineered CD33 genes.
42. The cell population of embodiments 34-41, which further comprises one or more cells that are homozygous wild-type for CD33.
43. The cell population of any of embodiments 34-42 which further comprises one or more cells that are heterozygous wild-type for CD33.
44. The cell population of any of embodiments 34-43 wherein at least 50%, 60%, 70%, 80%, 85%, 90%, or 95% of cells in the population comprise genetic mutations in two copies of CD33.
45. The cell population of any of embodiments 34-44, which expresses less than 20% of the CD33 expressed by a wild-type counterpart cell population.
46. The cell population of any of embodiments 34-45, wherein the reduced expression level of CD33 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
47. The cell population of embodiment 46, wherein the cell differentiated from the hematopoietic stem or progenitor cell is a myeloblast, monoblast, monocyte, macrophage, or natural killer cell.
48. The cell population of any of embodiments 34-47, which comprises hematopoietic stem cells and hematopoietic progenitor cells.
49. A pharmaceutical composition comprising the genetically engineered hematopoietic stem or progenitor cell of any of embodiments 1-33.
50. A pharmaceutical composition comprising the cell population of any of embodiments 34-48.
51. A method of making the genetically engineered hematopoietic stem or progenitor cell of any of embodiments 1-33, or the cell population of any of embodiments 34-48, which comprises:

(i) providing a hematopoietic stem or progenitor cell (e.g., a wild-type hematopoietic stem or progenitor cell), and

(ii) introducing into the cell a nuclease (e.g., an endonuclease) that cleaves the site, thereby producing a genetically engineered hematopoietic stem or progenitor cell.
52. The method of embodiment 51, wherein (ii) comprises introducing into the cell a gRNA that binds the site and an endonuclease that binds the gRNA.
53. The method of embodiment 51, wherein the endonuclease is a ZFN, TALEN, or meganuclease.
54. A guide ribonucleic acid (gRNA) comprising a sequence of any of:

(SEQ ID NO: 67)
AUCCCUGGCACUCUAGAACC,
(SEQ ID NO: 68)
GGCCGGGUUCUAGAGUGCCA,
or
(SEQ ID NO: 70)
CCUCACUAGACUUGACCCAC,

or the reverse complement thereof, or a sequence having at least 90% or 95% identity to any of the foregoing, or a sequence having no more than 1, 2, or 3 mutations relative to any of the foregoing.
55. A guide ribonucleic acid (gRNA) comprising a spacer sequence that is at least 90% identical to SEQ ID NO:70.
56. The gRNA of embodiment 55, wherein the spacer sequence comprises SEQ ID NO:70.
57. A guide ribonucleic acid (gRNA) comprising a spacer sequence that is at least 90% identical to SEQ ID NO:67.
58. The gRNA of embodiment 57, wherein the spacer sequence comprises SEQ ID NO:67.
59. A guide ribonucleic acid (gRNA) comprising at least 18 (e.g., 19, 20, 21, or 22) sequential nucleotides from the sequence of any of:

(SEQ ID NO: 71)
CCUCAUCCCUGGCACUCUAGAACCCGGC,
(SEQ ID NO: 72)
GAGUGGCCGGGUUCUAGAGUGCCAGGGA,
or
(SEQ ID NO: 73)
UUCUCCUCACUAGACUUGACCCACAGGC,

or the reverse complement thereof, or a sequence having at least 90% or 95% identity to any of the foregoing, or a sequence having no more than 1, 2, or 3 mutations relative to any of the foregoing.
60. The gRNA of embodiment 59, wherein the at least 18, 19, 20, 21, or 22 sequential nucleotides start at position 1 of said sequence.
61. The gRNA of embodiment 59, wherein the at least 18, 19, 20, 21, or 22 sequential nucleotides start at position 2 of said sequence.
62. The gRNA of embodiment 59, wherein the at least 18, 19, 20, 21, or 22 sequential nucleotides start at position 3 of said sequence.
63. The gRNA of embodiment 59, wherein the at least 18, 19, 20, 21, or 22 sequential nucleotides start at position 4 of said sequence.
64. The gRNA of embodiment 59, wherein the at least 18, 19, 20, 21, or 22 sequential nucleotides start at position 5 of said sequence.
65. The gRNA of embodiment 59, wherein the at least 18, 19, 20, 21, or 22 sequential nucleotides start at position 6 of said sequence.
66. The gRNA of embodiment 59, wherein the at least 18, 19, 20, 21, or 22 sequential nucleotides start at position 7 of said sequence.
67. The gRNA of embodiment 59, wherein the at least 18, 19, 20, or 21 sequential nucleotides start at position 8 of said sequence.
68. The gRNA of embodiment 59, wherein the at least 18, 19, or 20 sequential nucleotides start at position 9 of said sequence.
69. The gRNA of any of embodiments 54 or 59-68, wherein the 2 mutations are not adjacent to each other.
70. The gRNA of any of embodiments 54 or 59-68, wherein none of the 3 mutations are adjacent to each other.
71. The gRNA of any of embodiments 54 or 59-70, wherein the 1, 2, or 3 mutations are substitutions.
72. The gRNA of any of embodiments 54 or 59-70, wherein one or more of the mutations is an insertion or deletion.
73. The gRNA of any one of embodiments 54-72, wherein the spacer sequence is about 18-23, e.g., 20 nucleotides in length.
74. The gRNA of any one of embodiments 54-73, wherein the spacer sequence has a GC content of 45%-65% or 50-60%, e.g., 55%.
75. The gRNA of any of embodiments 54-74, which comprises one or more chemical modifications (e.g., a chemical modification to a nucleobase, sugar, or backbone portion).
76. The gRNA of any of embodiments 54-75, which comprises one or more 2′O-methyl nucleotide, e.g., at a position described herein.
77. The gRNA of any of embodiments 54-76, which comprises one or more phosphorothioate or thioPACE linkage, e.g., at a position described herein.
78. The gRNA of any of embodiments 54-77, which is a single-molecule gRNA (sgRNA).
79. The gRNA of any of embodiments 54-78, which binds Cas9.
80. The gRNA of any of embodiments 54-79, which binds to a tracrRNA.
81. The gRNA of any of embodiments 54-79, which comprises a scaffold sequence.
82. A kit or composition comprising:

a) a gRNA of any of embodiments 54-81, or a nucleic acid encoding the gRNA, and

b) a second gRNA, or a nucleic acid encoding the second gRNA.
83. The kit or composition of embodiment 82, wherein the gRNA of (a) comprises a spacer sequence of CCUCACUAGACUUGACCCAC (SEQ ID NO:70).
84. The kit or composition of embodiment 82, wherein the gRNA of (a) comprises a spacer sequence of AUCCCUGGCACUCUAGAACC (SEQ ID NO:67).
85. The kit or composition of any of embodiments 82-84, wherein the second gRNA targets a lineage-specific cell-surface antigen.
86. The kit or composition of any of embodiments 82-85 wherein the second gRNA targets a lineage-specific cell-surface antigen other than CD33.
87. The kit or composition of any of embodiments 82-86, wherein the second gRNA targets CLL-1.
88. The kit or composition of any of embodiments 82-87, wherein the second gRNA comprises a spacer sequence of GUUGUAGAGAAAUAUUUCUC (SEQ ID NO: 115), GGAGAGGUUCCUGAUCUUGU (SEQ ID NO: 116), or UGAAUAUCUCCAACAAGAUC (SEQ ID NO: 119).
89. The kit or composition of any of embodiments 82-88, wherein the gRNA of (a) comprises a spacer sequence according to SEQ ID NO: 70 and the second gRNA comprises a spacer sequence according to SEQ ID NO: 115.
90. The kit or composition of any of embodiments 82-88, wherein the gRNA of (a) comprises a spacer sequence according to SEQ ID NO: 70 and the second gRNA comprises a spacer sequence according to SEQ ID NO: 116.
91. The kit or composition of any of embodiments 82-88, wherein the gRNA of (a) comprises a spacer sequence according to SEQ ID NO: 67 and the second gRNA comprises a spacer sequence according to SEQ ID NO: 115.
92. The kit or composition of any of embodiments 82-88, wherein the gRNA of (a) comprises a spacer sequence according to SEQ ID NO: 67 and the second gRNA comprises a spacer sequence according to SEQ ID NO: 116.
93. The kit or composition of any of embodiments 82-88, wherein the gRNA of (a) comprises a spacer sequence according to SEQ ID NO: 67 and the second gRNA comprises a spacer sequence according to SEQ ID NO: 70.
94. The kit or composition of any of embodiments 82-88, wherein the gRNA of (a) comprises a spacer sequence according to SEQ ID NO: 115 and the second gRNA comprises a spacer sequence according to SEQ ID NO: 116.
95. The kit or composition of any of embodiments 82-94, which further comprises a third gRNA, or a nucleic acid encoding the third gRNA.
96. The kit or composition of embodiment 95, wherein the third gRNA targets a lineage-specific cell-surface antigen.
97. The kit or composition of embodiment 95, wherein the third gRNA targets CD33 or CLL-1.
98. The kit or composition of any of embodiments 95-97, wherein the gRNA of (a) comprises a spacer sequence according to SEQ ID NO: 70, the second gRNA comprises a spacer sequence according to SEQ ID NO: 115, and the third gRNA comprises a spacer sequence of SEQ ID NO: 116.
99. The kit or composition of any of embodiments 95-97, wherein the gRNA of (a) comprises a spacer sequence according to SEQ ID NO: 67, the second gRNA comprises a spacer sequence according to SEQ ID NO: 115, and the third gRNA comprises a spacer sequence of SEQ ID NO: 116.
100. The kit or composition of any of embodiments 95-97, wherein the gRNA of (a) comprises a spacer sequence according to SEQ ID NO: 67, the second gRNA comprises a spacer sequence according to SEQ ID NO: 70, and the third gRNA comprises a spacer sequence of SEQ ID NO: 116.
101. The kit or composition of any of embodiments 95-97, wherein the gRNA of (a) comprises a spacer sequence according to SEQ ID NO: 67, the second gRNA comprises a spacer sequence according to SEQ ID NO: 70, and the third gRNA comprises a spacer sequence of SEQ ID NO: 115.
102. The kit or composition of any of embodiments 95-101, which further comprises a fourth gRNA, or a nucleic acid encoding the fourth gRNA.
103. The kit or composition of embodiment 102, wherein the fourth gRNA targets a lineage-specific cell-surface antigen.

104. The kit or composition of embodiment 102, wherein the fourth gRNA targets CD33 or CLL-1 .
105. The kit or composition of embodiment 102, wherein the gRNA of (a) comprises a spacer sequence according to SEQ ID NO: 67, the second gRNA comprises a spacer sequence according to SEQ ID NO: 70, the third gRNA comprises a spacer sequence of SEQ ID NO: 115, and the fourth gRNA comprises a spacer sequence of SEQ ID NO: 116.
106. The kit or composition of any of embodiments 102-105, wherein the gRNA of (a), the second gRNA, the third gRNA, and the fourth gRNA are admixed.
107. The kit or composition of any of embodiments 102-105, wherein the gRNA of (a), the second gRNA, the third gRNA, and the fourth gRNA are in separate containers.
108. The kit or composition of any of embodiments 82-105, wherein (a) and (b) are admixed.
109. The kit or composition of any of embodiments 82-105, wherein (a) and (b) are in separate containers.
110. The kit or composition of any of embodiments 82-109, wherein the nucleic acid of (a) and the nucleic acid of (b) are part of the same nucleic acid.
111. The kit or composition of any of embodiments 82-109, wherein the nucleic acid of (a) and the nucleic acid of (b) are separate nucleic acids.
112. A genetically engineered hematopoietic cell (e.g., hematopoietic stem or progenitor cell), which comprises one or more of (e.g., 2, 3, or all of):

(a) a genetic mutation in the exon 3 of an endogenous CD33 gene, wherein the genetic mutation is at a site having a sequence of ATCCCTGGCACTCTAGAACC (SEQ ID NO: 50);

(b) a genetic mutation in the exon 3 of an endogenous CD33 gene, wherein the genetic mutation is at a site having a sequence of CCTCACTAGACTTGACCCAC (SEQ ID NO: 58).

(c) a genetic mutation in CLL-1 at a site having a sequence of

(SEQ ID NO: 117)
GTTGTAGAGAAATATTTCTC

(d) a genetic mutation in CLL-1 at a site having a sequence of

(SEQ ID NO: 118)
GGAGAGGTTCCTGATCTTGT.

113. The hematopoietic cell of embodiment 112, which comprises (a) and (b).
114. The hematopoietic cell of embodiment 112, which comprises (a) and (c).
115. The hematopoietic cell of embodiment 112, which comprises (a) and (d).
116. The hematopoietic cell of embodiment 112, which comprises (b) and (c).
117. The hematopoietic cell of embodiment 112, which comprises (b) and (d).
118. The hematopoietic cell of embodiment 112, which comprises (c) and (d).
119. The hematopoietic cell of embodiment 112, which comprises (a), (b) and (c).
120. The hematopoietic cell of embodiment 112, which comprises (a), (b) and (d).
121. The hematopoietic cell of embodiment 112, which comprises (a), (c) and (d).
122. The hematopoietic cell of embodiment 112, which comprises (b), (c) and (d).
123. The hematopoietic cell of embodiment 112, which comprises (a), (b), (c) and (d).
124. The hematopoietic cell of any of embodiments 112-123, which comprises a deletion spanning from a position within SEQ ID NO: 50 to a position within SEQ ID NO: 58.
125. The hematopoietic cell of any of embodiments 112-124, which comprises a deletion spanning from a position within SEQ ID NO: 117 to a position within SEQ ID NO: 118.
126. Use of a gRNA of any of embodiments 54-81 or a composition or kit of any of embodiments 82-111 for reducing expression of CD33 in a sample of hematopoietic cells stem or progenitor cells using a CRISPR/Cas9 system.
127. Use of a CRISPR/Cas9 system for reducing expression of CD33 in a sample of hematopoietic cells stem or progenitor cells, wherein the gRNA of the CRISPR/Cas9 system is a gRNA of any of embodiments 54-81 or gRNAs of a composition or kit of any of embodiments 82-111.
128. A method of producing a genetically engineered hematopoietic stem or progenitor cell, comprising:

(i) providing a hematopoietic stem or progenitor cell (e.g., a wild-type hematopoietic stem or progenitor cell), and

(ii) introducing into the cell (a) a guide RNA (gRNA) of any of embodiments 22-39 or gRNAs of a composition or kit of any of embodiments 82-111; and (b) a nuclease (e.g., an endonuclease) that binds the gRNA (e.g., a Cas9 endonuclease),

thereby producing a genetically engineered hematopoietic stem or progenitor cell.
129. A method of producing a genetically engineered hematopoietic stem or progenitor cell, comprising:

(i) providing a genetically engineered hematopoietic stem and/or progenitor cell, and

(ii) introducing into the cell (a) a guide RNA (gRNA) that comprises a nucleotide sequence at least 90% identical to SEQ ID NO: 67 or SEQ ID NO: 70; and (b) a Cas9 endonuclease, thereby producing a genetically engineered hematopoietic stem and/or progenitor cell having a reduced expression level of CD33.
130. A method of producing a genetically engineered hematopoietic stem or progenitor cell, comprising:

(i) providing a hematopoietic stem or progenitor cell, and

(ii) introducing into the cell (a) a guide RNA (gRNA) that comprises a nucleotide sequence at least 90% identical to SEQ ID NO: 67 or SEQ ID NO: 70; and (b) a Cas9 endonuclease, thereby producing the genetically engineered hematopoietic stem or progenitor cell.
131. A method of producing a genetically engineered hematopoietic stem or progenitor cell having a reduced expression level of CD33, comprising:

(i) providing a hematopoietic stem or progenitor cell, and

(ii) introducing into the cell (a) a guide RNA (gRNA) that comprises a nucleotide sequence at least 90% identical to SEQ ID NO: 67 or SEQ ID NO: 70; and (b) a Cas9 endonuclease, thereby producing a genetically engineered hematopoietic stem or progenitor cell having a reduced expression level of CD33.
132. A method of producing a genetically engineered hematopoietic stem or progenitor cell, comprising:

(i) providing a hematopoietic stem or progenitor cell, and

(ii) introducing into the cell (a) a guide RNA (gRNA) that comprises a nucleotide sequence according to SEQ ID NO: 67; and (b) a Cas9 endonuclease, thereby producing the genetically engineered hematopoietic stem or progenitor cell.
133. A method of producing a genetically engineered hematopoietic stem or progenitor cell, comprising:

(i) providing a hematopoietic stem or progenitor cell, and

(ii) introducing into the cell (a) a guide RNA (gRNA) that comprises a nucleotide sequence according to SEQ ID NO: 70; and (b) a Cas9 endonuclease, thereby producing the genetically engineered hematopoietic stem or progenitor cell.
134. The method or use of any of embodiments 51-53 or 126-133, which results in the genetically engineered hematopoietic stem or progenitor cell having a reduced expression level of CD33 as compared with a wild-type counterpart cell.
135. The method or use of any of embodiments 51-53 or 126-134, which results in the genetically engineered hematopoietic stem or progenitor cell having a reduced expression level of CD33 that is less than 20% of the level of CD33 in a wild-type counterpart cell.
136. The method or use of any of embodiments 51-53 or 126-135, which is performed on a plurality of hematopoietic stem or progenitor cells.
137. The method or use of any of embodiments 51-53 or 126-136, which is performed on a cell population comprising a plurality of hematopoietic stem cells and a plurality of hematopoietic progenitor cells.
138. The method or use of any of embodiments 51-53 or 126-137, which produces a cell population according to any of embodiments 34-48.
139. The method of any of embodiments 128-138, wherein the nucleic acids of (a) and (b) are encoded on one vector, which is introduced into the cell.
140. The method of embodiment 139, wherein the vector is a viral vector.
141. The method of embodiment 139, wherein (a) and (b) are introduced into the cell as a pre-formed ribonucleoprotein complex.
142. The method of embodiment 141, wherein the ribonucleoprotein complex is introduced into the cell via electroporation.
143. The method of any of embodiments 128-142, wherein the endonuclease (e.g., Cas9 endonuclease) is introduced into the cell by delivering into the cell a nucleic acid molecule (e.g., an mRNA molecule) encoding the Cas9 endonuclease.
144. The method of any of embodiments 128-143, wherein the gRNA is a single-molecule guide RNA (sgRNA).
145. The method of any of embodiments 128-144, wherein the gRNA is a modified sgRNA.
146. The method of any of embodiments 128-145, wherein the gRNA is a chemically modified sgRNA.
147. The method of any of embodiments 128-146, wherein the hematopoietic stem or progenitor cell is CD34^{+.}
148. The method of any of embodiments 128-147, wherein the hematopoietic stem or progenitor cell is from bone marrow cells or peripheral blood mononuclear cells (PBMCs) of a subject.
149. The method of embodiment 148, wherein the subject has a hematopoietic disorder.
150. The method of embodiment 148, wherein the subject is a healthy donor.
151. The method or use of any of embodiments 51-53 or 126-150, which results in a mutation that causes a reduced expression level of CD33 as compared with a wild-type counterpart cell.
152. The method or use of any of embodiments 51-53 or 126-151, which results in a mutation that causes a reduced expression level of wild-type CD33 as compared with a wild-type counterpart cell.
153. The method or use of any of embodiments 51-53 or 126-152, which produces a genetically engineered hematopoietic stem or progenitor cell having a reduced expression level of CD33 as compared with a wild-type counterpart cell.
154. The method or use of any of embodiments 51-53 or 126-153, which produces a genetically engineered hematopoietic stem or progenitor cell having a reduced expression level of wild-type CD33 as compared with a wild-type counterpart cell.
155. A genetically engineered hematopoietic stem or progenitor cell, which is produced by a method or use of any of embodiments 51-53 or 126-154.
156. A method of treating a hematopoietic disorder, comprising administering to a subject in need thereof an effective amount of the genetically engineered hematopoietic stem or progenitor cell of any of embodiments 1-33, or the cell population of any of embodiments 34-48, or a hematopoietic cell of any of embodiments 112-125.
157. A genetically engineered hematopoietic stem or progenitor cell of any of embodiments 1-33, a cell population of any of embodiments 34-38, or a hematopoietic cell of any of embodiments 112-125, for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33.
158. An agent that targets CD33, wherein the agent comprises an antigen-binding fragment that binds CD33, for use in treating a hematopoietic disorder, wherein the treating comprises administering to a patient in need thereof an effective amount of the agent that targets CD33, and further comprises administering to the patient an effective amount of a genetically engineered hematopoietic stem or progenitor cell of any of embodiments 1-33, a cell population of any of embodiments 34-38, or a hematopoietic cell of any of embodiments 112-125.
159. A combination of a genetically engineered hematopoietic stem or progenitor cell of any of embodiments 1-33, a cell population of any of embodiments 34-38, or a hematopoietic cell of any of embodiments 112-125, and an agent that targets CD33, wherein the agent comprises an antigen-binding fragment that binds CD33, for use in treating a hematopoietic disorder, wherein the treating comprises administering to a patient in need thereof an effective amount of the genetically engineered hematopoietic stem or progenitor cell or the cell population, and the agent that binds CD33.
160. A genetically engineered hematopoietic stem or progenitor cell of any of embodiments 1-33, a cell population of any of embodiments 34-38, or a hematopoietic cell of any of embodiments 112-125, for use in cancer immunotherapy.
161. A genetically engineered hematopoietic stem or progenitor cell of any of embodiments 1-33, a cell population of any of embodiments 34-38, or a hematopoietic cell of any of embodiments 112-125 for use in cancer immunotherapy, wherein the patient has a hematopoietic disorder.
162. A genetically engineered hematopoietic stem or progenitor cell of any of embodiments 1-33, a cell population of any of embodiments 34-38, or a hematopoietic cell of any of embodiments 112-125 for use in hematopoietic repopulation of a patient having a hematopoietic disorder.
163. A genetically engineered hematopoietic stem or progenitor cell of any of embodiments 1-33, a cell population of any of embodiments 34-38, or a hematopoietic cell of any of embodiments 112-125 for use in a method of treating a hematopoietic disorder, whereby the genetically engineered hematopoietic stem or progenitor cell described herein or a cell population described herein repopulate the patient.
164. A genetically engineered hematopoietic stem or progenitor cell of any of embodiments 1-33, a cell population of any of embodiments 34-38, or a hematopoietic cell of any of embodiments 112-125 for use in reducing cytotoxic effects of an agent that targets CD33 in immunotherapy.
165. A genetically engineered hematopoietic stem or progenitor cell of any of embodiments 1-33, a cell population of any of embodiments 34-38, or a hematopoietic cell of any of embodiments 112-125 for use in an immunotherapy method using an agent that targets CD33, whereby the genetically engineered hematopoietic stem or progenitor cell described herein or a cell population described herein reduces cytotoxic effects of the agent that targets CD33.
166. The method, cell, agent, or combination of any of embodiments 156-165, wherein the genetically engineered hematopoietic stem or progenitor cell or the cell population is administered concomitantly with the agent that targets CD33.
167. The method, cell, agent, or combination of any of embodiments 156-165, wherein the genetically engineered hematopoietic stem or progenitor cell or the cell population is administered prior to the agent that targets CD33.
168. The method, cell, agent, or combination of any of embodiments 156-165, wherein the agent that targets CD33 is administered prior to the genetically engineered hematopoietic stem or progenitor cell or the cell population.
169. The method, cell, agent, use, or combination of any of embodiments 156-168, wherein the hematopoietic disorder is a hematopoietic malignancy.
170. The method, cell, agent, use, or combination of any of embodiments 156-169, further comprising administering to the subject an effective amount of an agent that targets CD33, and wherein the agent comprises an antigen-binding fragment that binds CD33.
171. The method, cell, agent, use, or combination of embodiment 170, wherein the agent that targets CD33 is an immune cell expressing a chimeric antigen receptor (CAR), which comprises the antigen-binding fragment that binds CD33.
172. The method, cell, agent, use, or combination of embodiment 171, wherein the immune cell is a T cell.
173. The method, cell, agent, use, or combination of any of embodiments 170-172, wherein the immune cells, the genetically engineered hematopoietic stem and/or progenitor cell, or both, are allogeneic.
174. The method, cell, agent, use, or combination of embodiment 170-173, wherein the immune cells, the genetically engineered hematopoietic stem and/or progenitor cell, or both, are autologous.
175. The method, cell, agent, use, or combination of any of embodiments 170-174, wherein the antigen-binding fragment in the chimeric receptor is a single-chain antibody fragment (scFv) that specifically binds human CD33.
176. The method, cell, agent, use, or combination of any of embodiments 170-175, wherein the subject is a human patient having Hodgkin's lymphoma, non-Hodgkin's lymphoma, leukemia, or multiple myeloma.
177. The method, cell, agent, use, or combination of embodiment 176, wherein the subject is a human patient having leukemia, which is acute myeloid leukemia, chronic myelogenous leukemia, acute lymphoblastic leukemia, or chronic lymphoblastic leukemia.

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 presents an exemplary illustration of type 0, type 1, type 2, and type 3 lineage-specific antigens.

FIG. 2 is a schematic showing an immune cell expressing a chimeric receptor that targets the type 0 lineage-specific cell-surface antigen, CD307. Multiple myeloma (MM) cells expressing CD307 as well other cells expressing CD307, such as plasma cells, are targeted by the immune cells expressing the anti-CD307chimeric receptor.

FIG. 3 is a schematic showing an immune cell expressing a chimeric receptor that targets the type 2 lineage-specific cell-surface antigen, CD33. Acute myeloid leukemia (AML) cells expressing CD33. Human hematopoietic stem cells (HSC) are genetically engineered to be deficient in CD33 and therefore not recognized by the immune cells expressing the anti-CD33 chimeric receptor. The HSC are able to give rise to myeloid cells.

FIG. 4 is a schematic showing genome editing using a CRISPR/Cas system. A sgRNA hybridizes to a portion of an exon of a lineage-specific cell-surface antigen, and the Cas9 endonuclease cleaves upstream of the Protospacer Adjacent Motif (PAM) Sequence (5′-NGG-3′). The sequences, from top to bottom, correspond to SEQ ID NOs: 45 and 46.

FIG. 5 is a schematic showing a genome editing strategy using the CRISPR/Cas9 system to disrupt CD33. A PX458 vector encoding a Cas9 protein and a guide RNA targeting CD33 was nucleofected into K-562 cells, a human leukemic cell line. Flow cytometry was performed on the cell population using an anti-CD33 antibody prior to (top plot) and after (bottom plot) delivery of Cas9 and guide RNA to the cells. The genome editing resulted in the deletion of a coding region of the gene and a significant reduction in CD33 from the cell surface.

FIG. 6 is a schematic showing a genome editing strategy using the CRISPR/Cas9 system to disrupt CD45RA. A PX458 vector encoding a Cas9 protein and a guide RNA targeting CD45RA was nucleofected into TIB-67 reticulum cell sarcoma mouse macrophage-like cells. Flow cytometry was performed on the cell population using an anti-CD45RA antibody prior to (top plot) and after (bottom plot) delivery of Cas9 and guide RNA to the cells. The genome editing resulted in the deletion of a coding region of the gene and a significant reduction in CD45RA from the cell surface.

FIGS. 7A-7D show schematics of example chimeric receptors comprising antigen-binding fragments that target CD33. FIG. 7A: a generic chimeric receptor targeting CD33 comprising an anti-CD33 scFv, hinge domain, transmembrane domain, co-stimulatory domain, and signaling domain. FIG. 7B: a chimeric receptor targeting CD33 comprising an anti-CD33 scFv, hinge domain from CD8, transmembrane domain from CD8, and intracellular domains from CD28 and CD3ζ. FIG. 7C: a chimeric receptor targeting CD33 comprising an anti-CD33 scFv, hinge domain from CD8, transmembrane domain from CD8, and intracellular domains from ICOS (or CD27, 4-1BB, or OX-40) and CD3ζ. FIG. 7D: a chimeric receptor targeting CD33 comprising an anti-CD33 scFv, hinge domain from CD8, transmembrane domain from CD8, and intracellular domains from OX40, CD28, and CD3ζ.

FIG. 8 is a schematic of an immunotoxin.

FIGS. 9A-9B show expression of anti-CD33 chimeric receptors expressed in K562 cells transduced with an empty vector or vector encoding an anti-CD33 chimeric receptor. FIG. 9A: Western blot using a primary antibody that recognizes CD3ζ. The table provides the estimated molecular weight of each of the chimeric receptors tested. FIG. 9B: Flow cytometric analysis showing an increase in the population of cells that stain positive for the anti-CD33 chimeric receptor.

FIGS. 10A-10C show the anti-CD33 chimeric receptors bind to CD33. FIG. 10A: Ponceau stained protein gel. Lanes 1,3,5 : CD33 molecule. Lanes 2,4,6: CD33 mol +APC Conjugate. FIG. 10B: Western blot using a primary antibody that recognizes CD3ζ. Lanes 1, 3, and 5 contain the chimeric receptors co-incubated with CD33 molecules, and lanes 2, 4, and 6 contain the chimeric receptors co-incubated with a CD33-APC conjugate. FIG. 10C: Flow cytometric analysis showing an increase in the population of cells that express anti-CD33 chimeric receptors and bind CD33.

FIGS. 11A-11B show cytotoxicity of K562 cells by NK92 cells expressing the indicated chimeric receptors. FIG. 11A: CART1 and CART2 compared to empty HIVzsG vector. FIG. 11B: CART3 compared to empty HIVzsG vector.

FIGS. 12A-12B show cytotoxicity (expressed as percent cytotoxicity on the y-axis) of K562 cells deficient in CD33 by NK92 cells expressing the indicated chimeric receptors. FIG. 12A: unsorted population of K562 cells pretreated with CD33-targeting CRISPR/Cas reagents. FIG. 12B: single clones of K562 cells deficient in CD33. The columns, from left to right, correspond to empty HIVzsG vector, CART1, CART2, and CART3.

FIGS. 13A-13B show flow cytometric analysis of primary T cell populations. FIG. 13A: sorting of cells based on expression of T cell markers C4⁺, CD8⁺, or both CD4⁺CD8⁺. FIG. 13B: relative expression of CD33 on the indicated populations of primary T cells.

FIGS. 14A-14B show cytotoxicity of K562 cells by primary T cells expressing the indicated chimeric receptors. FIG. 14A: CD4⁺ T cells. FIG. 14B: CD4⁺/CD8⁺ (CD 4/8) and CD8⁺ (CD8).

FIG. 15 shows flow cytometric analysis of CD33 editing in K562 cells using the CRISPR/Cas9 system and two different gRNAs (Crispr3, right top panel, and Crispr5, right bottom panel).

FIGS. 16A-16C show K562 cells deficient in CD33 present normal cell proliferation and erythropoietic differentiation. FIG. 16A: flow cytometric analysis of the indicated cell populations at day 1+50 μM hemin. FIG. 16B: flow cytometric analysis of the indicated cell populations at day 9. FIG. 16C: MTT cell proliferation assay.

FIGS. 17A-17C show flow cytometric analysis of CD33 editing in human CD34⁺ cells using the CRISPR/Cas9 system and two different gRNAs (crispr3, bottom left panel, and crispr5, bottom right panel). FIG. 17A: flow cytometric analysis of CD33 editing in human CD34⁺ cells using the CRISPR/Cas9 system. FIG. 17B: crispr3. FIG. 17C: crispr5.

FIG. 18 shows colony formation for human CD34⁺/CD33⁻ cells as compared to human CD34⁺/CD33⁺ cells. The columns, from left to right, correspond to no lentivirus infection, empty vector control, crisp1, crispr 3, and crispr5.

FIGS. 19A-19F show CRISPR/Cas9-mediated genetic ablation of the CD33 antigen. FIG. 19A shows the approach: stem cells, either mobilized or cord blood obtained from a donor are genetically manipulated to ablate CD33 expression using gene editing technology such as CRISPR/Cas9 and transplanted to relapsed patients eligible for HSCT. Subsequent to transplantation, T-cells, from an allogeneic donor will be genetically manipulated, using viral delivery system, to express chimeric antigen receptors targeting CD33 and infused in the recipient. Alternatively, patients can receive ADC (GO) either alone or in combination with CAR-T. FIGS. 19B-19F show CD33 expression and its ablation in human cells. FIG. 19B: Expression of CD33 in human AML cell line HL-60, in human primary CD34⁺CD33^{WT} cells from bone marrow (BM) and cord blood (CB) and human primary CD34⁺CD33^{Del} after CRISPR/Cas9 mediated ablation. FIG. 19C: Schematic representation of CD33 genomic locus showing exon 2-4 and location and sequence of sgRNA (in bold) targeting CD33. The sequences, from top to bottom, correspond to SEQ ID NOs: 52-53. FIG. 19D: Surface expression of CD33 by flow cytometry after electroporation in CD34⁺CD33^{WT} cells and CD34⁺CD33^{Del}. All cells maintain their stem cells phenotype as assessed by CD90 expression. FIG. 19E: Chromatogram of Sanger sequencing showing a region surrounding the DNA double strand break site, top: CD34⁺CD33^{WT} cells and bottom: CD34⁺CD33^{Del}. The sequences, from top to bottom, correspond to SEQ ID NOs: 54-55. FIG. 19F: 5 to 7 days after electroporation, CD34⁺ cultured cells show consistent deletion of CD33 compared to control.

FIGS. 20A-20E show that the deletion of CD33 does not impair engraftment and hematopoietic repopulation in NSG-SGM3 mice. FIG. 20A: Schematic of experimental design. FIGS. 20B-20C show bone marrow derived CD34⁺ cells engraftment and repopulation: peripheral blood (7 weeks; FIG. 20B) and whole bone marrow (21 weeks; FIG. 20C) post-transplant were analyzed for cells of various lineages, as indicated. CD34⁺CD33^{Del} cells show same engraftment (CD45⁺) as control cells as well as comparable percentage of mature myeloid and lymphoid cells. Bone Marrow CD34⁺CD33^{Del} cells show comparable percentage of myeloid (progenitor CD123⁺, mature CD14⁺) and lymphoid (progenitor CD10⁺, mature CD19⁺), T cells (CD3⁺) and stem cells CD34⁺38⁻. FIGS. 20D-20E show cord blood derived CD34⁺ cells engraftment and repopulation: peripheral blood (9 weeks; FIG. 20D) and bone marrow (21 weeks; FIG. 20E) post-transplant analyzed for cells of various lineages, as indicated. CD34⁺CD33^{Del} cells show same engraftment (CD45⁺) as control cells as well as comparable percentage of mature myeloid and lymphoid cells. Bone Marrow CD34⁺CD33^{Del} cells show comparable percentage of myeloid (progenitor CD123⁺, mature CD14⁺) and lymphoid (progenitor CD10⁺, mature CD19⁺), T cells (CD3⁺) and stem cells CD34⁺38⁻. Data were analyzed using unpaired t test and no significant differences were found in all the groups examined (p>0.05). All data are represented as mean±SEM.

FIGS. 21A-21D show integrated genomic viewer (IGV) screenshots of the genomic region of CD33 (FIG. 21A) and SIGLEC9 (FIG. 21B) genes surrounding the guides in Cas9+sgRNA (top) and Cas9 only (bottom) cells as indicated in the left. The grey bars in the coverage track (indicated on right) show the depth of the reads displayed at each locus. Generally, the coverage should be uniform and hence the bar height should be same but deletions results in dip in the height. The reads track shows all the reads (grey boxes) mapped in this region. The deletions are represented by a solid black line and insertions with diagonally hatched boxes. Reads with thick border are those without a mapped mate. One read in each group was without a mapped mate. Mismatch bases are filled with diamonds, open circles, closed circles, and white for nucleotides A, C, G, and T, respectively. The SIGLEC9 genomic region was selected as a representative region to show absence of indels at off-target site because 1) it belongs to SIGLEC family with homology to CD33, and 2) it has the highest homology within 10 bp of the expected cut site compared to any other guide. Chromosome coordinates at the bottom are based on hg38. FIG. 21C: Scatter plot showing correlation between log 10 mean normalized counts, normalized using DEseq2 method, between CD33 edited cells and control cells. FIG. 21D: Volcano plot showing log2 fold change and −log 10 p-value for genes analyzed using edgeR method, genes that were significantly differentially expressed (p<0.05) are shown as red open circles and CD33 gene is indicated by a left arrow.

FIGS. 22A-22F show CD33 deletion protects CD34⁺ cells from CART33 cytotoxicity in vitro. FIG. 22A: Schematic of CART33 construct. FIG. 22B: Contour plot showing CAR expression in human primary T cells after lentiviral transduction with control (black) or CART33 (green or blue) virus. Percentage transduction in each group is specified next to the plots. CD4+ and CD8+ cells were transduced independently and comixed 1:1 prior to experiment. FIGS. 22C-22F: Cytotoxicity assays. FIG. 22C: CART33 cells or control T were incubated with HL-60 or CD34⁺CD33^{WT} or CD34⁺CD33 ^{Del} and cytotoxicity assessed by flow cytometry. FIGS. 22D-22F: Triple culture cytotoxicity assay. CART33 cells or control T cells were co-incubated with HL-60 and CD34⁺CD33^{WT} (FIG. 22D), or with HL-60 and CD34⁺CD33^{Del} (FIG. 22E), or with CD34⁺CD33^{WT} and CD34⁺CD33^{Del} (FIG. 22F) cells.

FIGS. 23A-23F show the therapy model: CD34⁺CD33^{Del} cells resist CD33-targeted immunotherapy. FIG. 23A: Schematic of experimental design: 5*10⁵ HL-60 and 5*10⁵ CD34⁺CD33^{Del} were injected in NSGM3 mice on day 0. One week after mice were treated with PBS or allogeneic CART33 or control T cells. 3 days after a new group received GO only, while allogeneic CART33 and control T cells injected mice received GO or PBS. Treatment was repeated on week 3. Leukemia progression and CD34⁺CD33^{Del} engraftment were then monitored by serial bone marrow aspiration. FIG. 23B: Monitoring of leukemia burden in bone marrow aspirates. Leukemia cells were gated on Ter119⁻dtomato±. FIG. 23C: Leukemia burden measure via epi-fluorescence quantification of images shown at 3.5 weeks (FIG. 23D) and at 8 weeks (FIG. 23E). Background was removed with untreated mouse (* Imaging control). FIG. 23F: CART33 or GO leukemia clearance does not impair engraftment of CD34⁺CD33^{Del} cells over time (% hCD45⁺ cells), as shown by flow cytometry of bone marrow aspirates. CD34⁺ injected derived human cells were gated on Ter119⁻dtomato⁻, Ly5⁻/H2kd⁻human CD45⁺CART⁻.

FIGS. 24A-24D show that CD34⁺CD33Del HSPC show multilineage engraftment and differentiation in the therapy model. FIG. 24A: CD34⁺CD33^{Del} cells resist CD33-targeted immunotherapy and contribute to myelopoiesis and lymphopoiesis. Left two panel in each condition is monitoring over time of the repopulation of myeloid progenitors and right two panels, lymphoid progenitors and mature cells in BM aspirates. No significant differences were observed between different treatment groups at all timepoints analyzed. FIGS. 24B-24D show that CD34⁺CD33^{WT} cells are sensitive to CD33-targeted immunotherapy. FIG. 24B: A schematic of experimental design: 5*10⁵CD34⁺CD33^{WT} alone or in combination with 5*10⁵HL-60 were injected in NSG-SGM3 mice on day 0. One week after mice were treated with PBS or allogeneic CART33 cells. Leukemia progression and CD34⁺CD33^{WT} engraftment were then monitored by bone marrow aspiration at week 3 for CART33. The same day a group of mice was injected with GO and analyzed 4 days after. FIGS. 24C-24D: BM aspirates show complete elimination of CD33^{WT} leukemia cells (FIG. 24C) and CD33^{WT} primary cells (FIG. 24D) in mice treated with CART33 or GO compared to PBS alone. A significant difference was observed between CART33 and GO compared to PBS. CD34⁺injected derived human cells were gated on Ter119⁻dtomato⁻, Ly5⁻/H2kd⁻human CD45⁺CART⁻. All data are represented as mean±SEM.

FIGS. 25A-25B show additional sgRNA tested to ablate CD33 expression show high level of indels. FIGS. 25A-25B: Schematic representation of CD33 genomic locus showing exon 2-4 and location and sequence of two additional sgRNA targeting CD33 locus. Chromatogram at the bottom are screenshot of Sanger sequencing showing a region surrounding the DNA double strand break site, left: CD34⁺CD33^{WT} cells and right: CD34⁺CD33^{Del}. Guide sequences are highlighted in blue on the chromatogram and appearance of indels is indicated by red downward arrow. The sequences correspond to SEQ ID NOs: 56-57, 75-76, 113, 59 and 77-78, respectively, in order of appearance.

FIGS. 26A-26D show that deletion of CD33 does not impair engraftment and hematopoietic repopulation in NSGM3 mice. FIGS. 26A-26B show bone marrow derived CD34⁺ cells engraftment and repopulation. FIG. 26A: Bone marrow aspirate (15 weeks) post-transplant was analyzed for cells of various lineages, as indicated. CD34⁺CD33 ^{Del} cells show same engraftment (CD45⁺) as control cells as well as comparable percentage of mature myeloid and lymphoid cells. The bar diagram represents summary of individual panels. FIG. 26B: Summary of data from main FIG. 20C. FIGS. 26C-26D show cord blood derived CD34⁺ cells engraftment and repopulation. FIG. 26C: Bone marrow aspirate (16 weeks) post-transplant was analyzed for cells of various lineages, as indicated. CD34⁺CD33 ^{Del} cells show same engraftment (CD45⁺) as control cells as well as comparable percentage of mature myeloid and lymphoid cells. The bar diagram represents summary of individual panels. FIG. 26D: Summary of data from FIG. 20E. No significant differences were observed between both groups in all cell types analyzed (p>0.05), unpaired t test. All data are represented as mean±SEM.

FIG. 27 shows coverage of reads in RNAseq data. Integrated genomic viewer (IGV) screenshot of coverage track showing CD33 genomic regions surrounding the guides in Cas9+sgRNA (n=5; bottom) and Cas9 only (n=5; top) cells as indicated in the left. The grey bars in the coverage track (indicated on right) show the depth of the reads displayed at each locus. Generally, the coverage should be uniform and hence the bar height should be the same, but deletions results in dip in the height of the bars, marked by a dotted rectangle in the lower panel. FIG. 27 discloses SEQ ID NOS 114 and 114, respectively, in order of appearance.

FIG. 28 shows a summary of reads and variants found in whole genome sequencing.

FIG. 29 shows off-target sites for sgRNA 846 (sgRNA: AUCCCUGGCACUCUAGAACC (SEQ ID NO: 67); PAM: CGG). No indels were found within 100 bp of the expected off-target cut site in whole genome sequencing analysis. Mismatch with guide sequence is denoted in bold. FIG. 29 discloses SEQ ID NOS 79-98, respectively, in order of appearance.

FIG. 30 shows off-target sites for sgRNA 811 (sgRNA: CCUCACUAGACUUGACCCAC (SEQ ID NO: 70); PAM: AGG). No indels were found within 100 bp of the expected off-target cut site in whole genome sequencing analysis. Mismatch with guide sequence is denoted in bold. FIG. 30 discloses SEQ ID NOS 99-107, 106, 107, 107, 107, 107, 107-112, respectively, in order of appearance.

FIG. 31 shows a list of genes differentially expressed in CD33 deleted cells. Genes whose expression is low in CD33 deleted cells compared to CD33 wild type cells are indicated by negative sign.

FIGS. 32A-32C show that GO targeted immunotherapy clears primary AML and rescue CD33^{Del} cells. FIG. 32A: Schematic experimental design: 0.5 million primary AML cells were injected in NSGS mice on day 1. Once Minimal Residual Disease assessed, the treated mice group received chronic dose of GO (1 μg every 10 days). 10 days after first GO injection of the treated group, mice were transplanted with 0.5 million CD34⁺CD33^{Del} cells. FIG. 32B, left panel: AML (Minimal residual Disease) burden assessed by flow cytometry of bone marrow aspirate prior to treatment. Leukemia cells were gated on Ter119⁻hCD45⁺hCD33⁺. Right panels: AML burden and hCD45⁺hCD33^{Del} engraftment in BM aspirate after GO chronic treatment. FIG. 23B (continued): Hematopoietic repopulation (myeloid/lymphoid progenitors and mature cells) of CD34⁺CD33^{Del} cells gated on Ter119⁻Ly5⁻/H2kd⁻, hCD45⁺, hCD33⁺. FIG. 32C. Survival curve and analysis of whole bone marrow (BM), spleen, and peripheral blood (PB) of control group at the time of death (solid line, untreated; dashed line, treated).

FIGS. 33A-33D show cells made deficient in CD33 and CLL-1 can successfully engraft. FIG. 33A is a series of flow cytometry plots showing CD33 and CLL-1 levels can be reduced individually or in combination. FIG. 33B is a series of flow cytometry plots showing CD33 and CLL-1 levels in cells allowed to engraft in mice. FIG. 33C and 33D: frequency within the hCD45⁺ cell population of the indicated cell types in while bone marrow samples (FIG. 33C) or spleen samples (FIG. 34D). From left to right, each set of four bars represents the following cell types: CD34^{+WT} (circles); CD34⁺CD33^{Del} (squares), CD34⁺CLL1^{Del} (triangles) and CD34⁺CD33^{Del}CLL1^{Del} (inverted triangles).

### DETAILED DESCRIPTION OF DISCLOSURE

Cancer immunotherapies targeting antigens present on the cell surface of a cancer cell is particularly challenging when the target antigen is also present on the cell surface of normal, non-cancer cells that are required or critically involved in the development and/or survival of the subject. Targeting these antigens may lead to deleterious effects in the subject due to cytotoxic effects of the immunotherapy toward such cells in addition to the cancer cells.

The methods, nucleic acids, and cells described herein allow for targeting of antigens (e.g., type 1 or type 2 antigens) that are present not only on cancer cells but also cells critical for the development and/or survival of the subject. The method involves: (1) reducing the number of cells carrying the target lineage-specific cell-surface antigen using an agent that targets such an antigen; and (2) replacement of the normal cells (e.g., non-cancer cells) that present the antigen and thus can be killed due to administration of the agent with hematopoietic cells that are deficient for the lineage-specific cell-surface antigen. The methods described herein can maintain surveillance for target cells, including cancer cells, that express a lineage-specific cell-surface antigen of interest and also maintain the population of non-cancer cells expressing the lineage-specific antigen, which may be critical for development and/or survival of the subject.

Accordingly, described herein are the co-use of immune cells expressing chimeric receptors comprising an antigen-binding fragment that targets a lineage-specific cell-surface antigen (e.g., CD33) and hematopoietic cells such as hematopoietic stem cells (HSCs) or hematopoietic progenitor cells (HPCs) that are deficient in the lineage-specific cell-surface antigen for treating a hematopoietic malignancy. Also provided herein are the chimeric receptors, nucleic acids encoding such, vectors comprising such, and immune cells (e.g., T cells) expressing such a chimeric receptor. The present disclosure also provides genetically engineered hematopoietic cells that are deficient in a lineage-specific antigen such as those described herein, as well as methods (e.g., genome editing methods) for making such.

### Definitions

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. Parts of the vectors can be naturally-occurring. The non-naturally occurring recombinant expression vectors of the present disclosure can comprise any type of nucleotides, including, but not limited to DNA and RNA, which can be single-stranded or double-stranded, synthesized or obtained in part from natural sources, and which can contain natural, non-natural or altered nucleotides.

“Transfection,” “transformation,” or “transduction,” as used herein, refer to the introduction of one or more exogenous polynucleotides into a host cell by using physical or chemical methods.

“Antibody,” “fragment of an antibody,” “antibody fragment,” “functional fragment of an antibody,” or “antigen-binding portion” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

The present antibodies or antigen-binding portions can specifically bind with a dissociation constant (KD) of less than about 10⁻⁷ M, less than about 10⁻⁸ M, less than about 10⁻⁹ M, less than about 10⁻¹⁰ less than about 10⁻¹¹ M, or less than about 10⁻¹² M. Affinities of the M, antibodies according to the present disclosure can be readily determined using conventional techniques (see, e.g., Scatchard et al., Ann. N.Y. Acad. Sci. (1949) 51:660; and U.S. Pat. Nos. 5,283,173, 5,468,614, or the equivalent).

The terms “chimeric receptor,” “Chimeric Antigen Receptor,” or alternatively a “CAR” are used interchangeably throughout and refer to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. Lee et al., Clin. Cancer Res. (2012) 18(10):2780; Jensen et al., Immunol Rev. (2014) 257(1):127; www.cancer.gov/about-cancer/treatment/research/car-t-cells. In one embodiment, the stimulatory molecule is the zeta chain associated with the T cell receptor complex. In one aspect, the cytoplasmic signaling domain further comprises one or more functional signaling domains derived from at least one costimulatory molecule as defined below. The costimulatory molecule may also be 4-1BB (i.e., CD137), CD27 and/or CD28 or fragments of those molecules. In another aspect, the CAR comprises a chimeric fusion protein comprising an extracellular antigen recognition domain, a transmembrane domain and an intracellular signaling domain comprising a functional signaling domain derived from a stimulatory molecule. The CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. Alternatively, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. The CAR can also comprise a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. The antigen recognition moiety of the CAR encoded by the nucleic acid sequence can contain any lineage specific, antigen-binding antibody fragment. The antibody fragment can comprise one or more CDRs, the variable region (or portions thereof), the constant region (or portions thereof), or combinations of any of the foregoing.

The term “signaling domain” refers to the functional portion of a protein which acts by transmitting information within the cell to regulate cellular activity via defined signaling pathways by generating second messengers or functioning as effectors by responding to such messengers.

The term “zeta” or alternatively “zeta chain”, “CD3-zeta” or “TCR-zeta” is defined as the protein provided as GenBank accession numbers NP_932170, NP_000725, or XP_011508447; or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation.

The term “genetically engineered” or “genetically modified” refers to cells being manipulated by genetic engineering, for example by genome editing. That is, the cells contain a heterologous sequence which does not naturally occur in said cells. Typically, the heterologous sequence is introduced via a vector system or other means for introducing nucleic acid molecules into cells including liposomes. The heterologous nucleic acid molecule may be integrated into the genome of the cells or may be present extra-chromosomally, e.g., in the form of plasmids.

The term also includes embodiments of introducing genetically engineered, isolated CAR polypeptides into the cell.

The term “autologous” refers to any material derived from the same individual to whom it is later to be re-introduced into the same individual.

The term “allogeneic” refers to any material derived from a different animal of the same species as the individual to whom the material is introduced. Two or more individuals are said to be allogeneic to one another when the genes at one or more loci are not identical.

The term “cell lineage” refers to cells with a common ancestry and developing from the same type of identifiable cell into specific identifiable/functioning cells. The cell lineages used herein include, but are not limited to, respiratory, prostatic, pancreatic, mammary, renal, intestinal, neural, skeletal, vascular, hepatic, hematopoietic, muscle or cardiac cell lineages.

The term “inhibition” when used in reference to gene expression or function of a lineage specific antigen refers to a decrease in the level of gene expression or function of the lineage specific antigen, where the inhibition is a result of interference with gene expression or function. The inhibition may be complete, in which case there is no detectable expression or function, or it may be partial. Partial inhibition can range from near complete inhibition to a near absence of inhibition. By eliminating particular target cells, CAR T cells may effectively inhibit the overall expression of particular cell lineage.

Cells such as hematopoietic cells that are “deficient in a lineage-specific antigen” refers to cells having a substantially reduced expression level of the lineage-specific antigen as compared with their naturally-occurring counterpart, e.g., endogenous hematopoietic cells of the same type, or cells that do not express the lineage-specific antigen, i.e., not detectable by a routine assay such as FACS. In some instances, the express level of a lineage-specific antigen of cells that are “deficient in the antigen” can be lower than about 40% (e.g., 30%, 20%, 15%, 10%, 5% or lower) of the expression level of the same lineage-specific antigen of the naturally-occurring counterpart. As used herein, the term “about” refers to a particular value +/−5%. For example, an expression level of about 40% may include any amount of expression between 35%-45%.

### Agents Targeting Llineage-Specific Cell-Surface Antigens

Aspects of the disclosure provide agents (e.g., agents that target CD33, e.g., wherein the agent comprises an antigen-binding fragment that binds CD33) targeting a lineage-specific cell-surface antigen, for example on a target cancer cell. Such an agent may comprise an antigen-binding fragment that binds and targets the lineage-specific cell-surface antigen. In some instances, the antigen-binding fragment can be a single chain antibody (scFv) specifically binding to the lineage-specific antigen.

A. Lineage-Specific Cell-Surface Antigens

As used herein, the terms “lineage-specific cell-surface antigen” and “cell-surface lineage-specific antigen” may be used interchangeably and refer to any antigen that is sufficiently present on the surface of a cell and is associated with one or more populations of cell lineage(s). For example, the antigen may be present on one or more populations of cell lineage(s) and absent (or at reduced levels) on the cell-surface of other cell populations.

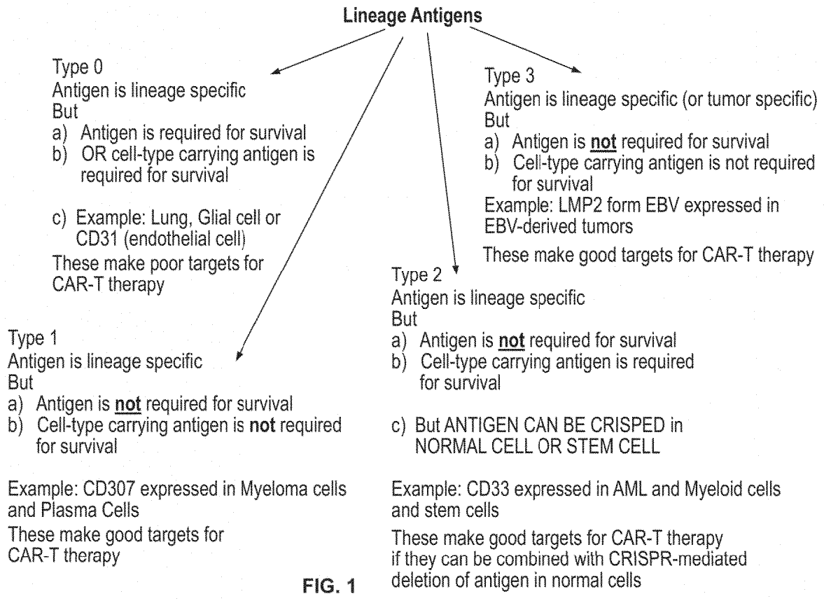

In general, lineage-specific cell-surface antigens can be classified based on a number of factors such as whether the antigen and/or the populations of cells that present the antigen are required for survival and/or development of the host organism. A summary of exemplary types of lineage-specific antigens is provide in Table 1 below. See also FIG. 1.

TABLE 1
Classification of Lineage Specific Antigens

| Type of Lineage | Characteristics of the Lineage Specific |
|---|---|
| Specific Antigen | Antigen |
| Type 0 | a) antigen is required for survival of an |
| | organism |
| | and |
| | b) cell type carrying type 0 antigen is |
| | required for survival of an organism and is |
| | not unique to a tumor, or tumor-associated |
| | virus |
| Type 1 | a) antigen is not required for survival |
| | of an organism |
| | and |
| | b) cell type carrying type 1 antigen is |
| | not required for survival of an organism |
| Type 2 | a) antigen is not required for survival |
| | of an organism |
| | and |
| | b) cell type carrying type 2 antigen is |
| | required for the survival of an organism |
| Type 3 | a) antigen is not required for the |
| | survival of an organism |
| | and |
| | b) cell type carrying antigen is not |
| | required for survival of an organism |
| | c) The antigen is unique to a tumor, or |
| | a tumor associated virus |
| | An example is the LMP-2 antigen in EBV |
| | infected cells, including EBV infected |
| | tumor cells (Nasopharyngeal carcinoma |
| | and Burkitts Lymphoma) |

As shown in Table 1 and FIG. 1, type 0 lineage-specific cell-surface antigens are necessary for the tissue homeostasis and survival, and cell types carrying type 0 lineage-specific cell-surface antigen may be also necessary for survival of the subject. Thus, given the importance of type 0 lineage-specific cell-surface antigens, or cells carrying type 0 lineage-specific cell-surface antigens, in homeostasis and survival, targeting this category of antigens may be challenging using conventional CAR T cell immunotherapies, as the inhibition or removal of such antigens and cell carrying such antigens may be detrimental to the survival of the subject. Consequently, lineage-specific cell-surface antigens (such as type 0 lineage-specific antigens) and/or the cell types that carry such antigens may be required for the survival, for example because it performs a vital non-redundant function in the subject, then this type of lineage specific antigen may be a poor target for CAR T cell based immunotherapy.

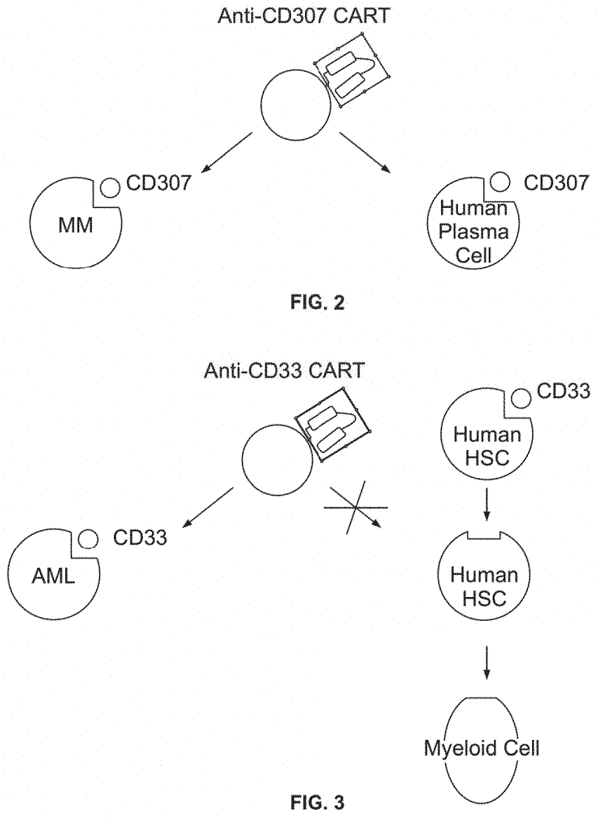

In contrast to type 0 antigens, type 1 cell-surface lineage-specific antigens and cells carrying type 1 cell-surface lineage-specific antigens are not required for tissue homeostasis or survival of the subject. Targeting type 1 cell-surface lineage-specific antigens is not likely to lead to detrimental consequences in the subject. For example, a CAR T cell engineered to target CD307, a type 1 antigen expressed uniquely on both normal plasma cells and multiple myeloma (MM) cells would lead to elimination of both cell types (FIG. 2) (Elkins et al., Mol Cancer Ther. 10:2222 (2012)). However, since the plasma cell lineage is expendable for the survival of the organism, CD307 and other type 1 lineage specific antigens are antigens that are suitable for CAR T cell based immunotherapy. Lineage specific antigens of type 1 class may be expressed in a wide variety of different tissues, including, ovaries, testes, prostate, breast, endometrium, and pancreas. In some embodiments, the agent targets a cell-surface lineage-specific antigen that is a type 1 antigen.

Targeting type 2 antigens presents a significant difficulty as compared to type 1 antigens. Type 2 antigens are those characterized where: (1) the antigen is dispensable for the survival of an organism (i.e., is not required for the survival), and (2) the cell lineage carrying the antigen is indispensable for the survival of an organism (i.e., the particular cell lineage is required for the survival). For example, CD33 is a type 2 antigen expressed in both normal myeloid cells as well as in Acute Myeloid Leukemia (AML) cells (Dohner et al., NEJM 373:1136 (2015)). As a result, a CAR T cell engineered to target CD33 antigen could lead to the killing of both normal as well as AML cells, which may be incompatible with survival of the subject (FIG. 3). In some embodiments, the agent targets a cell-surface lineage-specific antigen that is a type 2 antigen.

A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In some embodiments, the cell-surface lineage-specific antigen that is targeted using the methods and cells described herein is a cell-surface lineage-specific antigen of leukocytes or a subpopulation of leukocytes. In some embodiments, the cell-surface lineage-specific antigen is an antigen that is associated with myeloid cells. In some embodiments, the cell-surface lineage-specific antigen is a cluster of differentiation antigens (CDs). Examples of CD antigens include, without limitation, CD1a, CD1b, CD1c, CD1d, CD1e, CD2, CD3, CD3d, CD3e, CD3g, CD4, CDS, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD851, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, 306, CD307a, CD307b, CD307c, D307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 and CD363. See www.bdbiosciences.com/documents/BD_Reagents_CDMarkerHuman_Poster.pdf.

In some embodiments, the cell-surface lineage-specific antigen is CD19, CD20, CD11, CD123, CD56, CD34, CD14, CD33, CD66b, CD41, CD61, CD62, CD235a, CD146, CD326, LMP2, CD22, CD52, CD10, CD3/TCR, CD79/BCR, and CD26. In some embodiments, the cell-surface lineage-specific antigen is CD33.

Alternatively or in addition, the cell-surface lineage-specific antigen may be a cancer antigen, for example a cell-surface lineage-specific antigen that is differentially present on cancer cells. In some embodiments, the cancer antigen is an antigen that is specific to a tissue or cell lineage. Examples of cell-surface lineage-specific antigen that are associated with a specific type of cancer include, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen. In some embodiments, the cell-surface antigen CD33 and is associated with AML cells.

B. Antigen-Binding Fragment

Any antibody or an antigen-binding fragment thereof (e.g., which binds CD33) can be used for constructing the agent that targets a lineage-specific cell-surface antigen as described herein. Such an antibody or antigen-binding fragment can be prepared by a conventional method, for example, using hybridoma technology or recombinant technology.

For example, antibodies specific to a lineage-specific antigen of interest can be made by conventional hybridoma technology. The lineage-specific antigen, which may be coupled to a carrier protein such as KLH, can be used to immunize a host animal for generating antibodies binding to that complex. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a lineage-specific antigen. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOLI, or R1N=C=NR, where R and R1 are different alkyl groups, can yield a population of antibodies (e.g., monoclonal antibodies).

If desired, an antibody of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the lineage-specific antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab')2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a lineage-specific antigen can be identified from the library following routine procedures. Positive clones can be subjected to further screening to identify those that bind lineage-specific antigen.

In some instances, lineage-specific antigen of interest is CD33 and the antigen-binding fragment specifically binds CD33, for example, human CD33. Amino acid and nucleic acid sequences of an exemplary heavy chain variable region and light chain variable region of an anti-human CD33 antibody are provided below. The CDR sequences are shown in boldface and underlined in the amino acid sequences.

Amino acid sequence of anti-CD33 Heavy Chain
Variable Region
(SEQ ID NO: 12)
QVQLQQPGAEVVKPGASVKMSCKASGYTFTSYYIHWIKQTPGQGLEWVG
VIYPGNDDISYNQKFQGKATLTADKSSTTAYMQLSSLTSEDSAVYYCAR
EVRLRYFDVWGQGTTVTVSS
Nucleic acid sequence of anti-CD33 Heavy Chain
Variable Region
(SEQ ID NO: 2)
CAGGTGCAGCTGCAGCAGCCCGGCGCCGAGGTGGTGAAGCCCGGCGCCA
GCGTGAAGATGAGCTGCAAGGCCAGCGGCTACACCTTCACCAGCTACTA
CATCCACTGGATCAAGCAGACCCCCGGCCAGGGCCTGGAGTGGGTGGGC
GTGATCTACCCCGGCAACGACGACATCAGCTACAACCAGAAGTTCCAGG
GCAAGGCCACCCTGACCGCCGACAAGAGCAGCACCACCGCCTACATGCA
GCTGAGCAGCCTGACCAGCGAGGACAGCGCCGTGTACTACTGCGCCAGG
GAGGTGAGGCTGAGGTACTTCGACGTGTGGGGCCAGGGCACCACCGTGA
CCGTGAGCAGC
Amino acid sequence of anti-CD33 Light Chain
Variable Region
(SEQ ID NO: 13)
EIVLTQSPGSLAVSPGERVTMSCKSSQSVFFSSSQKNYLAWYQQIPGQS
PRLLIYWASTRESGVPDRFTGSGSGTDFTLTISSVQPEDLAIYYCHQYL
SSRTFGQGTKLEIKR
Nucleic acid sequence of anti-CD33 Heavy Chain
Variable Region
(SEQ ID NO: 1)
GAGATCGTGCTGACCCAGAGCCCCGGCAGCCTGGCCGTGAGCCCCGGCG
AGAGGGTGACCATGAGCTGCAAGAGCAGCCAGAGCGTGTTCTTCAGCAG
CAGCCAGAAGAACTACCTGGCCTGGTACCAGCAGATCCCCGGCCAGAGC
CCCAGGCTGCTGATCTACTGGGCCAGCACCAGGGAGAGCGGCGTGCCCG
ACAGGTTCACCGGCAGCGGCAGCGGCACCGACTTCACCCTGACCATCAG
CAGCGTGCAGCCCGAGGACCTGGCCATCTACTACTGCCACCAGTACCTG
AGCAGCAGGACCTTCGGCCAGGGCACCAAGCTGGAGATCAAGAGG

The anti-CD33 antibody binding fragment for use in constructing the agent that targets CD33 as described herein may comprise the same heavy chain and/or light chain CDR regions as those in SEQ ID NO:12 and SEQ ID NO:13. Such antibodies may comprise amino acid residue variations in one or more of the framework regions. In some instances, the anti-CD33 antibody fragment may comprise a heavy chain variable region that shares at least 70% sequence identity (e.g., 75%, 80%, 85%, 90%, 95%, or higher) with SEQ ID NO:12 and/or may comprise a light chain variable region that shares at least 70% sequence identity (e.g., 75%, 80%, 85%, 90%, 95%, or higher) with SEQ ID NO:13.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the present disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

C. Immune Cells Expressing Chimeric Receptors

In some embodiments, the agent that targets a lineage-specific cell-surface antigen as described herein is an immune cell that expresses a chimeric receptor, which comprises an antigen-binding fragment (e.g., a single-chain antibody) capable of binding to the lineage-specific antigen (e.g., CD33). Recognition of a target cell (e.g., a cancer cell) having the lineage-specific antigen on its cell surface by the antigen-binding fragment of the chimeric receptor transduces an activation signal to the signaling domain(s) (e.g., co-stimulatory signaling domain and/or the cytoplasmic signaling domain) of the chimeric receptor, which may activate an effector function in the immune cell expressing the chimeric receptor.

As used herein, a chimeric receptor refers to a non-naturally occurring molecule that can be expressed on the surface of a host cell and comprises an antigen-binding fragment that binds to a cell-surface lineage-specific antigen. In general, chimeric receptors comprise at least two domains that are derived from different molecules. In addition to the antigen-binding fragment described herein, the chimeric receptor may further comprise one or more of a hinge domain, a transmembrane domain, at least one co-stimulatory domain, and a cytoplasmic signaling domain. In some embodiments, the chimeric receptor comprises from N terminus to C terminus, an antigen-binding fragment that binds to a cell-surface lineage-specific antigen, a hinge domain, a transmembrane domain, and a cytoplasmic signaling domain. In some embodiments, the chimeric receptor further comprises at least one co-stimulatory domain.

In some embodiments, the chimeric receptors described herein comprise a hinge domain, which may be located between the antigen-binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the antigen-binding fragment relative to another domain of the chimeric receptor can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14,15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α or CD28α. In some embodiments, the hinge domain is a portion of the hinge domain of CD8α, e.g., a fragment containing at least 15 (e.g., 20, 25, 30, 35, or 40) consecutive amino acids of the hinge domain of CD8α or CD28α.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Also within the scope of the present disclosure are chimeric receptors comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyₓSer)ₙ linker (SEQ ID NO: 74), wherein x and n, independently can be an integer between 3 and 12, including 3, 4, 5, 6, 7, 8, 9, 10, 11, 12, or more.

Additional peptide linkers that may be used in a hinge domain of the chimeric receptors described herein are known in the art. See, e.g., Wriggers et al. rent Trends in Peptide Science 2005) 80(6): 736-746 and PCT Publication WO 2012/088461.

In some embodiments, the chimeric receptors described herein may comprise a transmembrane domain. The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, the transmembrane domain may be a synthetic, non-naturally occurring protein segment, e.g., a hydrophobic protein segment that is thermodynamically stable in a cell membrane.

Transmembrane domains are classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). In some embodiments, the transmembrane domain is a single-pass transmembrane domain. In some embodiments, the transmembrane domain is a single-pass transmembrane domain that orients the N terminus of the chimeric receptor to the extracellular side of the cell and the C terminus of the chimeric receptor to the intracellular side of the cell. In some embodiments, the transmembrane domain is obtained from a single pass transmembrane protein. In some embodiments, the transmembrane domain is of CD8a. In some embodiments, the transmembrane domain is of CD28. In some embodiments, the transmembrane domain is of ICOS.

In some embodiments, the chimeric receptors described herein comprise one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

In some embodiments, the chimeric receptor comprises more than one (at least 2, 3, 4, or more) co-stimulatory signaling domains. In some embodiments, the chimeric receptor comprises more than one co-stimulatory signaling domains obtained from different costimulatory proteins. In some embodiments, the chimeric receptor does not comprise a co-stimulatory signaling domain.

In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domain is selected based on factors such as the type of the immune cells in which the chimeric receptors would be expressed (e.g., primary T cells, T cell lines, NK cell lines) and the desired immune effector function (e.g., cytotoxicity). Examples of co-stimulatory signaling domains for use in the chimeric receptors can be the cytoplasmic signaling domain of co-stimulatory proteins, including, without limitation, CD27, CD28, 4-1BB, OX40, CD30, Cd40, PD-1, ICOS, lymphocyte function-associated antigen-1 (LFA-1), CD2, CD7, LIGHT, NKG2C, B7-H3. In some embodiments, the co-stimulatory domain is derived from 4-1BB, CD28, or ICOS. In some embodiments, the costimulatory domain is derived from CD28 and chimeric receptor comprises a second co-stimulatory domain from 4-1BB or ICOS.

In some embodiments, the costimulatory domain is a fusion domain comprising more than one costimulatory domain or portions of more than one costimulatory domains. In some embodiments, the costimulatory domain is a fusion of costimulatory domains from CD28 and ICOS.

In some embodiments, the chimeric receptors described herein comprise a cytoplasmic signaling domain. Any cytoplasmic signaling domain can be used in the chimeric receptors described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ζ.

Exemplary chimeric receptors are provided in Tables 2 and 3 below.

TABLE 2
Exemplary components of a chimeric receptor

| Chimeric receptor component | Amino acid sequence |
|---|---|
| Antigen-binding fragment | Light chain-GSTSSGSGKPGSGEGSTKG |
| | (SEQ ID NO: 14)-Heavy chain |
| CD28 costimulatory domain | IEVMYPPPYLDNEKSNGTIIHVKGKHLCPSP |
| | LFPGPSKPFWVLVVVGGVLACYSLLVTVA |
| | FIIFWVRSKRSRLLHSDYMNMTPRRPGPTR |
| | KHYQPYAPPRDFAAYRS (SEQ ID NO: 6) |
| ICOS costimulatory domain (boldface), | LSIFDPPPFKVTLTGGYLHIYESQLCCQLKF |
| ICOS transmembrane domain (italics) | WLPIGCAAFVVVCILGCILICWLTKKKYSSS |
| and a portion of the extracellular | VHDPNGEYMFMRAVNTAKKSRLTDVTL |
| domain of ICOS (underlined) | (SEQ ID NO: 7) |
| ICOS costimulatory domain | CWLTKKKYSSSVHDPNGEYMFMRAVNTA |
| | KKSRLTDVT (SEQ ID NO: 47) |
| CD28/ICOS chimera (the ICOS portion | IEVMYPPPYLDNEKSNGTIIHVKGKHLCPSPL |
| shown in underline) including the hinge | FPGPSKPFWVLVVVGGVLACYSLLVTVA |
| domain (italics) and transmembrane | FIIFWVRSKRSRLLHSDYMFMRAVNTAKK |
| domain (bold) from CD28 | SRLTDVTL (SEQ ID NO: 8) |
| CD3ζ cytoplasmic signaling domain | RVKFSRSADAPAYQQGQNQLYNELNLGRR |
| | EEYDVLDKRRGRDPEMGGKPQRRKNPQE |
| | GLYNELQKDKMAEAYSEIGMKGERRRGK |
| | GHDGLYQGLSTATKDTYDALHMQALPPR |
| | (SEQ ID NO: 15) |

The nucleic acid sequence of exemplary components for construction of a chimeric receptor are provided below.

CD28 intracellular signaling domain-DNA-Human
(SEQ ID NO: 3)
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCCTTTTGGGTGCTGGTGGTGGTTGGTGGAGTC
CTGGCTTGCTATAGCTTGCTAGTAACAGTGGCCTTTATTATTTTCTGGG
TGAGGAGTAAGAGGAGCAGGCTCCTGCACAGTGACTACATGAACATGAC
TCCCCGCCGCCCCGGGCCCACCCGCAAGCATTACCAGCCCTATGCCCCA
CCACGCGACTTCGCAGCCTATCGCTCCAGAGTGAAGTTCAGCAGGAGCG
CAGACGCCCCCGCGTACCAGCAGGGCCAGAACCAGCTCTATAACGAGCT
CAATCTAGGACGAAGAGAGGAGTACGATGTTTTGGACAAGAGACGTGGC
CGGGACCCTGAGATGGGGGGAAAGCCGAGAAGGAAGAACCCTCAGGAAG
GCCTGTACAATGAACTGCAGAAAGATAAGATGGCGGAGGCCTACAGTGA
GATTGGGATGAAAGGCGAGCGCCGGAGGGGCAAGGGGCACGATGGCCTT
TACCAGGGTCTCAGTACAGCCACCAAGGACACCTACGACGCCCTTCACA
TGCAGGCCCTGCCCCCTCGC
ICOS intracellular signaling domain-DNA-Human
(SEQ ID NO: 4)
CTATCAATTTTTGATCCTCCTCCTTTTAAAGTAACTCTTACAGGAGGAT
ATTTGCATATTTATGAATCACAACTTTGTTGCCAGCTGAAGTTCTGGTT
ACCCATAGGATGTGCAGCCTTTGTTGTAGTCTGCATTTTGGGATGCATA
CTTATTTGTTGGCTTACAAAAAAGAAGTATTCATCCAGTGTGCACGACC
CTAACGGTGAATACATGTTCATGAGAGCAGTGAACACAGCCAAAAAATC
TAGACTCACAGATGTGACCCTAAGAGTGAAGTTCAGCAGGAGCGCAGAC
GCCCCCGCGTACCAGCAGGGCCAGAACCAGCTCTATAACGAGCTCAATC
TAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTG
TACAATGAACTGCAGAAAGATAAGATGGCGGAGGCCTACAGTGAGATTG
GGATGAAAGGCGAGCGCCGGAGGGGCAAGGGGCACGATGGCCTTTACCA
GGGTCTCAGTACAGCCACCAAGGACACCTACGACGCCCTTCACATGCAG
GCCCTGCCCCCTCGC
CD28/ICOS COSTIMULATORY SIGNALING REGION-DNA-
Human
(SEQ ID NO: 5)
ATTGAAGTTATGTATCCTCCTCCTTACCTAGACAATGAGAAGAGCAATG
GAACCATTATCCATGTGAAAGGGAAACACCTTTGTCCAAGTCCCCTATT
TCCCGGACCTTCTAAGCCCTTTTGGGTGCTGGTGGTGGTTGGTGGAGTC
CTGGCTTGCTATAGCTTGCTAGTAACAGTGGCCTTTATTATTTTCTGGG
TGAGGAGTAAGAGGAGCAGGCTCCTGCACAGTGACTACATGTTCATGAG
AGCAGTGAACACAGCCAAAAAATCTAGACTCACAGATGTGACCCTAAGA
GTGAAGTTCAGCAGGAGCGCAGACGCCCCCGCGTACCAGCAGGGCCAGA
ACCAGCTCTATAACGAGCTCAATCTAGGACGAAGAGAGGAGTACGATGT
TTTGGACAAGAGACGTGGCCGGGACCCTGAGATGGGGGGAAAGCCGAGA
AGGAAGAACCCTCAGGAAGGCCTGTACAATGAACTGCAGAAAGATAAGA
TGGCGGAGGCCTACAGTGAGATTGGGATGAAAGGCGAGCGCCGGAGGGG
CAAGGGGCACGATGGCCTTTACCAGGGTCTCAGTACAGCCACCAAGGAC
ACCTACGACGCCCTTCACATGCAGGCCCTGCCCCCTCGC

In some embodiments, the nucleic acid sequence encodes an antigen binding fragment that binds to CD33 and comprises a heavy chain variable region which has the same CDRs as the CDRs in SEQ ID NO: 12 and a light chain variable region which has the same CDRs as the CDRs in SEQ ID NO: 13. In some embodiments, the antigen-binding fragment comprises a heavy chain variable region as provided by SEQ ID NO: 12 and a light chain variable region as provided by SEQ ID NO: 13. In some embodiments, the chimeric receptor further comprises at least a transmembrane domain and a cytoplasmic signaling domain. In some embodiments, the chimeric receptor further comprises a hinge domain and/or a co-stimulatory signaling domain.

Table 3 provides exemplary chimeric receptors described herein. The exemplary constructs have from N-terminus to C-terminus, the antigen-binding fragment, the transmembrane domain, and a cytoplasmic signaling domain. In some examples, the chimeric receptor further comprises a hinge domain located between the antigen-binding fragment and the transmembrane domain. In some example, the chimeric receptor further comprises one or more co-stimulatory domains., which may be located between the transmembrane domain and the cytoplasmic signaling domain.

TABLE 3
Exemplary chimeric receptors

| | | Antigen- | | | | | |
|---|---|---|---|---|---|---|---|
| | | binding | | Trans- | | | |
| | | fragment | Hinge | membrane | Signaling | Signaling | Signaling |
| Constructs | Vector | specificity | domain | domain | domain 1 | domain 2 | domain 3 |
| CART 1 | HIVzs-Gfp | CD33 | CD8α | CD8 | 4-1BB | CD3ζ | None |
| (SEQ ID | | | | | | | |
| NO: 20) | | | | | | | |
| CART 2 | HIVzs-Gfp | CD33 | CD8α | CD28 | CD28 | CD3ζ | None |
| (SEQ ID | | | | | | | |
| NO: 21) | | | | | | | |
| CART 3 | HIVzs-Gfp | CD33 | CD8α | CD28 | CD28 | 4-1BB | CD3ζ |
| (SEQ ID | | | | | | | |
| NO: 22) | | | | | | | |
| CART 8 | HIVzs-Gfp | CD33 | CD8α | ICOS | ICOS | 4-1BB | CD3ζ |
| (SEQ ID | | | | | | | |
| NO: 23) | | | | | | | |
| CART 4dual | HIVzs-dT | CD19 | CD8α | CD28 | CD3ζ | — | — |
| (SEQ ID | | | | | | | |
| NO: 24) | | | | | | | |
| CART 5dual | HIVzs-Gfp | CD33 | CD8α | CD28 | CD28 | — | — |
| (SEQ ID | | | | | | | |
| NO: 25) | | | | | | | |
| CART 6 | HIVzs-dT | CD19 | CD8α | CD28 | CD28 | CD3ζ | — |
| (SEQ ID | | | | | | | |
| NO: 26) | | | | | | | |
| CART 7 | HIVzs-Gfp | CD33 | CD28hge | CD28 | CD28 | CD3ζ | — |
| (SEQ ID | | | | | | | |
| NO: 27) | | | | | | | |

Amino acid sequences of the example chimeric receptors listed in Table 3 above are provided below:

CART1 amino acid sequence
(SEQ ID NO: 20)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSSALSNSIMYFSHFVPVFLPAKPTT
TPAPRPPTPAPTIASQPLSLRPEASRPAAGGAVHTRGLDIYIWAPLAGTCGVLLLSLVIT
KRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CART2 amino acid sequence
(SEQ ID NO: 21)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSALSNSIMYFSHFVPVFLPAKPTTT
PAPRPPTPAPTIASQPLSLRPEASRPAAGGAVHTRGLDKPFWVLVVVGGVLACYSLLV
TVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
CART3 amino acid sequence
(SEQ ID NO: 22)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSSALSNSIMYFSHFVPVFLPAKPTT
TPAPRPPTPAPTIASQPLSLRPEASRPAAGGAVHTRGLDKPFWVLVVVGGVLACYSLL
VTVAFIIFWVRSKRSRLLHSDYMNMTPRRPGPTRKHYQPYAPPRDFAAYRSKRGRKK
LLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGM
KGERRRGKGHDGLYQGLSTATKDTYDALHMQALPPR
CART8 amino acid sequence
(SEQ ID NO: 23)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSSALSNSIMYFSHFVPVFLPAKPTT
TPAPRPPTPAPTIASQPLSLRPEASRPAAGGAVHTRGLDFWLPIGCAAFVVVCILGCILI
CWLTKKKYSSSVHDPNGEYMFMRAVNTAKKSRLTDVTLTKRGRKKLLYIFKQPFMR
PVQTTQEEDGCSCRFPEEEEGGCELRVKFSRSADAPAYQQGQNQLYNELNLGRREEY
DVLDKRRGRDPEMGGKPRRKNPQEGLYNELQKDKMAEAYSEIGMKGERRRGKGHD
GLYQGLSTATKDTYDALHMQALPPR
CART4dual amino acid sequence
(SEQ ID NO: 24)
MWLQSLLLLGTVACSISIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGG
TKLEIGSTSGSGKPGSGEGSTKGLQESGPGLVAPSQSLSVTCTVSGVSLPDYGVSWIR
QPPRKGLEWLGVIWGSETTYYNSALKSRLTIIKDNSKSQVFLKMNSLQTDDTAIYYC
AKHYYYGGSYAMDYWGQGTSVTVSALSNSIMYFSHFVPVFLPAKPTTTPAPRPPTPA
PTIASQPLSLRPEASRPAAGGAVHTRGLDKPFWVLVVVGGVLACYSLLVTVAFIIFWV
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALP
PR
CART5dual amino acid sequence
(SEQ ID NO: 25)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSSALSNSIMYFSHFVPVFLPAKPTT
TPAPRPPTPAPTIASQPLSLRPEASRPAAGGAVHTRGLDKPFWVLVVVGGVLACYSLL
VTVAFIIFWVRSKRSRLLHSDYMNMTPRRPGPTRKHYQPYAPPRDFAAYRS
CART6 amino acid sequence
(SEQ ID NO: 26)
MWLQSLLLLGTVACSISIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGG
TKLEIGSTSGSGKPGSGEGSTKGLQESGPGLVAPSQSLSVTCTVSGVSLPDYGVSWIR
QPPRKGLEWLGVIWGSETTYYNSALKSRLTIIKDNSKSQVFLKMNSLQTDDTAIYYC
AKHYYYGGSYAMDYWGQGTSVTVSALSNSIMYFSHFVPVFLPAKPTTTPAPRPPTPA
PTIASQPLSLRPEASRPAAGGAVHTRGLDKPFWVLVVVGGVLACYSLLVTVAFIIFWV
RSKRSRLLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CART7 amino acid sequence
(SEQ ID NO: 27)
MWLQSLLLLGTVACSISEIVLTQSPGSLAVSPGERVTMSCKSSQSVFFSSSQKNYLAW
YQQIPGQSPRLLIYWASTRESGVPDRFTGSGSGTDFTLTISSVQPEDLAIYYCHQYLSS
RTFGQGTKLEIKRGSTSGSGKPGSGEGSTKGQVQLQQPGAEVVKPGASVKMSCKASG
YTFTSYYIHWIKQTPGQGLEWVGVIYPGNDDISYNQKFQGKATLTADKSSTTAYMQL
SSLTSEDSAVYYCAREVRLRYFDVWGQGTTVTVSSIEVMYPPPYLDNEKSNGTIIHVK
GKHLCPSPLFPGPSKPFWVLVVVGGVLACYSLLVTVAFIIFWVRSKRSRLLHSDYMN
MTPRRPGPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMGERRRGKG
HDGLYQGLSTATKDTYDALHMQALPPR

Nucleic acid sequences of the example chimeric receptors listed in Table 3 above are provided below:

CART1 nucleic acid sequence
(SEQ ID NO: 38)
GGTGTCGTGAGCGGCCGCTGAACTGGCCACCATGTGGCTGCAGTCTCTGCTGCTG
CTGGGCACCGTGGCCTGTAGCATCAGCGAGATCGTGCTGACCCAGAGCCCTGGCT
CTCTGGCTGTGTCTCCTGGCGAGCGCGTGACCATGAGCTGCAAGAGCAGCCAGAG
CGTGTTCTTCAGCAGCTCCCAGAAGAACTACCTGGCCTGGTATCAGCAGATCCCC
GGCCAGAGCCCCAGACTGCTGATCTACTGGGCCAGCACCAGAGAAAGCGGCGTG
CCCGATAGATTCACCGGCAGCGGCTCTGGCACCGACTTCACCCTGACAATCAGCA
GCGTGCAGCCCGAGGACCTGGCCATCTACTACTGCCACCAGTACCTGAGCAGCCG
GACCTTTGGCCAGGGCACCAAGCTGGAAATCAAGCGGGGCAGCACAAGCGGCAG
CGGAAAGCCTGGATCTGGCGAGGGCTCTACCAAGGGCCAGGTGCAGCTGCAGCA
GCCTGGCGCCGAAGTCGTGAAACCTGGCGCCTCCGTGAAGATGTCCTGCAAGGCC
AGCGGCTACACCTTCACCAGCTACTACATCCACTGGATCAAGCAGACCCCTGGAC
AGGGCCTGGAATGGGTGGGAGTGATCTACCCCGGCAACGACGACATCAGCTACA
ACCAGAAGTTCCAGGGCAAGGCCACCCTGACCGCCGACAAGTCTAGCACCACCG
CCTACATGCAGCTGTCCAGCCTGACCAGCGAGGACAGCGCCGTGTACTACTGCGC
CAGAGAAGTGCGGCTGCGGTACTTCGATGTGTGGGGCCAGGGAACCACCGTGAC
CGTGTCTAGCGCCCTGAGCAACAGCATCATGTACTTCAGCCACTTCGTGCCCGTG
TTTCTGCCCGCCAAGCCTACCACAACCCCTGCCCCTAGACCTCCTACCCCAGCCCC
TACAATCGCCAGCCAGCCTCTGTCTCTGAGGCCCGAGGCTTCTAGACCAGCTGCT
GGCGGAGCCGTGCACACCAGAGGCCTGGATATCTACATCTGGGCCCCACTGGCCG
GCACCTGTGGCGTGCTGCTGCTGTCTCTCGTGATCACCAAGAGAGGCCGGAAGAA
GCTGCTGTACATCTTCAAGCAGCCCTTCATGCGGCCCGTGCAGACCACCCAGGAA
GAGGACGGCTGTAGCTGCCGGTTCCCCGAGGAAGAAGAAGGGGGCTGCGAGCTG
AGAGTGAAGTTCAGCAGAAGCGCCGACGCCCCTGCCTATCAGCAGGGCCAGAAC
CAGCTGTACAACGAGCTGAACCTGGGCAGACGGGAAGAGTACGACGTGCTGGAC
AAGCGGAGAGGCAGGGACCCTGAGATGGGCGGCAAGCCCAGACGGAAGAACCC
TCAGGAAGGCCTGTATAACGAACTGCAGAAAGACAAGATGGCCGAGGCCTACTC
CGAGATCGGAATGAAGGGCGAGCGGAGAAGAGGCAAGGGCCACGATGGACTGT
ACCAGGGCCTGAGCACCGCCACCAAGGACACCTATGACGCCCTGCACATGCAGG
CCCTGCCCCCCAGATGAAATTCATCGACGTTAACTATTCTAG
CART2 nucleic acid sequence
(SEQ ID NO: 39)
GGTGTCGTGAGCGGCCGCTGAACTGGCCACCATGTGGCTGCAGTCTCTGCTGCTG
CTGGGCACCGTGGCCTGTAGCATCAGCGAGATCGTGCTGACCCAGAGCCCTGGCT
CTCTGGCTGTGTCTCCTGGCGAGCGCGTGACCATGAGCTGCAAGAGCAGCCAGAG
CGTGTTCTTCAGCAGCTCCCAGAAGAACTACCTGGCCTGGTATCAGCAGATCCCC
GGCCAGAGCCCCAGACTGCTGATCTACTGGGCCAGCACCAGAGAAAGCGGCGTG
CCCGATAGATTCACCGGCAGCGGCTCTGGCACCGACTTCACCCTGACAATCAGCA
GCGTGCAGCCCGAGGACCTGGCCATCTACTACTGCCACCAGTACCTGAGCAGCCG
GACCTTTGGCCAGGGCACCAAGCTGGAAATCAAGCGGGGCAGCACAAGCGGCAG
CGGAAAGCCTGGATCTGGCGAGGGCTCTACCAAGGGCCAGGTGCAGCTGCAGCA
GCCTGGCGCCGAAGTCGTGAAACCTGGCGCCTCCGTGAAGATGTCCTGCAAGGCC
AGCGGCTACACCTTCACCAGCTACTACATCCACTGGATCAAGCAGACCCCTGGAC
AGGGCCTGGAATGGGTGGGAGTGATCTACCCCGGCAACGACGACATCAGCTACA
ACCAGAAGTTCCAGGGCAAGGCCACCCTGACCGCCGACAAGTCTAGCACCACCG
CCTACATGCAGCTGTCCAGCCTGACCAGCGAGGACAGCGCCGTGTACTACTGCGC
CAGAGAAGTGCGGCTGCGGTACTTCGATGTGTGGGGCCAGGGAACCACCGTGAC
CGTGTCTGCCCTGAGCAACAGCATCATGTACTTCAGCCACTTCGTGCCCGTGTTTC
TGCCCGCCAAGCCTACCACAACCCCTGCCCCTAGACCTCCTACCCCAGCCCCTAC
AATCGCCAGCCAGCCTCTGTCTCTGAGGCCCGAGGCTTCTAGACCAGCTGCTGGC
GGAGCCGTGCACACCAGAGGACTGGACAAGCCCTTCTGGGTGCTGGTGGTCGTG
GGCGGAGTGCTGGCCTGTTACAGCCTGCTCGTGACAGTGGCCTTCATCATCTTTTG
GGTGCGCAGCAAGCGGTCTAGACTGCTGCACAGCGACTACATGAACATGACCCC
CAGAAGGCCAGGCCCCACCCGGAAGCACTATCAGCCTTACGCCCCTCCCAGAGA
CTTCGCCGCCTACCGGTCCAGAGTGAAGTTCAGCAGAAGCGCCGACGCCCCTGCC
TATCAGCAGGGCCAGAACCAGCTGTACAACGAGCTGAACCTGGGCAGACGGGAA
GAGTACGACGTGCTGGACAAGAGAAGAGGCCGGGACCCTGAGATGGGCGGCAA
GCCCAGACGGAAGAACCCTCAGGAAGGCCTGTATAACGAACTGCAGAAAGACAA
GATGGCCGAGGCCTACTCCGAGATCGGCATGAAGGGCGAACGGCGGAGAGGCAA
GGGACACGATGGACTGTACCAGGGCCTGAGCACCGCCACCAAGGACACCTATGA
CGCCCTGCACATGCAGGCCCTGCCCCCCAGATGAAATTCATCGACGTTAACTATT
CTAG
CART3 nucleic acid sequence
(SEQ ID NO: 40)
GGTGTCGTGAGCGGCCGCTGAACTGGCCACCATGTGGCTGCAGTCTCTGCTGCTG
CTGGGCACCGTGGCCTGTAGCATCAGCGAGATCGTGCTGACCCAGAGCCCTGGCT
CTCTGGCTGTGTCTCCTGGCGAGCGCGTGACCATGAGCTGCAAGAGCAGCCAGAG
CGTGTTCTTCAGCAGCTCCCAGAAGAACTACCTGGCCTGGTATCAGCAGATCCCC
GGCCAGAGCCCCAGACTGCTGATCTACTGGGCCAGCACCAGAGAAAGCGGCGTG
CCCGATAGATTCACCGGCAGCGGCTCTGGCACCGACTTCACCCTGACAATCAGCA
GCGTGCAGCCCGAGGACCTGGCCATCTACTACTGCCACCAGTACCTGAGCAGCCG
GACCTTTGGCCAGGGCACCAAGCTGGAAATCAAGCGGGGCAGCACAAGCGGCAG
CGGAAAGCCTGGATCTGGCGAGGGCTCTACCAAGGGCCAGGTGCAGCTGCAGCA
GCCTGGCGCCGAAGTCGTGAAACCTGGCGCCTCCGTGAAGATGTCCTGCAAGGCC
AGCGGCTACACCTTCACCAGCTACTACATCCACTGGATCAAGCAGACCCCTGGAC
AGGGCCTGGAATGGGTGGGAGTGATCTACCCCGGCAACGACGACATCAGCTACA
ACCAGAAGTTCCAGGGCAAGGCCACCCTGACCGCCGACAAGTCTAGCACCACCG
CCTACATGCAGCTGTCCAGCCTGACCAGCGAGGACAGCGCCGTGTACTACTGCGC
CAGAGAAGTGCGGCTGCGGTACTTCGATGTGTGGGGCCAGGGAACCACCGTGAC
CGTGTCTAGCGCCCTGAGCAACAGCATCATGTACTTCAGCCACTTCGTGCCCGTG
TTTCTGCCCGCCAAGCCTACCACAACCCCTGCCCCTAGACCTCCTACCCCAGCCCC
TACAATCGCCAGCCAGCCTCTGTCTCTGAGGCCCGAGGCTTCTAGACCAGCTGCT
GGCGGAGCCGTGCACACCAGAGGACTGGACAAGCCCTTCTGGGTGCTGGTGGTC
GTGGGCGGAGTGCTGGCCTGTTACAGCCTGCTCGTGACAGTGGCCTTCATCATCT
TTTGGGTGCGCAGCAAGCGGTCTAGACTGCTGCACAGCGACTACATGAACATGAC
CCCCAGAAGGCCAGGCCCCACCCGGAAGCACTATCAGCCTTACGCCCCTCCCAGA
GACTTCGCCGCCTACAGATCCAAGAGAGGCCGGAAGAAGCTGCTGTACATCTTCA
AGCAGCCCTTCATGCGGCCCGTGCAGACCACCCAGGAAGAGGACGGCTGTAGCT
GCCGGTTCCCCGAGGAAGAAGAAGGGGGCTGCGAGCTGAGAGTGAAGTTCAGCA
GAAGCGCCGACGCCCCTGCCTATCAGCAGGGCCAGAACCAGCTGTACAACGAGC
TGAACCTGGGCAGACGGGAAGAGTACGACGTGCTGGACAAGAGAAGAGGCCGG
GACCCTGAGATGGGCGGCAAGCCCAGACGGAAGAACCCTCAGGAAGGCCTGTAT
AACGAACTGCAGAAAGACAAGATGGCCGAGGCCTACTCCGAGATCGGAATGAAG
GGCGAGCGGCGGAGAGGCAAGGGACACGATGGACTGTACCAGGGCCTGAGCACC
GCCACCAAGGACACCTATGACGCCCTGCACATGCAGGCCCTGCCCCCCAGATGAA
ATTCATCGACGTTAACTATTCTAG
CART4dual nucleic acid sequence
(SEQ ID NO: 41)
GGTGTCGTGAGCGGCCGCTGAACTGGCCACCATGTGGCTGCAGTCTCTGCTGCTG
CTGGGCACCGTGGCCTGCAGCATCAGCATCCAGATGACCCAGACCACCAGCAGC
CTGAGCGCCAGCCTGGGCGATAGAGTGACCATCAGCTGCAGAGCCAGCCAGGAC
ATCAGCAAGTACCTGAACTGGTATCAGCAGAAACCCGACGGCACCGTGAAGCTG
CTGATCTACCACACCAGCAGACTGCACAGCGGCGTGCCCTCTAGATTTTCCGGCA
GCGGCTCCGGCACCGACTACAGCCTGACCATCTCCAACCTGGAACAGGAAGATAT
CGCTACCTACTTCTGTCAGCAAGGCAACACCCTGCCCTACACCTTCGGCGGAGGC
ACCAAGCTGGAAATCGGCAGCACAAGCGGCTCTGGCAAGCCTGGATCTGGCGAG
GGCTCTACCAAGGGCCTGCAGGAATCTGGCCCTGGACTGGTGGCCCCTAGCCAGA
GCCTGTCTGTGACCTGTACCGTGTCCGGCGTGTCCCTGCCTGACTATGGCGTGTCC
TGGATCAGACAGCCCCCCAGAAAGGGCCTGGAATGGCTGGGAGTGATCTGGGGC
AGCGAGACAACCTACTACAACAGCGCCCTGAAGTCCCGGCTGACCATCATCAAG
GACAACTCCAAGAGCCAGGTGTTCCTGAAGATGAACAGCCTGCAGACCGACGAC
ACCGCCATCTACTACTGCGCCAAGCACTACTACTACGGCGGCAGCTACGCCATGG
ACTACTGGGGCCAGGGCACAAGCGTGACCGTGTCTGCCCTGAGCAACAGCATCAT
GTACTTCAGCCACTTCGTGCCCGTGTTTCTGCCCGCCAAGCCTACCACAACCCCTG
CCCCTAGACCTCCTACCCCAGCCCCTACAATCGCCAGCCAGCCTCTGTCTCTGAG
GCCCGAGGCTTCTAGACCAGCTGCTGGCGGAGCCGTGCACACCAGAGGACTGGA
CAAGCCCTTCTGGGTGCTGGTGGTCGTGGGCGGAGTGCTGGCCTGTTATAGCCTG
CTCGTGACAGTGGCCTTCATCATCTTTTGGGTGCGCGTGAAGTTCAGCCGCAGCG
CCGATGCCCCTGCCTATCAGCAGGGACAGAACCAGCTGTACAACGAGCTGAACCT
GGGCAGACGGGAAGAGTACGACGTGCTGGACAAGAGAAGAGGCCGGGACCCTG
AGATGGGCGGCAAGCCCAGAAGAAAGAACCCCCAGGAAGGCCTGTATAACGAAC
TGCAGAAAGACAAGATGGCCGAGGCCTACAGCGAGATCGGCATGAAGGGCGAAC
GGCGGAGAGGCAAGGGCCACGATGGACTGTATCAGGGCCTGAGCACCGCCACCA
AGGACACCTATGACGCCCTGCACATGCAGGCTCTGCCCCCTCGCTGAAATTCATC
GACGTTAACTATTCTAG
CART5dual nucleic acid sequence
(SEQ ID NO: 42)
GGTGTCGTGAGCGGCCGCTGAACTGGCCACCATGTGGCTGCAGTCTCTGCTGCTG
CTGGGCACCGTGGCCTGTAGCATCAGCGAGATCGTGCTGACCCAGAGCCCTGGCT
CTCTGGCTGTGTCTCCTGGCGAGCGCGTGACCATGAGCTGCAAGAGCAGCCAGAG
CGTGTTCTTCAGCAGCTCCCAGAAGAACTACCTGGCCTGGTATCAGCAGATCCCC
GGCCAGAGCCCCAGACTGCTGATCTACTGGGCCAGCACCAGAGAAAGCGGCGTG
CCCGATAGATTCACCGGCAGCGGCTCTGGCACCGACTTCACCCTGACAATCAGCA
GCGTGCAGCCCGAGGACCTGGCCATCTACTACTGCCACCAGTACCTGAGCAGCCG
GACCTTTGGCCAGGGCACCAAGCTGGAAATCAAGCGGGGCAGCACAAGCGGCAG
CGGAAAGCCTGGATCTGGCGAGGGCTCTACCAAGGGCCAGGTGCAGCTGCAGCA
GCCTGGCGCCGAAGTCGTGAAACCTGGCGCCTCCGTGAAGATGTCCTGCAAGGCC
AGCGGCTACACCTTCACCAGCTACTACATCCACTGGATCAAGCAGACCCCTGGAC
AGGGCCTGGAATGGGTGGGAGTGATCTACCCCGGCAACGACGACATCAGCTACA
ACCAGAAGTTCCAGGGCAAGGCCACCCTGACCGCCGACAAGTCTAGCACCACCG
CCTACATGCAGCTGTCCAGCCTGACCAGCGAGGACAGCGCCGTGTACTACTGCGC
CAGAGAAGTGCGGCTGCGGTACTTCGATGTGTGGGGCCAGGGAACCACCGTGAC
CGTGTCTAGCGCCCTGAGCAACAGCATCATGTACTTCAGCCACTTCGTGCCCGTG
TTTCTGCCCGCCAAGCCTACCACAACCCCTGCCCCTAGACCTCCTACCCCAGCCCC
TACAATCGCCAGCCAGCCTCTGTCTCTGAGGCCCGAGGCTTCTAGACCAGCTGCT
GGCGGAGCCGTGCACACCAGAGGACTGGACAAGCCCTTCTGGGTGCTGGTGGTC
GTGGGCGGAGTGCTGGCCTGTTACAGCCTGCTCGTGACAGTGGCCTTCATCATCT
TTTGGGTGCGCAGCAAGCGGTCTAGACTGCTGCACAGCGACTACATGAACATGAC
CCCCAGAAGGCCAGGCCCCACCCGGAAGCACTATCAGCCTTACGCCCCTCCCAGA
GACTTCGCCGCCTACAGAAGCTGAAATTCATCGACGTTAACTATTCTAG
CART6 nucleic acid sequence
(SEQ ID NO: 43)
GGTGTCGTGAGCGGCCGCTGAACTGGCCACCATGTGGCTGCAGTCTCTGCTGCTG
CTGGGCACCGTGGCCTGCAGCATCAGCATCCAGATGACCCAGACCACCAGCAGC
CTGAGCGCCAGCCTGGGCGATAGAGTGACCATCAGCTGCAGAGCCAGCCAGGAC
ATCAGCAAGTACCTGAACTGGTATCAGCAGAAACCCGACGGCACCGTGAAGCTG
CTGATCTACCACACCAGCAGACTGCACAGCGGCGTGCCCTCTAGATTTTCCGGCA
GCGGCTCCGGCACCGACTACAGCCTGACCATCTCCAACCTGGAACAGGAAGATAT
CGCTACCTACTTCTGTCAGCAAGGCAACACCCTGCCCTACACCTTCGGCGGAGGC
ACCAAGCTGGAAATCGGCAGCACAAGCGGCTCTGGCAAGCCTGGATCTGGCGAG
GGCTCTACCAAGGGCCTGCAGGAATCTGGCCCTGGACTGGTGGCCCCTAGCCAGA
GCCTGTCTGTGACCTGTACCGTGTCCGGCGTGTCCCTGCCTGACTATGGCGTGTCC
TGGATCAGACAGCCCCCCAGAAAGGGCCTGGAATGGCTGGGAGTGATCTGGGGC
AGCGAGACAACCTACTACAACAGCGCCCTGAAGTCCCGGCTGACCATCATCAAG
GACAACTCCAAGAGCCAGGTGTTCCTGAAGATGAACAGCCTGCAGACCGACGAC
ACCGCCATCTACTACTGCGCCAAGCACTACTACTACGGCGGCAGCTACGCCATGG
ACTACTGGGGCCAGGGCACAAGCGTGACCGTGTCTGCCCTGAGCAACAGCATCAT
GTACTTCAGCCACTTCGTGCCCGTGTTTCTGCCCGCCAAGCCTACCACAACCCCTG
CCCCTAGACCTCCTACCCCAGCCCCTACAATCGCCAGCCAGCCTCTGTCTCTGAG
GCCCGAGGCTTCTAGACCAGCTGCTGGCGGAGCCGTGCACACCAGAGGACTGGA
CAAGCCCTTCTGGGTGCTGGTGGTCGTGGGCGGAGTGCTGGCCTGTTATAGCCTG
CTCGTGACAGTGGCCTTCATCATCTTTTGGGTGCGCAGCAAGCGGAGCCGGCTGC
TGCACTCCGACTACATGAACATGACCCCCAGACGGCCAGGCCCCACCCGGAAAC
ACTATCAGCCTTACGCCCCTCCCAGAGACTTCGCCGCCTACCGGTCCAGAGTGAA
GTTCAGCAGATCCGCCGACGCCCCTGCCTATCAGCAGGGACAGAACCAGCTGTAC
AACGAGCTGAACCTGGGCAGACGGGAAGAGTACGACGTGCTGGACAAGAGAAG
AGGCCGGGACCCTGAGATGGGCGGCAAGCCCAGAAGAAAGAACCCCCAGGAAG
GCCTGTATAACGAACTGCAGAAAGACAAGATGGCCGAGGCCTACAGCGAGATCG
GCATGAAGGGCGAACGGCGGAGAGGCAAGGGCCACGATGGACTGTATCAGGGCC
TGAGCACCGCCACCAAGGACACCTATGACGCCCTGCACATGCAGGCTCTGCCCCC
TCGCTGAAATTCATCGACGTTAACTATTCTAG
CART7 nucleic acid sequence
(SEQ ID NO: 44)
GGTGTCGTGAGCGGCCGCTGAACTGGCCACCATGTGGCTGCAGTCTCTGCTGCTG
CTGGGCACCGTGGCCTGTAGCATCAGCGAGATCGTGCTGACCCAGAGCCCTGGCT
CTCTGGCTGTGTCTCCTGGCGAGCGCGTGACCATGAGCTGCAAGAGCAGCCAGAG
CGTGTTCTTCAGCAGCTCCCAGAAGAACTACCTGGCCTGGTATCAGCAGATCCCC
GGCCAGAGCCCCAGACTGCTGATCTACTGGGCCAGCACCAGAGAAAGCGGCGTG
CCCGATAGATTCACCGGCAGCGGCTCTGGCACCGACTTCACCCTGACAATCAGCA
GCGTGCAGCCCGAGGACCTGGCCATCTACTACTGCCACCAGTACCTGAGCAGCCG
GACCTTTGGCCAGGGCACCAAGCTGGAAATCAAGCGGGGCAGCACAAGCGGCAG
CGGAAAGCCTGGATCTGGCGAGGGCTCTACCAAGGGCCAGGTGCAGCTGCAGCA
GCCTGGCGCCGAAGTCGTGAAACCTGGCGCCTCCGTGAAGATGTCCTGCAAGGCC
AGCGGCTACACCTTCACCAGCTACTACATCCACTGGATCAAGCAGACCCCTGGAC
AGGGCCTGGAATGGGTGGGAGTGATCTACCCCGGCAACGACGACATCAGCTACA
ACCAGAAGTTCCAGGGCAAGGCCACCCTGACCGCCGACAAGTCTAGCACCACCG
CCTACATGCAGCTGTCCAGCCTGACCAGCGAGGACAGCGCCGTGTACTACTGCGC
CAGAGAAGTGCGGCTGCGGTACTTCGATGTGTGGGGCCAGGGAACCACCGTGAC
CGTGTCCAGCATCGAAGTGATGTACCCCCCTCCCTACCTGGACAACGAGAAGTCC
AACGGCACCATCATCCACGTGAAGGGCAAGCACCTGTGCCCCAGCCCTCTGTTTC
CTGGCCCTAGCAAGCCCTTCTGGGTGCTGGTGGTCGTGGGCGGAGTGCTGGCCTG
TTACAGCCTGCTCGTGACAGTGGCCTTCATCATCTTTTGGGTGCGCAGCAAGCGG
TCTAGACTGCTGCACAGCGACTACATGAACATGACCCCCAGAAGGCCAGGCCCC
ACCCGGAAGCACTATCAGCCTTACGCCCCTCCCAGAGACTTCGCCGCCTACCGGT
CCAGAGTGAAGTTCAGCAGAAGCGCCGACGCCCCTGCCTATCAGCAGGGCCAGA
ACCAGCTGTACAACGAGCTGAACCTGGGCAGACGGGAAGAGTACGACGTGCTGG
ACAAGCGGAGAGGCAGGGACCCTGAGATGGGCGGCAAGCCCAGACGGAAGAAC
CCTCAGGAAGGCCTGTATAACGAACTGCAGAAAGACAAGATGGCCGAGGCCTAC
TCCGAGATCGGCATGAAGGGCGAGCGGAGAAGAGGCAAGGGCCACGATGGACT
GTACCAGGGCCTGAGCACCGCCACCAAGGACACCTATGACGCCCTGCACATGCA
GGCCCTGCCCCCCAGATGAAATTCATCGACGTTAACTATTCTAG

Any of the chimeric receptors described herein can be prepared by routine methods, such as recombinant technology. Methods for preparing the chimeric receptors herein involve generation of a nucleic acid that encodes a polypeptide comprising each of the domains of the chimeric receptors, including the antigen-binding fragment and optionally, the hinge domain, the transmembrane domain, at least one co-stimulatory signaling domain, and the cytoplasmic signaling domain. In some embodiments, a nucleic acid encoding each of the components of chimeric receptor are joined together using recombinant technology.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Alternatively, the nucleic acid encoding the chimeric receptor may be synthesized. In some embodiments, the nucleic acid is DNA. In other embodiments, the nucleic acid is RNA.

Mutation of one or more residues within one or more of the components of the chimeric receptor (e.g., the antigen-binding fragment, etc), prior to or after joining the sequences of each of the components. In some embodiments, one or more mutations in a component of the chimeric receptor may be made to modulate (increase or decrease) the affinity of the component for a target (e.g., the antigen-binding fragment for the target antigen) and/or modulate the activity of the component.

Any of the chimeric receptors described herein can be introduced into a suitable immune cell for expression via conventional technology. In some embodiments, the immune cells are T cells, such as primary T cells or T cell lines. Alternatively, the immune cells can be NK cells, such as established NK cell lines (e.g., NK-92 cells). In some embodiments, the immune cells are T cells that express CD8 (CD8⁺) or CD8 and CD4 (CD8⁺/CD4⁺). In some embodiments, the T cells are T cells of an established T cell line, for example, 293T cells or Jurkat cells.

Primary T cells may be obtained from any source, such as peripheral blood mononuclear cells (PBMCs), bone marrow, tissues such as spleen, lymph node, thymus, or tumor tissue. A source suitable for obtaining the type of immune cells desired would be evident to one of skill in the art. In some embodiments, the population of immune cells is derived from a human patient having a hematopoietic malignancy, such as from the bone marrow or from PBMCs obtained from the patient. In some embodiments, the population of immune cells is derived from a healthy donor. In some embodiments, the immune cells are obtained from the subject to whom the immune cells expressing the chimeric receptors will be subsequently administered. Immune cells that are administered to the same subject from which the cells were obtained are referred to as autologous cells, whereas immune cells that are obtained from a subject who is not the subject to whom the cells will be administered are referred to as allogeneic cells.

The type of host cells desired may be expanded within the population of cells obtained by co-incubating the cells with stimulatory molecules, for example, anti-CD3 and anti-CD28 antibodies may be used for expansion of T cells.

To construct the immune cells that express any of the chimeric receptor constructs described herein, expression vectors for stable or transient expression of the chimeric receptor construct may be constructed via conventional methods as described herein and introduced into immune host cells. For example, nucleic acids encoding the chimeric receptors may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. See section VI below. Suitable vectors and methods for producing vectors containing transgenes are well known and available in the art. Examples of the preparation of vectors for expression of chimeric receptors can be found, for example, in US2014/0106449, herein incorporated by reference in its entirety.

In some embodiments, the chimeric receptor construct or the nucleic acid encoding said chimeric receptor is a DNA molecule. In some embodiments, chimeric receptor construct or the nucleic acid encoding said chimeric receptor is a DNA vector and may be electroporated to immune cells (see, e.g., Till, et al. Blood (2012) 119(17): 3940-3950). In some embodiments, the nucleic acid encoding the chimeric receptor is an RNA molecule, which may be electroporated to immune cells.

Any of the vectors comprising a nucleic acid sequence that encodes a chimeric receptor construct described herein is also within the scope of the present disclosure. Such a vector may be delivered into host cells such as host immune cells by a suitable method. Methods of delivering vectors to immune cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Nail. Acad. Sci. USA (2013) 110(6): 2082-2087); or viral transduction. In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses. In some embodiments, the vectors for expression of the chimeric receptors are adeno-associated viruses.

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805A2, WO 1998/009271 Al, and U.S. Pat. No. 6,194,191. The viral particles are harvested from the cell culture supernatant and may be isolated and/or purified prior to contacting the viral particles with the immune cells.

The methods of preparing host cells expressing any of the chimeric receptors described herein may comprise activating and/or expanding the immune cells ex vivo. Activating a host cell means stimulating a host cell into an activate state in which the cell may be able to perform effector functions (e.g., cytotoxicity). Methods of activating a host cell will depend on the type of host cell used for expression of the chimeric receptors. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptors described herein are activated and/or expanded ex vivo prior to administration to a subject.

In some embodiments, the agents targeting a cell-surface lineage-specific antigen is an antibody-drug conjugate (ADC). As will be evident to one of ordinary skill in the art, the term “antibody-drug conjugate” can be used interchangeably with “immunotoxin” and refers to a fusion molecule comprising an antibody (or antigen-binding fragment thereof) conjugated to a toxin or drug molecule. Binding of the antibody to the corresponding antigen allows for delivery of the toxin or drug molecule to a cell that presents the antigen on the its cell surface (e.g., target cell), thereby resulting in death of the target cell.

In some embodiments, the agent is an antibody-drug conjugate. In some embodiments, the antibody-drug conjugate comprises an antigen-binding fragment and a toxin or drug that induces cytotoxicity in a target cell. In some embodiments, the antibody-drug conjugate targets a type 2 antigen. In some embodiments, the antibody-drug conjugate targets CD33 or CD19.

In some embodiments, the antigen-bind fragment of the antibody-drug conjugate has the same heavy chain CDRs as the heavy chain variable region provided by SEQ ID NO: 12 and the same light chain CDRS as the light chain variable region provided by SEQ ID NO: 13. In some embodiments, the antigen-bind fragment of the antibody-drug conjugate has the heavy chain variable region provided by SEQ ID NO: 12 and the same light chain variable region provided by SEQ ID NO: 13.

Toxins or drugs compatible for use in antibody-drug conjugate are well known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule. Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

An ADC described herein may be used as a follow-on treatment to subjects who have been undergone the combined therapy as described herein.

### Hematopoietic Cells Deficient in a Lineage-Specific Cell-Surface Antigen

The present disclosure also provides hematopoietic cells such as hematopoietic stem cells (HSCs) and/or hematopoietic progenitor cells (HPCs) that have been genetically modified to be deficient in a lineage-specific cell-surface antigen (e.g., CD33, e.g., using a gRNA described herein, e.g., wherein the gRNA comprises the nucleotide sequence of AUCCCUGGCACUCUAGAACC (SEQ ID NO: 67) or CCUCACUAGACUUGACCCAC (SEQ ID NO: 70)). In some embodiments, the cells comprise a genetic mutation at a site having a sequence of ATCCCTGGCACTCTAGAACC (SEQ ID NO: 50) or CCTCACTAGACTTGACCCAC (SEQ ID NO: 58). In some embodiments, the hematopoietic cells are HSCs, HPCs, or a combination thereof, referred to herein as “HSPCs” (“hematopoietic stem and/or progenitor cells”). In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells. HSCs are capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34⁺), which can be used for the identification and/or isolation of HSCs, and absence of cell surface markers associated with commitment to a cell lineage. Therefore, in some embodiments, the HSCs are CD34^{+.}

In some embodiments, the HSCs are obtained from a subject, such as a mammalian subject. In some embodiments, the mammalian subject is a non-human primate, a rodent (e.g., mouse or rat), a bovine, a porcine, an equine, or a domestic animal. In some embodiments, the HSCs are obtained from a human patient, such as a human patient having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

HSCs may be obtained from any suitable source using convention means known in the art. In some embodiments, HSCs are obtained from a sample from a subject, such as bone marrow sample or from a blood sample. Alternatively or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll(TM), and an intermediate fraction which contains the bone marrow cells is collected; and d) washing of the collected fraction from step (c) for recovery of re-transfusable bone marrow cells.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent. In some embodiments, the HSCs are peripheral blood HSCs.

In some embodiments, a sample is obtained from a subject and is then enriched for a desired cell type (e.g. CD34⁺/CD33⁻ cells). For example, PBMCs and/or CD34⁺ hematopoietic cells can be isolated from blood as described herein. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

Populations of HSC can be expanded prior to or after genetically engineering the HSC to become deficient in a lineage specific cell-surface antigen. The cells may be cultured under conditions that comprise an expansion medium comprising one or more cytokines, such as stem cell factor (SCF), Flt-3 ligand (F1t3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 24, 25 days or any range necessary. In some embodiments, the HSC are expanded after isolation of a desired cell population (e.g., CD34⁺/CD33⁻) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in (e.g., do not express) a cell-surface lineage-specific antigen (e.g., CD33). In some embodiments, the hematopoietic cells are genetically engineered to be deficient in the same cell-surface lineage-specific antigen that is targeted by the agent. As used herein, a hematopoietic cell is considered to be deficient in a cell-surface lineage-specific antigen if hematopoietic cell has substantially reduced expression of the cell-surface lineage-specific antigen as compared to a naturally-occurring hematopoietic cell of the same type as the genetically engineered hematopoietic cell (e.g., is characterized by the presence of the same cell surface markers, such as CD34). In some embodiments, the hematopoietic cell has no detectable expression of the cell-surface lineage-specific antigen (e.g., does not express the cell-surface lineage-specific antigen). The expression level of a cell-surface lineage-specific antigen can be assessed by any means known in the art. For example, the expression level of a cell-surface lineage-specific antigen can be assessed by detecting the antigen with an antigen-specific antibody (e.g., flow cytometry methods, Western blotting).

In some embodiments, the expression of the cell-surface lineage-specific antigen on the genetically engineered hematopoietic cell is compared to the expression of the cell-surface lineage-specific antigen on a naturally occurring hematopoietic cell (e.g., a wild-type counterpart). In some embodiments, the genetic engineering results in a reduction in the expression level of the cell-surface lineage-specific antigen by at least about 50%, 60%, 70%, 80%, 90%, 91%, 92%, 93%, 94%, 95%, 96%, 97%, 98%, or 99% as compared to the expression of the cell-surface lineage-specific antigen on a naturally occurring hematopoietic cell. That is, in some embodiments, the genetically engineered hematopoietic cell expresses less than about 20%, 19%, 18%, 17%, 16%, 15%, 14%, 13%, 12%, 11%, 10%, 9%, 8%, 7%, 6%, 5%, 4%, 3%, 2%, or 1% of the cell-surface lineage-specific antigen (e.g., CD33) as compared to a naturally occurring hematopoietic cell (e.g., a wild-type counterpart).

In some embodiments, the genetic engineering results in a reduction in the expression level of a wild-type cell-surface lineage-specific antigen (e.g., CD33) by at least about 50%, 60%, 70%, 80%, 90%, 91%, 92%, 93%, 94%, 95%, 96%, 97%, 98%, or 99% as compared to the expression of the level of the wild-type cell-surface lineage-specific antigen on a naturally occurring hematopoietic cell. That is, in some embodiments, the genetically engineered hematopoietic cell expresses less than about 20%, 19%, 18%, 17%, 16%, 15%, 14%, 13%, 12%, 11%, 10%, 9%, 8%, 7%, 6%, 5%, 4%, 3%, 2%, or 1% of a wild-type cell-surface lineage-specific antigen (e.g., CD33) as compared to a naturally occurring hematopoietic cell (e.g., a wild-type counterpart).

In some embodiments, the hematopoietic cell is deficient in the whole endogenous gene encoding the cell-surface lineage-specific antigen. In some embodiments, the whole endogenous gene encoding the cell-surface lineage-specific antigen has been deleted. In some embodiments, the hematopoietic cell comprises a portion of endogenous gene encoding the cell-surface lineage-specific antigen. In some embodiments, the hematopoietic cell expressing a portion (e.g. a truncated protein) of the cell-surface lineage-specific antigen. In other embodiments, a portion of the endogenous gene encoding the cell-surface lineage-specific antigen has been deleted. In some embodiments, at least 10%, 20%, 30%, 40%, 50%, 60%, 70% or more of the gene encoding the cell-surface lineage-specific antigen has been deleted.

As will be appreciated by one of ordinary skill in the art, a portion of the nucleotide sequence encoding the cell-surface lineage-specific antigen may be deleted or one or more non-coding sequences, such that the hematopoietic cell is deficient in the antigen (e.g., has substantially reduced expression of the antigen).

In some embodiments, the cell-surface lineage-specific antigen is CD33. The predicted structure of CD33 includes two immunoglobulin domains, an IgV domain and an IgC2 domain. In some embodiments, a portion of the immunoglobulin C domain of CD33 is deleted.

Any of the genetically engineering hematopoietic cells, such as HSCs, that are deficient in a cell-surface lineage-specific antigen can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to knock out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems.

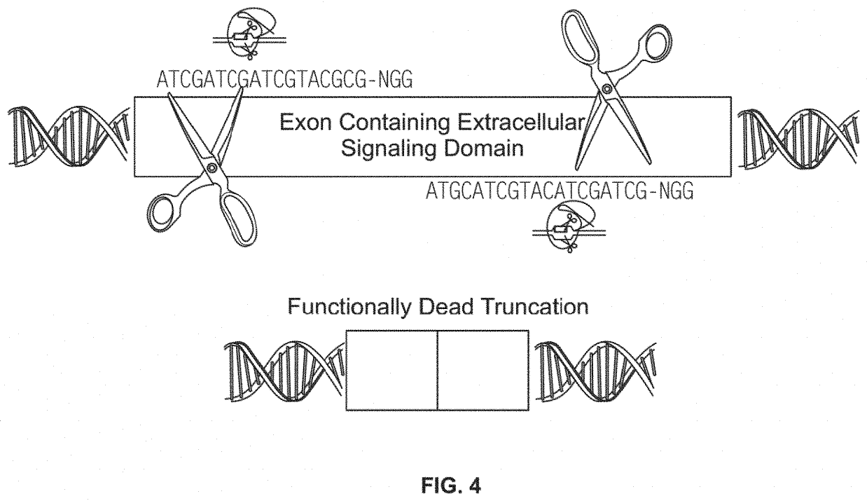

In one aspect of the present disclosure, the replacement of the tumor cells by a modified population of normal cells is performed using normal cells in which a lineage-specific antigen is modified. Such modification may include the depletion or inhibition of any lineage specific antigen using a CRISPR-Cas9 system, where the Clustered Regularly Interspaced Short Palindromic Repeats (CRISPR)-Cas9 system is an engineered, non-naturally occurring CRISPR-Cas9 system (FIG. 4).

CRISPR-Cas system has been successfully utilized to edit the genomes of various organisms, including, but not limited to bacteria, humans, fruit flies, zebra fish and plants. See, e.g., Jiang et al., Nature Biotechnology (2013) 31(3):233; Qi et al, Cell (2013) 5:1173; DiCarlo et al., Nucleic Acids Res. (2013) 7:4336; Hwang et al., Nat. Biotechnol (2013), 3:227); Gratz et al., Genetics (2013) 194:1029; Cong et al., Science (2013) 6121:819; Mali et al., Science (2013) 6121:823; Cho et al. Nat. Biotechnol (2013) 3: 230; and Jiang et al., Nucleic Acids Research (2013) 41(20):e188.

The present disclosure utilizes the CRISPR/Cas9 system that hybridizes with a target sequence in a lineage specific antigen polynucleotide, where the CRISPR/Cas9 system comprises a Cas9 nuclease and an engineered crRNA/tracrRNA (or single guide RNA). CRISPR/Cas9 complex can bind to the lineage specific antigen polynucleotide and allow the cleavage of the antigen polynucleotide, thereby modifying the polynucleotide.

The CRISPR/Cas system of the present disclosure may bind to and/or cleave the region of interest within a cell-surface lineage-specific antigen in a coding or non-coding region, within or adjacent to the gene, such as, for example, a leader sequence, trailer sequence or intron, or within a non-transcribed region, either upstream or downstream of the coding region. The guide RNAs (gRNAs) used in the present disclosure may be designed such that the gRNA directs binding of the Cas9-gRNA complexes to a pre-determined cleavage sites (target site) in a genome. The cleavage sites may be chosen so as to release a fragment that contains a region of unknown sequence, or a region containing a SNP, nucleotide insertion, nucleotide deletion, rearrangement, etc.

Cleavage of a gene region may comprise cleaving one or two strands at the location of the target sequence by the Cas enzyme. In one embodiment, such, cleavage can result in decreased transcription of a target gene. In another embodiment, the cleavage can further comprise repairing the cleaved target polynucleotide by homologous recombination with an exogenous template polynucleotide, wherein the repair results in an insertion, deletion, or substitution of one or more nucleotides of the target polynucleotide.

The terms “gRNA,” “guide RNA” and “CRISPR guide sequence” may be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length.

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

In some embodiments, the gRNA is modified, e.g., is chemically modified. The modified gRNA comprises at least one nucleotide having a modification to the chemical structure of at least one of the following: nucleobase, sugar, and phosphodiester linkage or backbone portion (e.g., nucleotide phosphates). Exemplary gRNA modifications will be evident to one of skill in the art and can be found, for example, in Lee et al., Synthetically modified guide RNA and donor DNA are a versatile platform for CRISPR-Cas9 engineering. Elife. 2017 May 2;6. pii: e25312. doi:10.7554/eLife.25312 and U.S. Publication 2016/0289675. Additional suitable modifications include phosphorothioate backbone modification, 2′-O-Me-modified sugar, 2′F-modified sugar, replacement of the ribose sugar with the bicyclic nucleotide-cEt, 3′ thioPACE (MSP), or any combination thereof. Suitable gRNA modifications are described, e.g., in Randar et al. PNAS December 22, 2015 112 (51) E7110-E7117 and Hendel et al., Nat Biotechnol. 2015 Sep; 33(9): 985-989, each of which is incorporated herein by reference in its entirety.

In some embodiments, a gRNA described herein is chemically modified. For example, the gRNA may comprise one or more 2′-O modified nucleotide, e.g., 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′ phosphorous-modified nucleotide, e.g., a 2′-O-methyl 3′ phosphorothioate nucleotide. In some embodiments, the gRNA comprises a 2′-O-modified and 3′ phosphorous-modified, e.g., 2′-O-methyl 3′ phosphorothioate nucleotide at the 5′ end of the gRNA. In some embodiments, the gRNA comprises a 2′-O-modified and 3′ phosphorous-modified, e.g., 2′-O-methyl 3′ phosphorothioate nucleotide at the 3′ end of the gRNA. In some embodiments, the gRNA comprises a 2′-O-modified and 3′ phosphorous-modified, e.g., 2′-O-methyl 3′ phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′ phosphorous-modified, e.g. 2′-O-methyl 3′ phosphorothioate-modified at the nucleotide at the 5′ end of the gRNA, the second nucleotide from the 5′ end of the gRNA, and the third nucleotide from the 5′ end of the gRNA.

In some embodiments, the gRNA is 2′-O-modified and 3′ phosphorous-modified, e.g. 2′-O-methyl 3′ phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′ phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′ phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′ phosphorous-modified, e.g. 2′-O-methyl 3′ phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′ thioPACE nucleotide. In some embodiments, the gRNA comprises a 2′-O-modified and 3′ phosphorous-modified, e.g., 2′-O-methyl 3′ thioPACE nucleotide at the 5′ end of the gRNA. In some embodiments, the gRNA comprises a 2′-O-modified and 3′ phosphorous-modified, e.g., 2′-O-methyl 3′ thioPACE nucleotide at the 3′ end of the gRNA. In some embodiments, the gRNA comprises a 2′-O-modified and 3′ phosphorous-modified, e.g., 2′-O-methyl 3′ thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′ phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′ phosphorous-modified, e.g. 2′-O-methyl 3′ thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′ thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′ phosphorous-modified, e.g. 2′-O-methyl 3′ thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′ phosphorous-modified, e.g. 2′-O-methyl 3′ thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments , the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments , the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Chemical modifications of gRNAs are described, for example, in Hendel, A. et al., Nature Biotech., 2015, Vol 33, No. 9, which is herein incorporated by reference in its entirety.

As used herein, a “scaffold sequence,” also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences will be evident to one of skill in the art and can be found, for example, in Jinek, et al. Science (2012) 337(6096):816-821, Ran, et al. Nature Protocols (2013) 8:2281-2308, PCT Application No. WO2014/093694, and PCT Application No. WO2013/176772.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (Upadhyay, et al. Genes Genome Genetics (2013) 3(12):2233-2238). In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The target nucleic acid is flanked on the 3′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG. For Cas9 endonucleases derived from Staphylococcus aureus, the PAM sequence is NNGRRT. For Cas9 endonucleases that are derived from Neisseria meningitidis, the PAM sequence is NNNNGATT. For Cas9 endonucleases derived from Streptococcus therrnophilus, the PAM sequence is NNAGAA. For Cas9 endonuclease derived from Treponema denticola, the PAM sequence is NAAAAC. For a Cpf1 nuclease, the PAM sequence is TTN.

In some embodiments, genetically engineering a cell also comprises introducing one or more (e.g., 1, 2, 3 or more) Cas endonuclease into the cell. In some embodiments, the Cas endonuclease and the nucleic acid encoding the gRNA are provided on the same nucleic acid (e.g., a vector). In some embodiments, the Cas endonuclease and the nucleic acid encoding the gRNA are provided on different nucleic acids (e.g., different vectors). Alternatively or in addition, the Cas endonuclease may be provided or introduced into the cell in protein form.

In some embodiments, the Cas endonuclease is a Cas9 enzyme or variant thereof. In some embodiments, the Cas9 endonuclease is derived from Streptococcus pyogenes (SpCas9), Staphylococcus aureus (SaCas9), Neisseria meningitidis (NmCas9), Streptococcus therrnophilus, Campylobacter jejuni (CjCas9), or Treponema denticola. In some embodiments, the nucleotide sequence encoding the Cas endonuclease may be codon optimized for expression in a host cell. In some embodiments, the endonuclease is a Cas9 homolog or ortholog. In some embodiments, the nucleotide sequence encoding the Cas9 endonuclease is further modified to alter the activity of the protein. In some embodiments, the Cas9 endonuclease has been modified to inactivate one of the catalytic residues of the endonuclease, referred to as a “nickase” or “Cas9n”. Cas9 nickase endonucleases cleave one DNA strand of the target nucleic acid. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 endonuclease is a catalytically inactive Cas9. For example, dCas9 contains mutations of catalytically active residues (D10 and H840) and does not have nuclease activity. Alternatively or in addition, the Cas9 endonuclease may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g., CRISPR interference (CRISPRi)). In some embodiments, dCas9 is fused to an activator domain, such as VP64 or VPR. In some embodiments, such dCas9 fusion proteins are used with the constructs described herein for gene activation (e.g., CRISPR activation (CRISPRa)). In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation. In some embodiments, dCas9 or Cas9 is fused to a Fok1 nuclease domain. In some embodiments, Cas9 or dCas9 fused to a Fok1 nuclease domain is used for genome editing. In some embodiments, Cas9 or dCas9 is fused to a fluorescent protein (e.g., GFP, RFP, mCherry, etc.). In some embodiments, Cas9/dCas9 proteins fused to fluorescent proteins are used for labeling and/or visualization of genomic loci or identifying cells expressing the Cas endonuclease.

In some embodiments, the Cas endonuclease is modified to enhance specificity of the enzyme (e.g., reduce off-target effects, maintain robust on-target cleavage). In some embodiments, the Cas endonuclease is an enhanced specificity Cas9 variant (e.g., eSPCas9). See, e.g., Slaymaker et al. Science (2016) 351 (6268): 84-88. In some embodiments, the Cas endonuclease is a high fidelity Cas9 variant (e.g., SpCas9-HF1). See, e.g., Kleinstiver et al. Nature (2016) 529: 490-495.

Cas enzymes, such as Cas endonucleases, are known in the art and may be obtained from various sources and/or engineered/modified to modulate one or more activities or specificities of the enzymes. In some embodiments, the Cas enzyme has been engineered/modified to recognize one or more PAM sequence. In some embodiments, the Cas enzyme has been engineered/modified to recognize one or more PAM sequence that is different than the PAM sequence the Cas enzyme recognizes without engineering/modification. In some embodiments, the Cas enzyme has been engineered/modified to reduce off-target activity of the enzyme.

In some embodiments, the nucleotide sequence encoding the Cas endonuclease is modified further to alter the specificity of the endonuclease activity (e.g., reduce off-target cleavage, decrease the Cas endonuclease activity or lifetime in cells, increase homology-directed recombination and reduce non-homologous end joining). See, e.g., Komor et al. Cell (2017) 168: 20-36. In some embodiments, the nucleotide sequence encoding the Cas endonuclease is modified to alter the PAM recognition of the endonuclease. For example, the Cas endonuclease SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas endonuclease is considered “relaxed” if the Cas endonuclease recognizes more potential PAM sequences as compared to the Cas endonuclease that has not been modified. For example, the Cas endonuclease SaCas9 recognizes PAM sequence NNGRRT, whereas a relaxed variant of the SaCas9 comprising one or more modifications of the endonuclease (e.g., KKH SaCas9) may recognize the PAM sequence NNNRRT. In one example, the Cas endonuclease FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas endonuclease is a Cpf 1 endonuclease comprising substitution mutations S542R and K607R and recognize the PAM sequence TYCV. In one example, the Cas endonuclease is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas endonucleases are used. In some embodiments, at least one of the Cas endonucleases is a Cas9 enzyme. In some embodiments, at least one of the Cas endonucleases is a Cpf1 enzyme. In some embodiments, at least one of the Cas9 endonucleases is derived from Streptococcus pyogenes. In some embodiments, at least one of the Cas9 endonuclease is derived from Streptococcus pyogenes and at least one Cas9 endonuclease is derived from an organism that is not Streptococcus pyogenes. In some embodiments, the endonuclease is a base editor. Base editor endonuclease generally comprises a catalytically inactive Cas endonuclease fused to a function domain. See, e.g., Eid et al. Biochem. J. (2018) 475(11): 1955-1964; Rees et al. Nature Reviews Genetics (2018) 19:770-788. In some embodiments, the catalytically inactive Cas endonuclease is dCas9. In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas endonuclease has reduced activity and is nCas9. In some embodiments, the endonuclease comprises a nCas9 fused to one or more uracil glycosylase inhibitor (UGI) domains. In some embodiments, the endonuclease comprises a nCas9 fused to an adenine base editor (ABE), for example an ABE evolved from the RNA adenine deaminase TadA. In some embodiments, the endonuclease comprises a nCas9 fused to cytodine deaminase enzyme (e.g., APOBEC deaminase, pmCDA1, activation-induced cytidine deaminase (AID)).

Examples of base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas endonucleases described herein may be fused to a Gam domain (bacteriophage Mu protein) to protect the Cas endonuclease from degradation and exonuclease activity. See, e.g., Eid et al. Biochem. J. (2018) 475(11): 1955-1964.

In some embodiments, the Cas endonuclease belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017). In some embodiments, the Cas endonuclease is a type V-A Cas endonuclease, such as a Cpf1 nuclease. In some embodiments, the Cas endonuclease is a type V-B Cas endonuclease, such as a C2c1 endonuclease. See, e.g., Shmakov et al. Mol Cell (2015) 60: 385-397. In some embodiments, the Cas endonuclease is Mad7.

Alternatively or in addition, the Cas endonuclease is a Cpf1 nuclease or a variant thereof. As will be appreciated by one of skill in the art, the Cas endonuclease Cpf1 nuclease may also be referred to as Cas12a. See, e.g., Strohkendl et al. Mol. Cell (2018) 71: 1-9. In some embodiments, the host cell expresses a Cpf1 nuclease derived from Provetella spp. or Francisella spp., Acidaminococcus sp. (AsCpf1 ), Lachnospiraceae bacterium (LpCpf1 ), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas endonuclease is dCas9. In some embodiments, the endonuclease comprises a dCas12a fused to one or more uracil glycosylase inhibitor (UGI) domains. In some embodiments, the endonuclease comprises a dCas12a fused to an adenine base editor (ABE), for example an ABE evolved from the RNA adenine deaminase TadA. In some embodiments, the endonuclease comprises a dCasl2a fused to cytodine deaminase enzyme (e.g., APOBEC deaminase, pmCDA1, activation-induced cytidine deaminase (AID)).

Alternatively or in addition, the Cas endonuclease may be a Cas14 endonuclease or variant thereof. In contrast to Cas9 endonucleases, Cas14 endonucleases are derived from archaea and tend to be smaller in size (e.g., 400-700 amino acids). Additionally Cas14 endonucleases do not require a PAM sequence. See, e.g., Harrington et al. Science (2018).

Any of the Cas endonucleases described herein may be modulated to regulate levels of expression and/or activity of the Cas endonuclease at a desired time. For example, it may be advantageous to increase levels of expression and/or activity of the Cas endonuclease during particular phase(s) of the cell cycle. It has been demonstrated that levels of homology-directed repair are reduced during the G1 phase of the cell cycle, therefore increasing levels of expression and/or activity of the Cas endonuclease during the S phase, G2 phase, and/or M phase may increase homology-directed repair following the Cas endonuclease editing. In some embodiments, levels of expression and/or activity of the Cas endonuclease are increased during the S phase, G2 phase, and/or M phase of the cell cycle. In one example, the Cas endonuclease fused to a the N-terminal region of human Geminin. See, e.g., Gutschner et al. Cell Rep. (2016) 14(6): 1555-1566. In some embodiments, levels of expression and/or activity of the Cas endonuclease are reduced during the G1 phase. In one example, the Cas endonuclease is modified such that it has reduced activity during the G1 phase. See, e.g., Lomova et al. Stem Cells (2018).

Alternatively or in addition, any of the Cas endonucleases described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas endonucleases fused to an epigenetic modifier may be referred to as “epieffectors” and may allow for temporal and/or transient endonuclease activity. In some embodiments, the Cas endonuclease is a dCas9 fused to a chromatin-modifying enzyme.

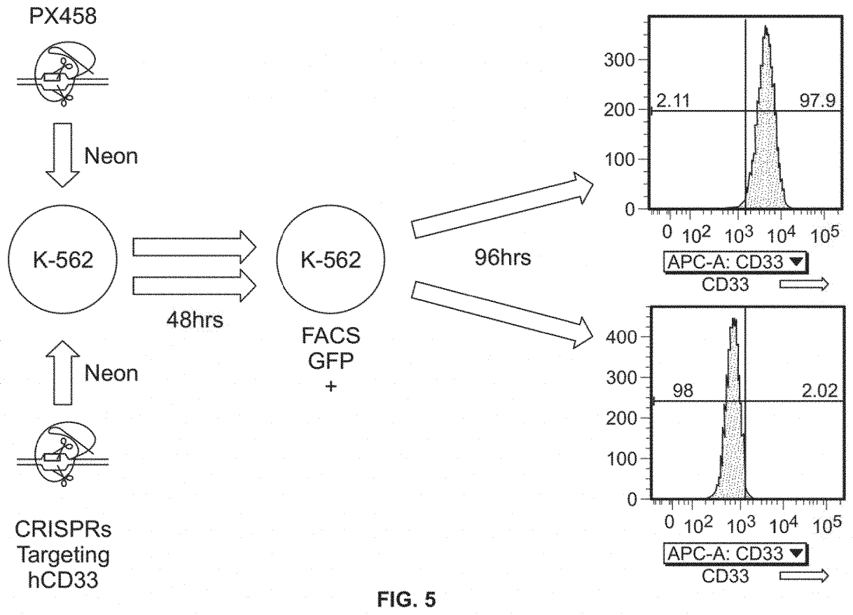

In some embodiments, the present disclosure provides compositions and methods for inhibiting a cell-surface lineage-specific antigen in hematopoietic cells using a CRISPR/Cas9 system, wherein guide RNA sequence hybridizes to the nucleotide sequence encoding the cell-surface lineage-specific antigen. In some embodiments, the cell-surface lineage-specific antigen is CD33 and the gRNA hybridizes to a portion of the nucleotide sequence that encodes the CD33 (FIG. 5). Examples of gRNAs that target CD33 are provided in Table 4, although additional gRNAs may be developed that hybridize to CD33 and can be used in the methods described herein. In some embodiments, the gRNA comprises SEQ ID NO: 50 or SEQ ID NO: 51.

Table 4 provides exemplary guide RNA sequences that hybridize or are predicted to hybridize to a portion of CD33. While both the RNA and DNA sequences of the exemplary guide RNA sequences are provided, the skilled artisan will appreciate that, except where otherwise noted, polynucleotide sequences set forth in the instant application will recite “T”'s in a representative DNA sequence but where the sequence represents RNA, the “T”'s would be substituted for “U”'s.

TABLE 4
Guide RNA sequences targeting CD33 (both DNA and RNA sequences provided)

| Name | Guide sequence | Guide position | Score |
|---|---|---|---|
| hCD33-IgC1 | ACCTGTCAGGTGAAGTTCGC TGG | Chr19:+51729270 | 87 |
| referred to herein | (SEQ ID NO: 11) | | |
| as ″Crispr 1″ | ACCUGUCAGGUGAAGUUCGC | | |
| | UGG | | |
| | (SEQ ID NO: 60) | | |
| hCD33-IgC3 | TGGCCGGGTTCTAGAGTGCC AGG | Chr19:-51729087 | 82 |
| referred to herein | (SEQ ID NO: 28) | | |
| as ″Crispr 3″ | UGGCCGGGUUCUAGAGUGCC | | |
| | AGG | | |
| | (SEQ ID NO: 61) | | |
| hCD33-IgC5 | GGCCGGGTTCTAGAGTGCCA GGG | Chr19:-51729086 | 81 |
| referred to herein | (SEQ ID NO: 29) | | |
| as ″Crispr 5″ | GGCCGGGUUCUAGAGUGCCA | | |
| | GGG | | |
| | (SEQ ID NO: 62) | | |
| hCD33 gRNA | CACCGAGGAGTGAGTAGTCC TGG | | |
| | (SEQ ID NO: 30) | | |
| | CACCGAGGAGUGAGUAGUCC | | |
| | UGG (SEQ ID NO: 63) | | |
| hCD33 gRNA | TCCAGCGAACTTCACCTGAC AGG | | |
| | (SEQ ID NO: 31) | | |
| | UCCAGCGAACUUCACCUGAC | | |
| | AGG (SEQ ID NO: 64) | | |
| hCD33 gRNA | CCTCACTAGACTTGACCCAC AGG | Chr19, (+) strand, | |
| | (SEQ ID NO: 48) | start 51225794, end | |
| | CCUCACUAGACUUGACCCAC | 51225813 | |
| | AGG | | |
| | (SEQ ID NO: 65) | | |
| hCD33 gRNA | ATCCCTGGCACTCTAGAACC CGG | Chr19, (+) strand, | |
| | (SEQ ID NO: 49) | start 51225829, end | |
| | AUCCCUGGCACUCUAGAACC | 51225848 | |
| | CGG | | |
| | (SEQ ID NO: 66) | | |
| hCD33 gRNA | ATCCCTGGCACTCTAGAACC | | |
| | (SEQ ID NO: 50) | | |
| | AUCCCUGGCACUCUAGAACC | | |
| | (SEQ ID NO: 67) | | |
| hCD33 gRNA | GGCCGGGTTCTAGAGTGCCA | | |
| | (SEQ ID NO: 51) | | |
| | GGCCGGGUUCUAGAGUGCCA | | |
| | (SEQ ID NO: 68) | | |
| hCD33 gRNA | CCTCACTAGACTTGACCCAC | | |
| | (SEQ ID NO:58) | | |
| | CCUCACUAGACUUGACCCAC | | |
| | (SEQ ID NO: 70) | | |

In some instances, the gRNA for use in the present disclosure may comprise a spacer sequence at least 90% (e.g., at least 93%, 95%, 96%, 97%, 98%, or 99%) identical to any of the exemplary guide RNA sequences in Table 4 above, for example, SEQ ID NO:67, SEQ ID NO:68, or SEQ ID NO:70.

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, it may be desired to further genetically engineer the HSC, particularly allogeneic HSCs, to reduce the graft-versus-host effects. For example, the standard therapy for relapsed AML is hematopoietic stem cell transplantation (HSCT). However, at least one of the limiting factors for successful HSCT is graft-versus-host disease (GVHD), which expression of the cell surface molecule CD45 has been implicated. See, e.g., Van Besie, Hematology Am. Soc. Hematol Educ Program (2013)56; Mawad Curr. Hematol. Malig. Rep. (2013) 8(2):132. CD45RA and CD45RO are isoforms of CD45 (found on all hematopoietic cells except erythrocytes). In T lymphocytes, CD45RA is expressed on naive cells, while CD45RO is expressed on memory cells. CD45RA T cells have a high potential for reactivity against recipient-specific antigens following HSCT, resulting in GVHD. Thus, there remains a need for efficient and safe AML treatment that would also reduce the possibility of transplant rejection or GVHD. CD45 is a type 1 lineage antigen, since CD45 bearing cells are required for survival but the antigen may be deleted from stem cells using CRISPR.

Taking into account the complications arising due to the development of GvHD following HSCT, the present disclosure also provides compositions and methods for targeting CD45RA. Such compositions and methods are meant to prevent and/or reduce the incidence or extent of GvHD.

Thus, in the case of GVHD, the treatment of the patient can involve the following steps: (1) administering a therapeutically effective amount of a T cell to the patient, where the T cell comprises a nucleic acid sequence encoding a chimeric antigen receptor (CAR) targeting CD45RA lineage specific antigen; and (2) infusing the patient with hematopoietic stem cells, where the hematopoietic cells have reduced expression of CD45RA lineage specific antigen.

Additionally, the present disclosure provides compositions and methods for the combined inhibition of both CD33 and CD45RA lineage specific antigens. Such treatment regimen can involve the following steps: (1) administering a therapeutically effective amount of a T cell to the patient, where the T cell comprises a nucleic acid sequence encoding a chimeric antigen receptor (CAR) targeting both CD33 and CD45RA lineage specific antigens; and (2) infusing or reinfusing the patient with hematopoietic stem cells, either autologous or allogeneic, where the hematopoietic cells have reduced expression of both the CD33 and CD45RA lineage specific antigens.

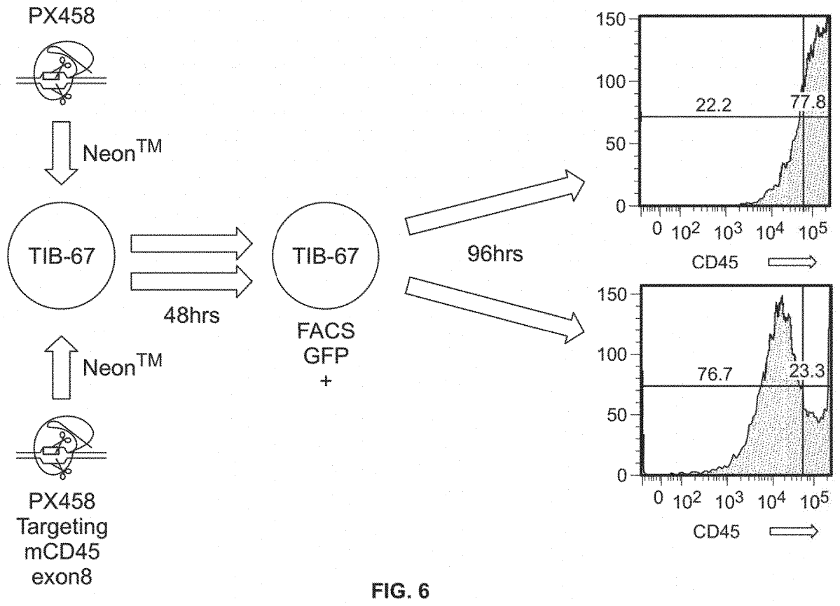

In some embodiments, the cell-surface lineage-specific antigen CD45RA is also deleted or inhibited in the hematopoietic cells using a CRISPR/Cas9 system. In some embodiments, the gRNA sequence hybridizes to a portion of the nucleotide sequence encoding CD45RA (FIG. 6). Examples of gRNAs that target CD45RA are provided in Table 5, although additional gRNAs may be developed that hybridize to CD45RA and can be used in the methods described herein.

Table 5 provides exemplary guide RNA sequences that hybridize or are predicted to hybridize to exon 4 or exon 5 of human CD45.

TABLE 5
Guide RNA sequences targeting CD45

| hCD45 Target | Guide RNA |
|---|---|
| Exon 4 | CCAAAGAGTCCGGGGATACT TGG |
| | (SEQ ID NO: 9) |
| | CCAAGTATCCCCGGACTCTT TGG |
| | (SEQ ID NO: 32) |
| | AGCATTATCCAAAGAGTCCG GGG |
| | (SEQ ID NO: 33) |
| | ACTTGGGTGGAAGTATTGTC TGG |
| | (SEQ ID NO: 34) |
| Exon 5 | GTTGAGTTTTGCATTGGCGG CGG |
| | (SEQ ID NO: 10) |
| | GTCTGCGAGTCTGCGTGCGT GGG |
| | (SEQ ID NO: 35) |
| | CGTCTGCGAGTCTGCGTGCG TGG |
| | (SEQ ID NO: 36) |
| | GCGAGTCTGCGTGCGTGGGA AGG |
| | (SEQ ID NO: 37) |

Also provided herein are methods of producing a cell that is deficient in a cell-surface lineage-specific antigen involving providing a cell and introducing into the cell components of a CRISPR Cas system for genome editing. In some embodiments, a nucleic acid that comprises a CRISPR-Cas guide RNA (gRNA) that hybridizes or is predicted to hybridize to a portion of the nucleotide sequence that encodes the lineage-specific cell-surface antigen is introduced into the cell. In some embodiments, the gRNA is introduced into the cell on a vector. In some embodiments, a Cas endonuclease is introduced into the cell. In some embodiments, the Cas endonuclease is introduced into the cell as a nucleic acid encoding a Cas endonuclease. In some embodiments, the gRNA and a nucleotide sequence encoding a Cas endonuclease are introduced into the cell on the same nucleic acid (e.g., the same vector). IN some embodiments, the Cas endonuclease is introduced into the cell in the form of a protein. In some embodiments, the Cas endonuclease and the gRNA are pre-formed in vitro and are introduced to the cell in as a complex.

The present disclosure further provides engineered, non-naturally occurring vectors and vector systems, which can encode one or more components of a CRISPR/Cas9 complex, wherein the vector comprises a polynucleotide encoding (i) a (CRISPR)-Cas system guide RNA that hybridizes to the lineage specific antigen sequence and (ii) a Cas9 endonuclease.

Vectors of the present disclosure can drive the expression of one or more sequences in mammalian cells using a mammalian expression vector. Examples of mammalian expression vectors include pCDM8 (Seed, Nature (1987) 329: 840) and pMT2PC (Kaufman, et al., EMBO J. (1987) 6: 187). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989.

The vectors of the present disclosure are capable of directing expression of the nucleic acid preferentially in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Conventional viral and non-viral based gene transfer methods can be used to introduce nucleic acids encoding CRISPR/Cas9 in mammalian cells or target tissues. Such methods can be used to administer nucleic acids encoding components of a CRISPR-Cas system to cells in culture, or in a host organism.

Non-viral vector delivery systems include DNA plasmids, RNA (e.g., a transcript of a vector described herein), naked nucleic acid, and nucleic acid complexed with a delivery vehicle. In one embodiment, the non-viral vector delivery system used is a pre-formed ribonucleoprotein complex (e.g., a complex comprising a Cas9 protein in complex with the targeting gRNA). The pre-formed ribonucleoprotein complex may then be introduced into the cell via electroporation, biolistic bombardment, or other physical methods of delivery. In one embodiment, electroporation is used to introduce the pre-formed ribonucleoprotein complex into the cell.

Viral vector delivery systems include DNA and RNA viruses, which have either episomal or integrated genomes after delivery to the cell. Viral vectors can be administered directly to patients (in vivo) or they can be used to manipulate cells in vitro or ex vivo, where the modified cells may be administered to patients. In one embodiment, the present disclosure utilizes viral based systems including, but not limited to retroviral, lentivirus, adenoviral, adeno-associated and herpes simplex virus vectors for gene transfer. Furthermore, the present disclosure provides vectors capable of integration in the host genome, such as retrovirus or lentivirus. Preferably, the vector used for the expression of a CRISPR-Cas system of the present disclosure is a lentiviral vector.

In one embodiment, the disclosure provides for introducing one or more vectors encoding CRISPR-Cas into eukaryotic cell. The cell can be a cancer cell. Alternatively, the cell is a hematopoietic cell, such as a hematopoietic stem cell. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs). In a preferred embodiment, the disclosure provides introducing CRISPR-Cas9 into a hematopoietic stem cell.

The vectors of the present disclosure are delivered to the eukaryotic cell in a subject. Modification of the eukaryotic cells via CRISPR/Cas9 system can takes place in a cell culture, where the method comprises isolating the eukaryotic cell from a subject prior to the modification. In some embodiments, the method further comprises returning said eukaryotic cell and/or cells derived therefrom to the subject.

### Combined Therapy

As described herein, agents comprising an antigen-binding fragment that binds to a cell-surface lineage-specific antigen (e.g., CD33) may be administered to a subject in combination with hematopoietic cells that are deficient for the cell-surface lineage-specific antigen, e.g., hematopoietic stem or progenitor cells produced using a gRNA described herein, e.g., wherein the gRNA comprises the nucleotide sequence of AUCCCUGGCACUCUAGAACC (SEQ ID NO: 67) or CCUCACUAGACUUGACCCAC (SEQ ID NO: 70)). In some embodiments, the cells comprise a genetic mutation at a site having a sequence of ATCCCTGGCACTCTAGAACC (SEQ ID NO: 50) or CCTCACTAGACTTGACCCAC (SEQ ID NO: 58). As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human patient having a hematopoietic malignancy.

In some embodiments, the agents and/or the hematopoietic cells may be mixed with a pharmaceutically acceptable carrier to form a pharmaceutical composition, which is also within the scope of the present disclosure.

To perform the methods described herein, an effective amount of the agent comprising an antigen-binding fragment that binds to a cell-surface lineage-specific antigen and an effective amount of hematopoietic cells can be co-administered to a subject in need of the treatment. As used herein the term “effective amount” may be used interchangeably with the term “therapeutically effective amount” and refers to that quantity of an agent, cell population, or pharmaceutical composition (e.g., a composition comprising agents and/or hematopoietic cells) that is sufficient to result in a desired activity upon administration to a subject in need thereof. Within the context of the present disclosure, the term “effective amount” refers to that quantity of a compound, cell population, or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination may or may not include amounts of each ingredient that would have been effective if administered individually.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy.

As described herein, the hematopoietic cells and/or immune cells expressing chimeric receptors may be autologous to the subject, i.e., the cells are obtained from the subject in need of the treatment, genetically engineered to be deficient for expression of the cell-surface lineage-specific antigen or for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered to be deficient for expression of the cell-surface lineage-specific antigen or for expression of the chimeric receptor constructs, and administered to a second subject that is different from the first subject but of the same species. For example, allogeneic immune cells may be derived from a human donor and administered to a human recipient who is different from the donor.

In some embodiments, the immune cells and/or hematopoietic cells are allogeneic cells and have been further genetically engineered to reduced graft-versus-host disease. For example, as described herein, the hematopoietic stem cells may be genetically engineered (e.g., using genome editing) to have reduced expression of CD45RA.

In some embodiments, the immune cells expressing any of the chimeric receptors described herein are administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

A typical amount of cells, i.e., immune cells or hematopoietic cells, administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In one embodiment, the chimeric receptor (e.g., a nucleic acid encoding the chimeric receptor) is introduced into an immune cell, and the subject (e.g., human patient) receives an initial administration or dose of the immune cells expressing the chimeric receptor. One or more subsequent administrations of the agent (e.g., immune cells expressing the chimeric receptor) may be provided to the patient at intervals of 15 days, 14, 13, 12, 11, 10, 9, 8, 7, 6, 5, 4, 3, or 2 days after the previous administration. More than one dose of the agent can be administered to the subject per week, e.g., 2, 3, 4, or more administrations of the agent. The subject may receive more than one doses of the agent (e.g., an immune cell expressing a chimeric receptor) per week, followed by a week of no administration of the agent, and finally followed by one or more additional doses of the agent (e.g., more than one administration of immune cells expressing a chimeric receptor per week). The immune cells expressing a chimeric receptor may be administered every other day for 3 administrations per week for two, three, four, five, six, seven, eight or more weeks.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

In some embodiments, an agent comprising an antigen-binding fragment that binds a cell-surface lineage-specific antigen and a population of hematopoietic cells deficient in the cell-surface lineage-specific antigen. Accordingly, in such therapeutic methods, the agent recognizes (binds) a target cell expressing the cell-surface lineage-specific antigen for targeting killing. The hematopoietic cells that are deficient in the antigen allow for repopulation of a cell type that is targeted by the agent. In some embodiments, the treatment of the patient can involve the following steps: (1) administering a therapeutically effective amount of an agent targeting a cell-surface lineage-specific antigen to the patient and (2) infusing or reinfusing the patient with hematopoietic stem cells, either autologous or allogenic, where the hematopoietic cells have reduced expression of a lineage specific disease-associated antigen. In some embodiments, the treatment of the patient can involve the following steps: (1) administering a therapeutically effective amount of an immune cell expressing a chimeric receptor to the patient, wherein the immune cell comprises a nucleic acid sequence encoding a chimeric receptor that binds a cell-surface lineage-specific, disease-associated antigen; and (2) infusing or reinfusing the patient with hematopoietic cells (e.g., hematopoietic stem cells), either autologous or allogenic, where the hematopoietic cells have reduced expression of a lineage specific disease-associated antigen.

The efficacy of the therapeutic methods using a an agent comprising an antigen-binding fragment that binds a cell-surface lineage-specific antigen and a population of hematopoietic cells deficient in the cell-surface lineage-specific antigen may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen.

In some embodiments, the agent comprising an antigen-binding fragment that binds to the cell-surface lineage-specific antigen and the population of hematopoietic cells IS administered concomitantly.

In some embodiments, the agent comprising an antigen-binding fragment that binds a cell-surface lineage-specific antigen (e.g., immune cells expressing a chimeric receptor as described herein) is administered prior to administration of the hematopoietic cells. In some embodiments, the agent comprising an antigen-binding fragment that binds a cell-surface lineage-specific antigen (e.g., immune cells expressing a chimeric receptor as described herein) is administered at least about 1 day, 2 days, 3 days, 4 days, 5 days, 6 days, 7 days, 8 days, 9 days, 10 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells. In some embodiments, the agent comprising an antigen-binding fragment that binds a cell-surface lineage-specific antigen (e.g., immune cells expressing a chimeric receptor as described herein) is administered multiple times to the subject prior to administration of the hematopoietic cells.

In some embodiments, the hematopoietic cells are administered prior to the agent comprising an antigen-binding fragment that binds a cell-surface lineage-specific antigen (e.g., immune cells expressing a chimeric receptor as described herein). In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the agent comprising an antigen-binding fragment that binds to the cell-surface lineage-specific antigen.

In some embodiments, the agent targeting the cell-surface lineage-specific antigen and the population of hematopoietic cells are administered at substantially the same time. In some embodiments, agent targeting the cell-surface lineage-specific antigen is administered and the patient is assessed for a period of time, after which the population of hematopoietic cells is administered. In some embodiments, the population of hematopoietic cells is administered and the patient is assessed for a period of time, after which agent targeting the cell-surface lineage-specific antigen is administered.

Also within the scope of the present disclosure are multiple administrations (e.g., doses) of the agents and/or populations of hematopoietic cells. In some embodiments, the agents and/or populations of hematopoietic cells are administered to the subject once. In some embodiments, agents and/or populations of hematopoietic cells are administered to the subject more than once (e.g., at least 2, 3, 4, 5, or more times). In some embodiments, the agents and/or populations of hematopoietic cells are administered to the subject at a regular interval, e.g., every six months.

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukaemia, acute lymphoid leukemia, chronic myelogenous leukaemia, acute lymphoblastic leukemia or chronic lymphoblastic leukemia, and chronic lymphoid leukemia.

In some embodiments, the leukemia is acute myeloid leukaemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). CD33 glycoprotein is expressed on the majority of myeloid leukemia cells as well as on normal myeloid and monocytic precursors and has been considered to be an attractive target for AML therapy (Laszlo et al., Blood Rev. (2014) 28(4):143-53). While clinical trials using anti CD33 monoclonal antibody based therapy have shown improved survival in a subset of AML patients when combined with standard chemotherapy, these effects were also accompanied by safety and efficacy concerns.

Other efforts aimed at targeting AML cells have involved the generation of T cells expressing chimeric antigen receptors (CARs) that selectively target CD33 in AML. Buckley et al., Curr. Hematol. Malig. Rep. (2):65 (2015). However, the data is limited and there are uncertainties about how effective (whether all targeted cells are eliminated) this approach may be in treating the patient. Additionally, since myeloid lineage cells are indispensable for life, depleting a subject of myeloid lineage cells could have detrimental effects on survival of the patient. The present disclosure aims at, at least in part, solving such problems associated with AML treatment.

Alternatively or in addition, the methods described herein may be used to treat non-hematopoietic cancers, including without limitation, lung cancer, ear, nose and throat cancer, colon cancer, melanoma, pancreatic cancer, mammary cancer, prostate cancer, breast cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods of the present disclosure include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the present disclosure are used to treat subjects having cancer of the breast, cervix, ovary, prostate, lung, colon and rectum, pancreas, stomach or kidney.

Sarcomas are mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some embodiments, the cancer to be treated can be a refractory cancers. A “refractory cancer,” as used herein, is a cancer that is resistant to the standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, or surgery, or radiation, or a combination thereof. Those of ordinary skill in the art are aware of such standards of care. Subjects being treated according to the present disclosure for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include, but are not limited to, leukemia, melanomas, renal cell carcinomas, colon cancer, liver (hepatic) cancers, pancreatic cancer, Non-Hodgkin's lymphoma and lung cancer.

Any of the immune cells expressing chimeric receptors described herein may be administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### Kits for Therapeutic Uses

Also within the scope of the present disclosure are kits for use of the agents targeting cell-surface lineage-specific antigens in combination with populations of hematopoietic cells that are deficient in the cell-surface lineage-specific antigen. Such kits may include one or more containers comprising a first pharmaceutical composition that comprises any agent comprising an antigen-binding fragment that binds a cell-surface lineage-specific antigen (e.g., immune cells expressing chimeric receptors described herein), and a pharmaceutically acceptable carrier, and a second pharmaceutical composition that comprises a population of hematopoietic cells that are deficient in the cell-surface lineage-specific antigen (e.g., a hematopoietic stem cell) and a pharmaceutically acceptable carrier.

In some embodiments, a kit described herein comprises a gRNA that binds a site having a sequence of ATCCCTGGCACTCTAGAACC (SEQ ID NO: 50) or CCTCACTAGACTTGACCCAC (SEQ ID NO: 58). In some embodiments, the gRNA comprises the nucleotide sequence of CCUCACUAGACUUGACCCAC (SEQ ID NO: 70) or AUCCCUGGCACUCUAGAACC (SEQ ID NO: 67).

In some embodiments, the kit can comprise instructions for use in any of the methods described herein. The included instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the agents targeting cell-surface lineage-specific antigens and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variants as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1: In vitro Deletion of CD33 in a Human Leukemic Cell Line

In order to test the ability of CRSPR-Cas9 system to target CD33 in vitro, human leukemic cells K-562 were co-transfected using Neon™ (Thermo Fisher Scientific) with Cas9-GFP (PX458, S. pyogenes) and a guide RNA containing NGG PAM sequence (FIG. 4) where guide RNA was designed to target hCD33 genomic sequence. 48 hours post-transfection, cells expressing Cas9 were identified and isolated using FACS sorting for GFP. Cells were then incubated for 96 hours and tested for CD33 expression by flow cytometry (FIG. 5). Flow cytometry plots using an anti-CD33 antibody show CD33 expression by the K-562 cells before (top plot) and after (bottom plot) delivery of Cas9 vector and guide RNA. As shown in FIG. 5, 98% of the cells lacked the CD33 expression following transfection.

This example demonstrates the efficient deletion of CD33 using CRISPR-Cas9 system in human leukemic cells.

### EXAMPLE 2: In vitro Deletion of CD45 in Human Leukemic Cell Lines

The CRISPR-Cas9 system was used to target CD45RA in vitro. Briefly, TIB-67 reticulum cell sarcoma mouse macrophage-like cells were co-transfected using Neon™ reagent (Thermo Fisher Scientific) with Cas9-GFP (PX458, S. pyogenes) and CRISPRs gRNAs (containing the “NGG” PAM sequence) targeting hCD45RA genomic sequence. 48 hours post-transfection, cells expressing CRISPR-Cas9 system were identified and isolated using FACS sorting for GFP. Cells were then incubated for 96 hours and tested for CD45RA expression (FIG. 6). Flow cytometry plots using CD45RA antibody show CD45RA expression before (top plot) and after (bottom plot) delivery of Cas9 vector and guide RNA.

Similar to Example 1, where CD33 expression was successfully reduced in leukemic cells, findings in this Example indicate efficient targeting of CD45RA using the CRISPR-Cas9 system.

### EXAMPLE 3: Targeting Cell-surface Lineage-specific CD33 in Acute Myeloid Leukemia (AML)

The present example encompasses targeting of the CD33 antigen in AML. The specific steps of the example are outlined in Table 6.

TABLE 6
Outline of the Experimental Design

| | |
|---|---|
| I. Autologous CD33 | 1. Generation of anti-CD33 CAR constructs |
| targeted (CAR) T-cell | 2. Isolation of CD8⁺T Cells from a Patient |
| therapy | 3. Preparation of anti CD33 CAR T Cells |
| | 4. Reinfusion of CD33 CAR T cells into a |
| | Patient |
| II. Autologous | 1. Isolation of Hematopoietic Stem Cells |
| Hematopoietic Stem | 2. CRISPR-Cas9 Plasmid Targeting CD33 |
| Cell Transplant Using | 3. Generation of CD34⁺CD33⁻ cells via |
| CD34⁺CD33⁻ Cells | CRISPR-CAS System |
| | 4. Reinfusion of CD34⁺CD33⁻ cells into a |
| | Patient |
| III. Continued treatment | |
| of a patient with a CD33 | |
| antibody attached to a | |
| toxin (immunotoxin) | |

### I. CD33-Targeted Chimeric Antigen Receptor (CAR) T-Cell Therapy

A. Generation of Anti-CD33 CAR Constructs

The chimeric antigen receptors targeting CD33 described herein may consist of the following components in order from 5′ to 3′: pHIV-Zsgreen lentiviral backbone (www.addgene.org/18121/), peptide signal, the CD33 scFv, the hinge, transmembrane regions of the CD28 molecule, the intracellular domain of CD28, and the signaling domain of TCR-ζ molecule.

Initially, peptide signal, anti-CD33 light chain (SEQ ID NO: 1), the flexible linker and the anti-CD33 heavy chain (SEQ ID. NO. 2) are cloned into the EcoRI site of pHIV-Zsgreen, with an optimal Kozak sequence.

The nucleic acid sequences of an exemplary chimeric receptors that binds CD33 with the basic structure of Light chain-linker-Heavy chain-Hinge-CD28/ICOS -CD3 ζ is provided below.
Part 1: Light chain-linker—Heavy chain (SEQ ID NO: 16): The Kozak start site is shown in boldface. The peptide signal L1 is shown in italic. The anti-CD33 light chain and heavy chain are shown in bold and italics, separated by a linker.

ggtgtcgtgagcggccgctgaactgGCCACCATGGACATGAGGGTCCCTGCTCAGCTCCTGGGGC
TCCTGCTGCTCTGGCTCTCAGGTGCCAGATGT

[symbol]

[symbol]

[symbol]

[symbol]

[symbol]

[symbol]

[symbol]

[symbol]

[symbol]

[symbol]

[symbol]

[symbol]

[symbol]

GGCAGCACCAGC
GGCAGCGGCAAGCCCGGCAGCGGCGAGGGCAGCACCAAGGGC

[symbol]

[symbol]

[symbol]

[symbol]

[symbol]

[symbol]

[symbol]

[symbol]

[symbol]

[symbol]

[symbol]

[symbol]

[symbol]

[symbol]

Part 2: Hinge-CD28/ICOS—CD3ζ NotI restriction enzyme recognition sites are shown in capitalization. The translational stop site is in boldface. The BamHI restriction cleavage site is shown in underline.

CD28 costimulatory domain
(SEQ ID NO: 17)
GCGGCCGCAattgaagttatgtatcctcctccttacctagacaatgaga
agagcaatggaaccattatccatgtgaaagggaaacacctttgtccaag
tcccctatttcccggaccttctaagcccttttgggtgctggtggtggtt
ggtggagtcctggcttgctatagcttgctagtaacagtggcctttatta
ttttctgggtgaggagtaagaggagcaggctcctgcacagtgactacat
gaacatgactccccgccgccccgggcccAcccgcaagcattaccagccc
tatgccccaccacgcgacttcgcagcctatcgctccagagtgaagttca
gcaggagcgcagacgcccccgcgtaccagcagggccagaaccagctcta
taacgagctcaatctaggacgaagagaggagtacgatgttttggacaag
agacgtggccgggaccctgagatggggggaaagccgagaaggaagaacc
ctcaggaaggcctgtacaatgaactgcagaaagataagatggcggaggc
ctacagtgagattgggatgaaaggcgagcgccggaggggcaaggggcac
gatggcctttaccagggtctcagtacagccaccaaggacacctacgacg
cccttcacatgcaggccctgccccctcgcTAAcgcccctctccctcccc
cccccctaa
ICOS costimulatory domain
(SEQ ID NO: 18)
GCGGCCGCActatcaatttttgatcctcctccttttaaagtaactctta
caggaggatatttgcatatttatgaatcacaactttgttgccagctgaa
gttctggttacccataggatgtgcagcctttgttgtagtctgcattttg
ggatgcatacttatttgttggcttacaaaaaagaagtattcatccagtg
tgcacgaccctaacggtgaatacatgttcatgagagcagtgaacacagc
caaaaaatctagactcacagatgtgaccctaagagtgaagttcagcagg
agcgcagacgcccccgcgtaccagcagggccagaaccagctctataacg
agctcaatctaggacgaagagaggagtacgatgttttggacaagagacg
tggccgggaccctgagatggggggaaagccgagaaggaagaaccctcag
gaaggcctgtacaatgaactgcagaaagataagatggcggaggcctaca
gtgagattgggatgaaaggcgagcgccggaggggcaaggggcacgatgg
cctttaccagggtctcagtacagccaccaaggacacctacgacgccctt
cacatgcaggccctgccccctcgcTAAcgcccctctccctccccccccc
ctaa
Fusion (hybrid) CD28 and ICOS costimulatory
domain
(SEQ ID NO: 19)
GCGGCCGCAattgaagttatgtatcctcctccttacctagacaatgaga
agagcaatggaaccattatccatgtgaaagggaaacacctttgtccaag
tcccctatttcccggaccttctaagcccttttgggtgctggtggtggtt
ggtggagtcctggcttgctatagcttgctagtaacagtggcctttatta
ttttctgggtgaggagtaagaggagcaggctcctgcacagtgactacat
gttcatgagagcagtgaacacagccaaaaaatctagactcacagatgtg
accctaagagtgaagttcagcaggagcgcagacgcccccgcgtaccagc
agggccagaaccagctctataacgagctcaatctaggacgaagagagga
gtacgatgttttggacaagagacgtggccgggaccctgagatgggggga
aagccgagaaggaagaaccctcaggaaggcctgtacaatgaactgcaga
aagataagatggcggaggcctacagtgagattgggatgaaaggcgagcg
ccggaggggcaaggggcacgatggcctttaccagggtctcagtacagcc
accaaggacacctacgacgcccttcacatgcaggccctgccccctcgcT
AAcgcccctctccctcccccccccctaa

In the next step, the hinge region, CD28 domain (SEQ ID NO: 3) and a cytoplasmic component of TCR-ζ are cloned into the NotI and BamHI sites of pHIV-Zsgreen (already containing the peptide signal and the CD33 scFv. Alternatively, CD28 domain can be substituted by ICOS domain (SEQ ID NO: 4).

In addition to CD28 and ICOS domains, a fusion domain comprising fragments of CD28 and ICOS intracellular signaling domains will be engineered (SEQ ID NO: 5) and used to generate additional chimeric receptors. Such configuration, where the chimeric receptor comprises an antigen-binding fragment, an anti-CD33 light chain variable region, a linker, an anti-CD33 heavy chain variable region, CD28/ICOS hybrid region (including a TM region of CD28), and signaling domain of TCR-ζ molecule.

Example amino acid sequences of components that may be used to generate the chimeric receptors are provided herein, such as CD28 domain (SEQ ID NO: 6), ICOS domain (SEQ ID NO: 7), CD28/ICOS hybrid domain (SEQ ID NO: 8), and TCR-t are provided herein. Alternatively, the chimeric receptor may be generated as well (Section B.)

### B. Alternative Method for Generation of Anti-CD33 CAR Constructs

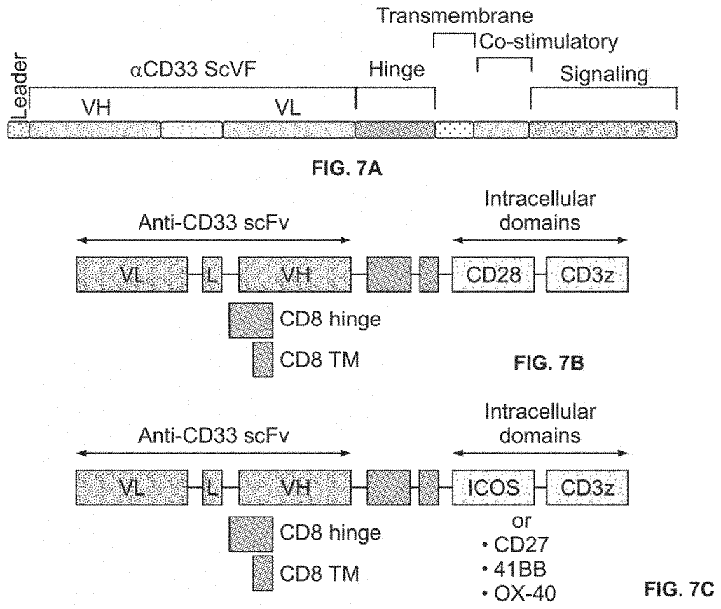

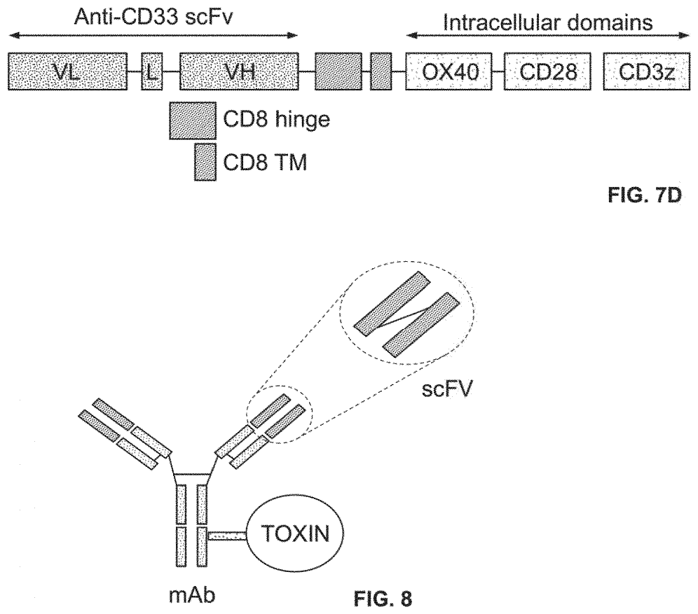

Schematics of example chimeric receptors are presented in FIG. 7, panels A-D. The chimeric receptor will be generated using an extracellular humanized scFv recognizing the CD33 antigen, linked to an extracellular CD8 hinge region, a transmembrane and cytoplasmic signaling domain, and a CD3 ζ-signaling chain (FIG. 7, panel B). DNA encoding the anti-CD33 chimeric receptor will be generated by using a humanized scFv (Es sand et al., J Intern Med. (2013) 273(2):166). Alternatives include a CART cell that contains OX-1 or 41-BB in place of CD28 or CD28/OX1 or CD28/4-1-B-B hybrids (FIG. 7, panels C and D).

In order to generate the anti-CD33 scFV sequence, the coding regions of the heavy and light chains of the variable regions of the anti-CD33 antibody described above (SEQ ID NOs: 1 and 2) will be amplified with specific primers and cloned into a pHIV-Zsgreen vector for expression in cells. To evaluate the binding strength of the scFv (single chain variable fragments) to the target antigen, the scFv will be expressed in Hek293T cells. For this purpose, the vector (pHIV-Zsgreen containing the coding areas) will be transformed into E. coli Top1OF bacteria and the plasmids prepared. The obtained expression vectors that code for the scFv antibodies will be introduced by transfection into Hek293T cells. After culturing the transfected cells for five days, the supernatant will be removed and the antibodies purified.

The resulting antibodies can be humanized using framework substitutions by protocols known in the art. See, for example, one such protocol is provided by BioAtla (San Diego), where synthetic CDR encoding fragment libraries derived from a template antibody are ligated to human framework region encoding fragments from a human framework pool limited to germline sequences from a functionally expressed antibodies (bioatla.com/applications/express-humanization/).

Affinity maturation may be performed in order to improve antigen binding affinity. This can be accomplished using general techniques known in the art, such as phage display (Schier R., J. Mol. Biol (1996), 263:551). The variants can be screened-for their biological activity (e.g., binding affinity) using for example Biacore analysis. In order to identify hypervariable region residues which would be good candidates for modification, alanine scanning mutagenesis can be performed to identify hypervariable region residues contributing significantly to antigen binding. Additionally, combinatorial libraries described by can also be used for improving the affinity of the antibodies (Rajpal et al., PNAS (2005) 102(24): 8466). Alternatively, BioAtla has developed a platform for the rapid and efficient affinity maturation of antibodies, which can also be utilized for the purposes of antibody optimization (bioatla.com/applications/functional-maturation/).

### (C) Assembly of CAR Construct

Next, the anti-CD33 scFv will be linked to an extracellular CD8 hinge region, a transmembrane and cytoplasmic CD28 signaling domain, and a CD3 ζ-signaling chain. Briefly, primers specific for anti-CD33 scFv sequence will be used to amplify the scFv as described above. Plasmid (pUN1-CD8). (www.invivogen.com/puno-cd8a) carrying the complete human CD8 coding sequence will be used to amplify CD8 hinge and transmembrane domains (amino acids 135-205). CD3 ζ fragment will be amplified from the Invivogen plasmid pORF9-hCD247a (http://www.invivogen.com/PDF/pORF9-hCD247a_10E26v06.pdf) carrying the complete human CD3ζ coding sequence. Finally, the CD28 (amino acids 153-220, corresponding to TM and signaling domains of CD28) will be amplified from cDNA generated using RNA collected from activated T cells by Trizol method. Fragments containing anti-CD33-scFv-CD8-hinge+TM-CD28-CD3ζ will be assembled using splice overlap extension (SOE) PCR. The resulting PCR fragment will then be cloned into pELPS lentiviral vector. pELPS is a derivative of the third-generation lentiviral vector pRRL-SIN-CMV-eGFP-WPRE in which the CMV promoter was replaced with the EF-1α promoter and the central polypurine tract of HIV was inserted 5′ of the promoter (Milone et al., Mol Ther. (2009) (8): 1453, Porter et al., NEJM (2011) (8):725). All constructs will be verified by sequencing.

Alternatively, CARs containing ICOS, CD27, 41BB, or OX-40 signaling domain instead of CD28 domain will be generated, introduced into T-cells and tested for the ability to eradicate CD33 positive cells (FIG. 7, panel C). The generation of “third-generation” chimeric receptors are also contemplated (FIG. 7, panel D), which combine multiple signaling domains, such as CD3z-CD28-41BB or CD3z-CD28-0×40, to further augment potency (Sadelain et al., Cancer Discov. (2013) 4:388).

### (D) Anti-CD33 CAR T Cell Preparation

Primary human CD8+T cells will be isolated from patients' peripheral blood by immunomagnetic separation (Miltenyi Biotec). T cells will be cultured in complete media (RPMI 1640 supplemented with 10% heat-inactivated FBS, 100 U/mL penicillin, 100 μg/mL streptomycin sulfate, 10 mM HEPES) and stimulated with anti-CD3 and anti-CD28 mAbs—coated beads (Invitrogen) as previously described (Levine et al., J. Immunol. (1997) 159(12):5921).

A packaging cell line will be used to generate the viral vector, that is able to transduce target cells and contains the anti-CD33 chimeric receptors. To generate lentiviral particles, CARs generated in section (1) of this Example will be transfected into immortalized normal fetal renal 293T packaging cells together with Cells will be cultured with high glucose DMEM, including 10% FBS, 100 U/ml penicillin and 100 μg/ml streptomycin. 48-72 hours post-transfection the supernatant will be collected, and the recombinant lentivirus concentrated in DMEM without FBS. Primary CD8+ T cells will next be transduced at multiplicity of infection (MOI) of ˜5-10 in the presence of polybrene. Human recombinant IL-2 (R&D Systems) will be added every other day (50 IU/mL). T cells will be cultured for ˜14 days after stimulation. Transduction efficiency of human primary T cells will be assessed by expression of a ZsGreen reporter gene (Clontech, Mountain View, Calif.).

E. Infusion of CAR T Cells into a Patient

Prior to the i.v. infusion of anti-CD33 CAR T cells into the patient, cells will be washed with phosphate buffered saline and concentrated. A cell processor such as a Haemonetics CellSaver (Haemonetics Corporation, Braintree, Mass.), which provides a closed and sterile system, will be used for the washing and concentration steps before formulation. The final T cells expressing the anti-CD33 chimeric receptors will be formulated into 100 ml of sterile normal saline supplemented with human serum albumin. Finally, patients will be infused with 1-10×10⁷ T cells/kg over a period of 1-3 days (Maude et al., NEJM (2014) 371(16):1507). The number of T cells expressing anti-CD33 chimeric receptors infused will depend on numerous factors such as the state of the cancer patient, patient's age, prior treatment, etc.

Furthermore, also contemplated herein are immune cells expressing chimeric receptors that target CD45RA in addition to chimeric receptors that target CD33 in AML patients. This can be accomplished by two different approaches: 1) generating immune cells expressing anti-CD33 chimeric receptors and immune cells expressing anti-CD47RA chimeric receptors separately and infusing the patient with both types of immune cells separately, or 2) generating immune cells that target both CD33 and CD45RA simultaneously (Kakarla et al., Cancer (2):151 (2014)).

II. Autologous Hematopoietic Stem Cell Transplant (HSCT) Using CD34⁺CD33⁻ Cells

It is understood that the protocols regarding stem cell isolation from patients, conditioning regimens, as well as infusion of patients with stem cells vary greatly depending on the patient's age, condition, treatment history, and institution where the treatment is conducted. Thus, the protocol described below is merely an example and is subject to routine optimization by a person having ordinary skill in the art.

A. Isolation of Hematopoietic Stem Cells Using Peripheral Blood Stem Cell (PBSC) Mobilization Following Adoptive Transfer of anti-CD33 CAR T Cells

AML patient will be stimulated by i.v. administration of granulocyte colony-stimulating factor (G-CSF) 10 mg/kg per day. CD34⁺ cell positive selection will be performed using immunomagnetic beads and an immunomagnetic enrichment device. A minimum of 2×10⁶ CD34⁺ cells/kg body weight are expected to be collected using a Fenwall CS 3000+ cell separator (Park et al., Bone Marrow Transplantation (2003) 32:889).

B. Conditioning Regimen of a Patient

The conditioning regimen for autologous peripheral blood stem cell transplant (PBSCT) will be carried out using etoposide (VP-16)+cyclophosphamide (CY)+total body irradiation (TBI). Briefly, the regimen will consists of etoposide (VP-16) at 1.8 g/m² i.v. constant infusion (c.i.v.) over 26 h as a single dose followed by cyclophosphamide (CY) at 60 mg/kg per day i.v. over 2 h for 3 days, followed by total body irradiation (TBI) at 300 cGy per day for the next 3 days.

To calculate the dose, ideal body weight or actual body weight, whichever is less, will be used. As previously mentioned, factors such as the state of the cancer patient, patient's age, prior treatment, as well as the type of institution where the procedure is conducted will all be taken into consideration when determining the precise conditioning regimen.

C. Plasmid Construction of CRISPR-Cas9 System targeting CD33

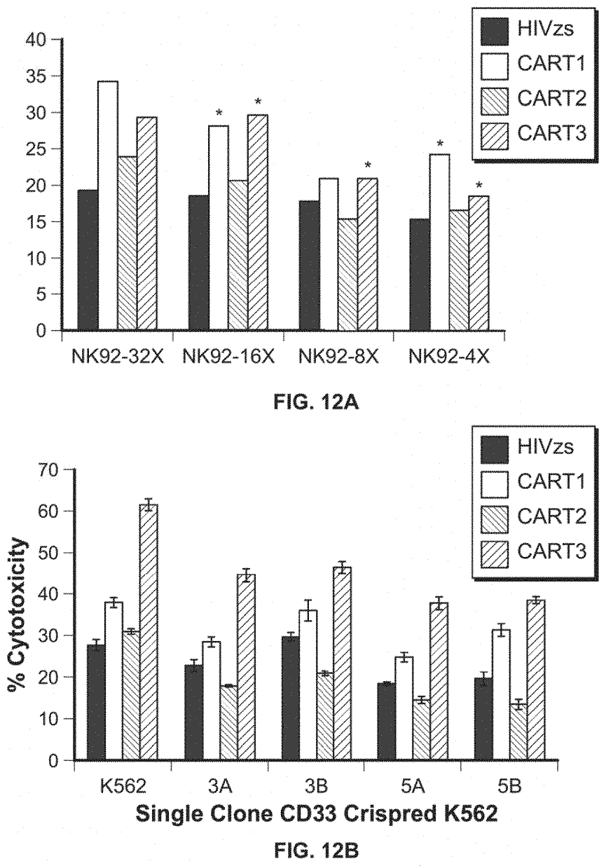

The lentiCRISPR v2 containing inserts Cas9 and Puromycin resistance will be obtained from Addgene (Plasmid #52961) (Sanjana et al., Nat Methods (2014) (8):783). To clone the single guide RNA (sgRNA) CD33 guide sequence, the lentiCRISPR v2 will be cut and dephosporylated with FastDigest BsmBI and FastAP (Fermentas) at 37° C. for 2 hours. gRNA targeting CD33 will be designed using the online optimized design tool at crispr.mit.edu. Alternatively, gRNA will have a sequence depicted in FIG. 12 (SEQ ID NO:11). CD33 gRNA oligonucleotides will be obtained from Integrated DNA Technologies (IDT), phosphorylated using polynucleotide kinase (Fermentas) at 37° C. for 30 minutes and annealed by heating to 95° C. for 5 minutes and cooling to 25° C. at 1.5° C./minute. T7 ligase will be used to anneal the oligos, after which the annealed oligos will be ligated into gel purified vector (Qiagen) at 25° C. for 5 minutes. Resulting plasmid can then be amplified using an endotoxin-free midi-prep kit (Qiagen) (Sanjana et al., Nat Methods (2014)(8):783).

Alternatively, a two vector system may be used (where gRNA and Cas are expressed from separate vectors) protocol described previously (Mandal et al., Cell Stem Cell (2014) 15(5):643). Here, Mandal et al. achieved efficient ablation of genes in human hematopoietic stem cells using CRISPR-Cas system expressed from non-viral vectors.

Briefly, human-codon-optimized Cas9 gene containing a C-terminal SV40 nuclear localization signal will be cloned into a CAG expression plasmid with 2A-GFP. To direct Cas9 to cleave CD33 sequences of interest, the guide RNA (gRNA (SEQ ID. NO. 11)) will be separately expressed from a plasmid containing the human U6 polymerase III promoter. gRNA sequence oligonucleotides will be obtained from Integrated DNA Technologies (IDT), annealed, and introduced into the plasmid using BbsI restriction sites. Due to the transcription initiation requirement of a ‘G’ base for human U6 promoter, as well as the requirement for the PAM (protospacer-adjacent motif) sequence, genome target will comprise GN₂₀GG nucleotide sequence.

In addition to infusing patients with CD33 depleted hematopoietic stem cells HSCs, a protocol will be developed in which the patients are subsequently infused with CD45RA depleted HSCs. Alternatively, the inventors will generate CD34⁺CD33⁻CD45RA⁻ cells using CRISPR-Cas9 system to reduce both CD33 and CD45RA genes simultaneously. Example guide RNA sequences for CD45RA and CD33 are shown in Tables 4 and 5).

D. Transfection of CD34⁺ Cells HSCs to Generate CD34⁺CD33⁻ Cells

Freshly isolated peripheral blood-derived CD34⁺ cells (from step 4) will be seeded at 1×10⁶ cells/ml in serum-free CellGro SCGM Medium in the presence of cells culture grade Stem Cell Factor (SCF) 300 ng/ml, FLT3-L 300 ng/ml, Thrombopoietin (TPO) 100 ng/ml and IL-3 60 ng/ml. Following 24 hour of pre-stimulation, CD34⁺ HSCs will be transfected with LentiCRISPR v2 containing Cas9 and CD33 gRNA using Amaxa Human CD34 cell Nucleofector kit (U-008) (#VPA-1003) (Mandal et al., Cell Stem Cell (2014) 15(5):643). 24-48 hours post-transfection, CD34⁺ CD33⁻ cells are selected with 1.2 ᵢig/m1puromycin. Following the puromycin selection, CD34⁺ CD33⁻ cells will be maintained in puromycin-free media for couple of days.

E. Reinfusion of CD34⁺CD33⁻ Cells into the Patient

CD34⁺ cells transfected ex vivo with CRISPR-Cas9-CD33 (CD34⁺CD33⁻ cells) are immediately reinfused through a Hickman catheter using a standard blood administration set without a filter (Hacein-Bey Abina et al. JAMA (2015) 313(15):1550).

Generally, patients who have undergone the above outlined treatment protocol will be monitored for the reappearance of circulating blasts and cytopenias. Additionally, depending on the underlying mechanism of AML in a specific patient, the success of the treatment will be monitored by testing for reappearance of an informative molecular or cytogenetic marker, or an informative flow cytometry pattern. For example, reemergence of a BCR-ABL signal in Philadelphia chromosome-positive AML will be detected using fluorescent in situ hybridization (FISH) with probes for BCR (on chromosome 22) and ABL (on chromosome 9).

To evaluate the success of CD33 deletion via CRISPR-Cas9 system, peripheral blood CD34⁺ cells will be isolated from patients (post-transplant) and assessed for the CD33 expression, for example using flow cytometry, Western blotting, or immunohistochemistry.

As described herein, the HSCT described in this Example can be either autologous or allogeneic, and both approaches are suitable and can be incorporated in the methods described in the present disclosure.

III. Optional Step: Continued Treatment of a Patient with a CD33 Antibody Attached to a Toxin

A. Treatment of Patients with CD33 Immunotoxin Gemtuzumab Ozogamicin (GO)

Patients will be treated with 9 mg/m² of anti-CD33 antibody gemtuzumab ozogamicin (GO) as a 2-hour intravenous infusion in 2 doses separated by 2 weeks (Larson et al., Cancer (2005), 104(7):1442-52). GO is comprised of a humanized monoclonal antibody against CD33 which is conjugated with a cytostatic agent, calicheamicin (FIG. 8).

Alternatively, the anti-CD33 antibodies may be conjugated to different toxins, such as diphtheria toxin, Pseudomonas exotoxin A (PE), or ricin toxin A chain (RTA) can be generated (Wayne et al., Blood (2014) 123(16): 2470). Similarly, anti-CD45RA antibodies may be attached to a toxin and included in the treatment regimen.

### EXAMPLE 4: T cells and NK Cell Lines Expressing an Anti-CD33 Chimeric Receptor Induce Cell Death of Target Cells Expressing CD33

### Binding of Chimeric Receptors to CD33

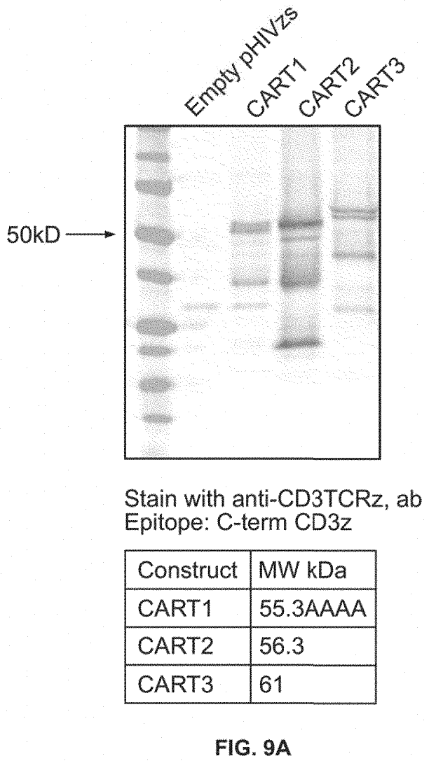

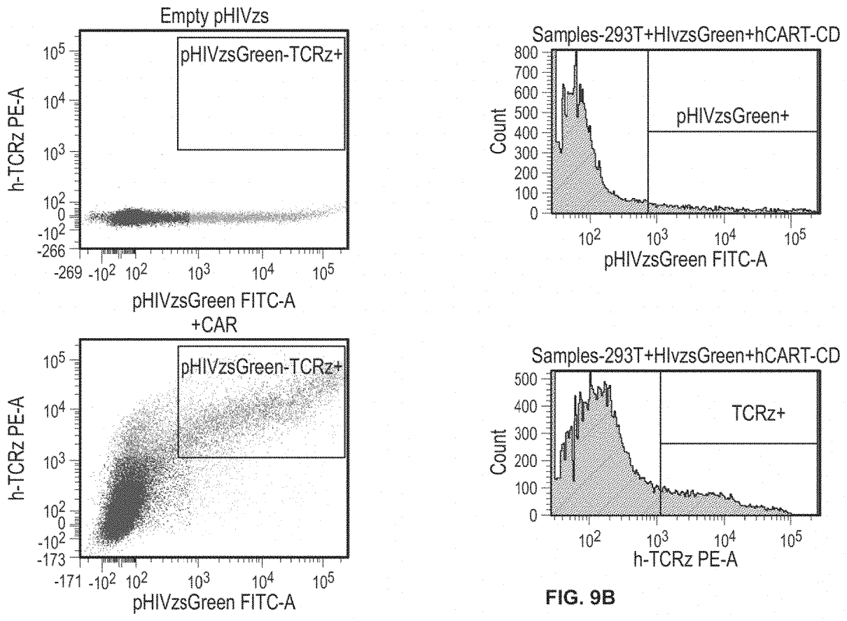

Chimeric receptors that bind CD33 (e.g., CART1, CART2, CART3) were generated using convention recombinant DNA technologies and inserted into a pHIV-Zsgreen vector (Addgene; Cambridge, Mass.). The vectors containing the chimeric receptors were used to generate lentiviral particles, which were used to transduce different cell types, for example T cell lines (e.g., 293 T cells) and NK cell lines (e.g., NK92 cells). Expression of the chimeric receptors was detected by Western blotting (FIG. 9, panel A) and flow cytometry (FIG. 9, panel B).

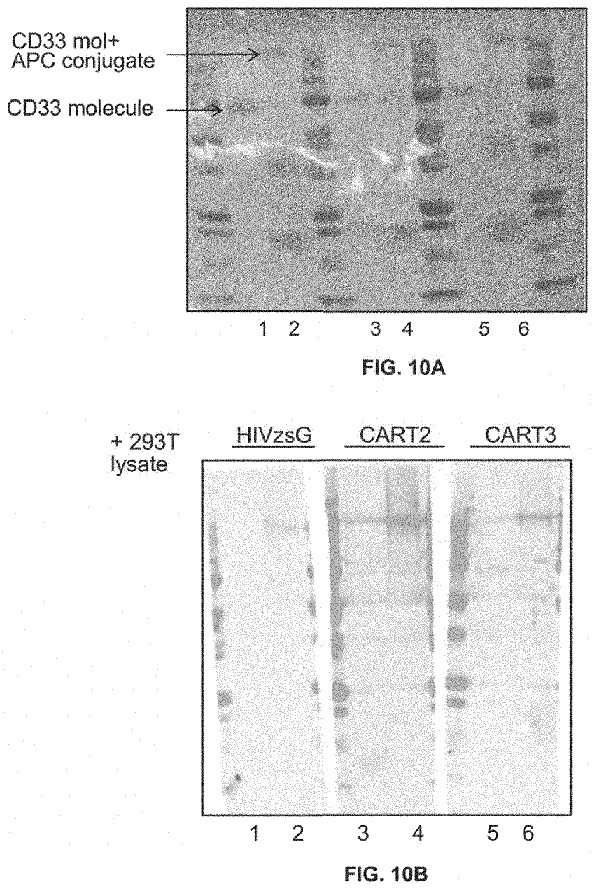

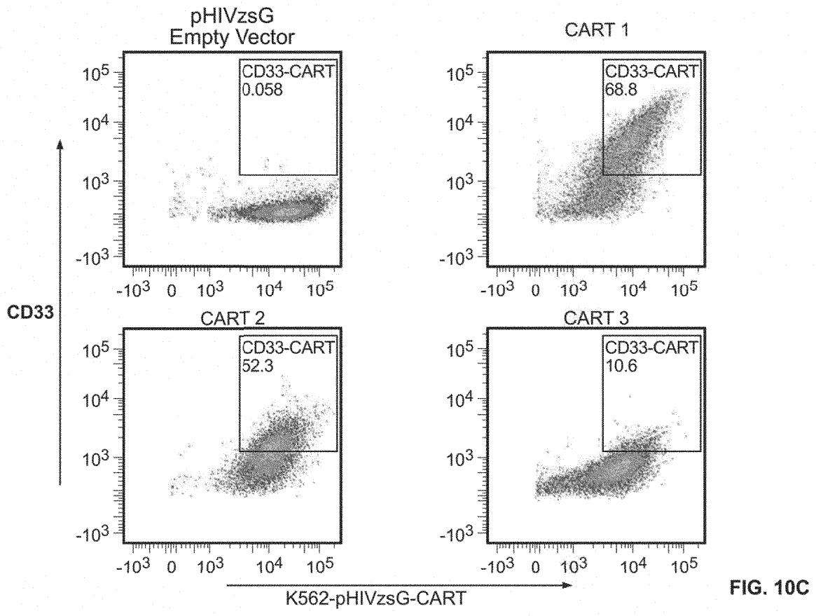

Cells expressing the chimeric receptors were selected by fluorescence-activated cell sorting (FACS) and assessed for their ability to bind CD33. Briefly, lysates of 293T cells expressing the chimeric receptors were coincubated with CD33 or CD33-allophycocyanin (APC) conjugate. The samples were subjected to protein electrophoresis and either stained with Ponceau protein stain (FIG. 10, panel A) or transferred to a membrane and probed with an anti-CD3t primary antibody (FIG. 10, panel B). In both cases, binding between the chimeric receptors and their target, CD33.

K562 cells expressing the chimeric receptors were also assessed for binding to CD33 by flow cytometry using CD33 as a probe (FIG. 10, panel C). There was an increase in the number of cells positive for expression of the chimeric receptor (CART1, CART2, or CART3) and CD33 binding as compared to cells containing an empty vector control, indicating the chimeric receptors bind to CD33.

### Cytotoxicity Induced by Cells Expressing the Chimeric Receptors

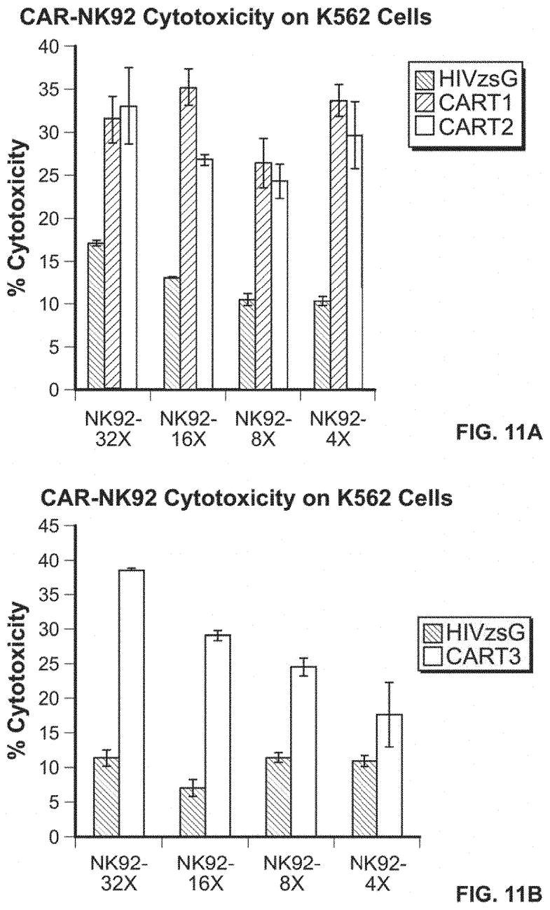

NK-92 cells expressing the chimeric receptors were functionally characterized for the ability to induce cytotoxicity of target cells presenting CD33 on the cell surface (e.g., K562 are a human chronic myelogenous leukemia cell line that are CD33+). To perform the cytotoxicity assays, effector cells (immune cells, such as NK-92 cells) were infected with lentivirus particles encoding the chimeric receptors and expanded. Seven days post infection, cells expressing the chimeric receptors were selected by FACS analysis by selecting for fluorescent markers also encoded by the chimeric receptor encoding vector (e.g., GFP+or Red+). The selected cells that express the chimeric receptors were expanded for one week. Fourteen days post infection, the cytotoxicity assay was performing involving staining the target cells (cells expressing the target cell-surface lineage-specific antigen, CD33) with carboyxfluorescein succinimidyl ester (CFSE) and counting both the target cells and cells expressing the chimeric receptors. Different ratios of target cells and cells expressing the chimeric receptors were coincubated in round bottom 96-well plates for 4.5 hrs, after which 7-aminoactinomycin D (7-AAD) was added to stain non-viable cells. Flow cytometry was performed to enumerate the population of viable and non-viable target cells. As shown in FIG. 11, panels A and B, NK92 cells expressing chimeric receptors CART1, CART2, or CART3 induced a substantial amount of cell death of target K562 cells at each of the cell ratios assessed.

To determine that the cell death of K562 cells was dependent on specific targeting of the chimeric receptor to CD33, K562 were genetically engineered to be deficient in CD33 using a CRISPR/Cas system. Briefly, a human codon-optimized Cas9 endonuclease and a gRNA targeting a portion of the IgC domain of CD33 were expressed in the K562 cells, resulting in populations of CD33-deficient K562 cells. The cells were expanded and co-incubated with NK92 cells expressing the chimeric receptors, and the cytotoxicity assay was performed as described above. As shown in FIG. 12 panel A, the pooled CD33-deficient K562 cells showed a modest reduction in cell death with co-incubated with the NK92 cells expressing the chimeric receptors. However, when single clones of CD33-deficient K562 cells were isolated, expanded, and used to perform the cytotoxicity assays, a more significant reduction in cytotoxicity was observed (FIG. 12, panel B).

### Expression of Chimeric Receptors in Primary T Cells

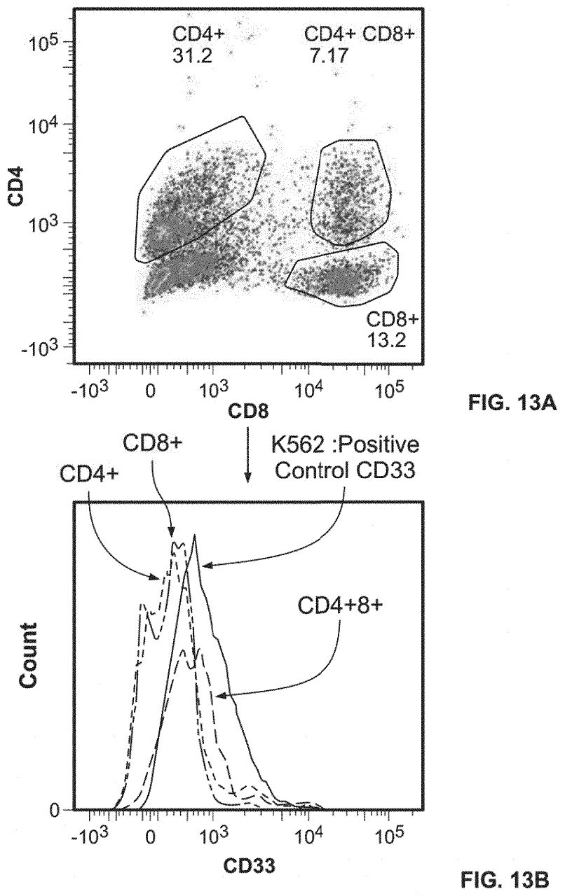

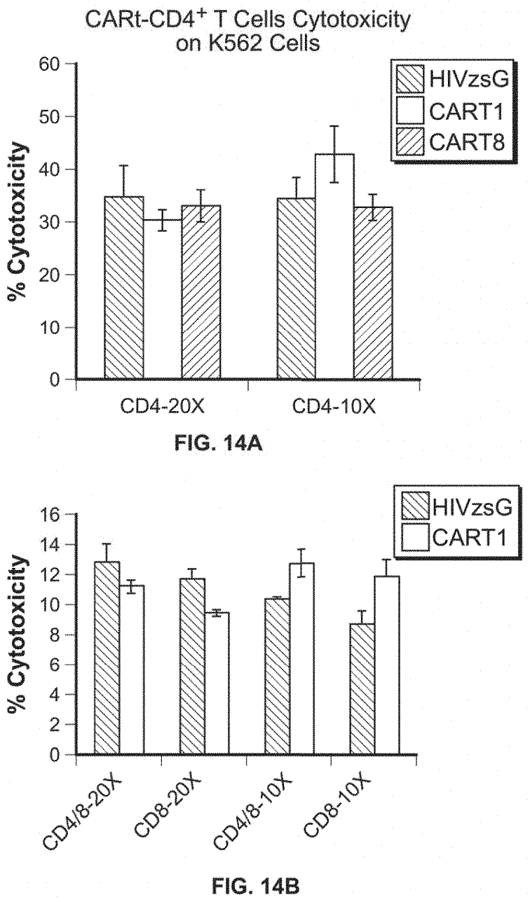

Primary T cell populations were isolated from PMBCs obtained from donors by FACS by positively selecting CD4+, CD8+, or CD4+/CD8+ cells, resulting in highly pure populations (FIG. 13, panels A and B). Each of the populations of primary T cells (CD4+, CD8+, or CD4+/CD8+ cells) were transduced with a lentiviral vector containing the chimeric receptors (e.g., CART1 and CARTE) and the resulting primary T cells expressing chimeric receptors were used to perform cytotoxicity assays, as described above. Co-incubation of the population of CD4+T cells expressing the chimeric receptors with K562 (1000 target K562 cells) did not result in cytotoxicity of the K562 cells (FIG. 14, panel A). In contrast, in a cytotoxicity assay using either CD8+or CD4+/CD8+ cells expressing the chimeric receptors and 1000 target K562 cells, the CD8+or CD4+/CD8+ cells were able to induce cell death of the K562 cells at a low cellular ratio (FIG. 14, panel B).

### Genetic Engineering Human Hematopoietic Stem Cells

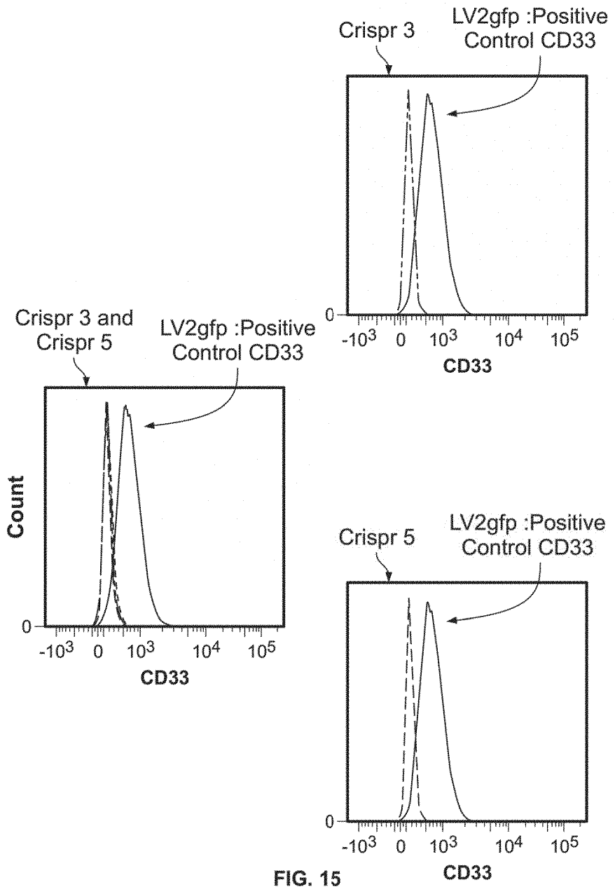

Several gRNAs were designed to hybridize to the IgC domain of CD33 (see, for example, Table 4, SEQ ID NO: 11 or 28-31). Each of the gRNAs were expressed along with a Cas9 endonclease in K562 cells. The expression of CD33 was assessed by flow cytometry (FIG. 15). As shown for Crispr 3 (SEQ ID NO: 28) and Crispr5 (SEQ ID NO: 29), a significant reduction in CD33 was found in cells expressing the CD33-targeting CRISPR/Cas system, as compared to control cells expressing CD33.

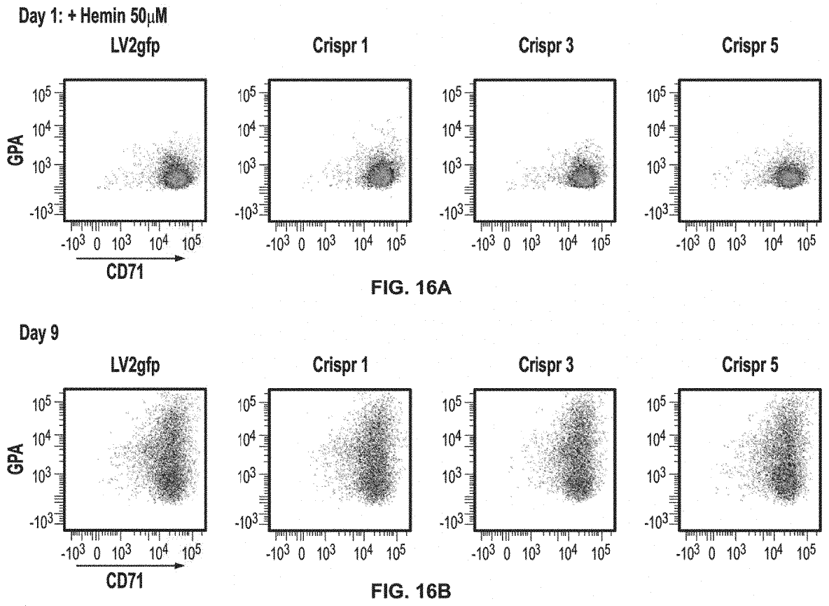

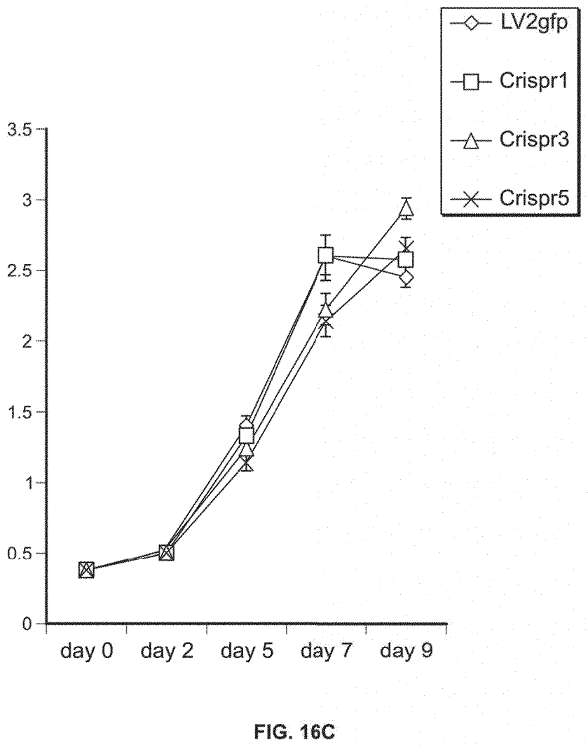

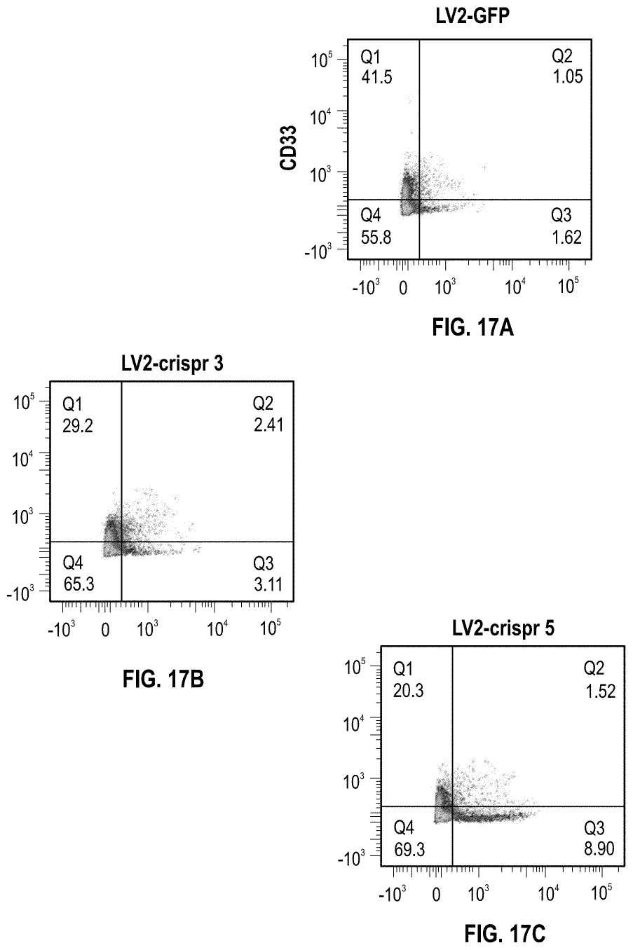

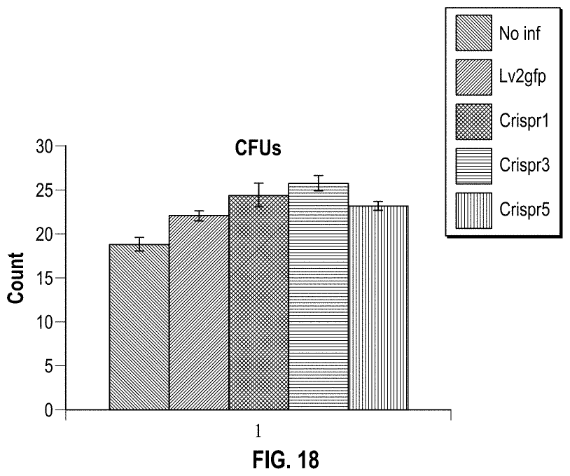

The CD33-deficient hematopoietic stem cells were also assessed for various characteristics, including proliferation, erythropoeitic differentiation, and colony formation. Briefly, CD33-deficient hematopoietic stem cells and control cells were induced to differentiate by exposing the cells to hemin, and CD71, a marker of erythroid precursors, was assessed by flow cytometry at different time points (FIG. 16, panels A and B). CD33-deficient hematopoietic stem cells underwent erythropoeitic differentiation and flow cytometric profiles appeared similar to the control cells (CD33+). The cells were also subjected to MTT assay to measure the metabolic activity of the CD33-deficient hematopoietic stem cells. As shown in FIG. 16, panel C, the CD33-deficient hematopoietic stem cells performed comparably to the control cells. Finally, the ability of the cells to proliferate and form colonies of cells was observed using a microscopic colony formation assay. Again, the CD33-deficient hematopoietic stem cells were able to form colonies to a similar extents as the control cells (FIG. 18). These results indicate the CRISPR/Cas deletion of a portion of CD33 does not significantly impact the ability of the cells to proliferate, differentiate, or form colonies.

### EXAMPLE 5: Gene-Edited Stem Cells Enable CD33-Directed Immune Therapy for Myeloid Malignancies

Antigen-directed immunotherapies for acute myeloid leukemia (AML), such as chimeric antigen receptor T cells (CAR-Ts) or antibody-drug conjugates (ADCs), are associated with severe toxicities due to the lack of unique targetable antigens that can distinguish leukemic cells from normal myeloid cells or myeloid progenitors. Here, a novel approach is presented to treat AML by targeting the lineage specific myeloid antigen CD33. The present approach combines CD33-targeted CAR-T cells and/or the ADC, Gemtuzumab Ozogamicin, with the transplantation of hematopoietic stem cells (HSCs) that have been engineered to ablate CD33 expression using genomic engineering methods. Highly efficient genetic ablation of CD33 antigen using CRISPR/Cas9 technology in human stem/progenitor cells (HSPC) is shown and evidence is provided that the deletion of CD33 in HSPC does not impair their ability to engraft and to repopulate a multilineage hematopoietic system in vivo. Whole genome sequencing and RNA sequencing analysis revealed no detectable off-target mutagenesis and no loss of functional p53 pathways. Using a human AML cell line (HL-60), a post remission marrow with minimal residual disease was modeled and it was shown that the transplantation of CD33-ablated HSPCs with CD33-targeted immunotherapy leads to leukemia clearance, without myelosuppression, as demonstrated by the engraftment and recovery of multilineage descendants of CD33-ablated HSPCs. The present study thus contributes to the advancement of targeted immunotherapy and could be easily replicated in other malignancies.

Acute myeloid leukemia is a disease with unmet need for effective therapies, especially in post-remission patients. Immunotherapy directed against a lineage-specific antigen (LSA) such CD33 show on-target effects but are limited by toxicities because normal myeloid cells and hematopoietic progenitors also express CD33. Here it is shown that genetically ablating CD33 in HSPC, using CRISPR methods, enables immunotherapy against leukemias using anti-CD33 CAR-T or antibody therapy. A post-remission human marrow with minimal leukemic disease in mice is modeled and effective clearance of AML and the reconstitution of the CD33 deleted human graft is shown. This study presents a novel approach to treat myeloid leukemias and could be extended to other cancers and other antigens.

### Material and Methods

### Cell Line and Primary Human Cells

The immortalized human acute myeloid cell line, HL-60 was obtained from ATCC and cultured in IMDMEM with 20% Fetal Bovine Serum and 1% Penicillin Streptomycin. In order to follow leukemia engraftment and progression over time, the HL-60 cells were transduced with lentiviral particles expressing a dTomato fluorescent protein under an EF1α promoter. The lentivirus vector and particles were produced by Vectalys (Toulouse, France). Human Bone Marrow or Cord Blood CD34⁺ stem cells were purchased from StemExpress (Folsom, Calif., USA) and maintained in StemSpan SFEM II (STEMCELL Technologies inc) containing 1% Penicillin Streptomycin, 100 ng/mL TPO, 100 ng/mL SCF, 100 ng/mL IL6 and 100 ng/mL FLT3L and UM171 0.35 nM (Xcessbio, San Diego, Calif., USA). All human cytokines were purchased from Biolegend (San Diego, Calif., USA).

Human T cells were purified from fresh peripheral blood normal donor leukopaks purchased from the New York Blood Center. Briefly, the leukopak was diluted with 2-4 volumes of Phosphate Buffered Saline (1×) supplemented with 2 mM EDTA, store at 4° C. Then 35 mL of diluted leukopak was carefully layered on 15 mL of Ficoll-Paque™ Premium (GE) and centrifuged at 400 g, 30 mins at 25° C., in swinging rotor buckets. The layer of mononuclear cells was then transferred to a new tube, diluted 1:1 with PBS (1×) containing 2 mM EDTA and centrifuged 400 g, 15 mins at 25° C. The red blood cells of the pellet were then removed with 1× ACK lysis buffer (Gibco), incubated 5-8 mins at RT, washed with PBS (1×) containing 2 mM EDTA and centrifuged again 400 g, 10 mins at 25° C. The CD4+ and CD8+ T cells were then positively selected from the mono nuclear cells pellet with Miltenyi Biotec CD4+ and CD8⁺ microbeads, following manufacturer protocols. The CD4+ and CD8+ T cells were then activated the same day using CD3/CD28 dynabeads 1:1 bead to cell ratio (Gibco) and expanded separately in the Gibco OpTmizer™ CTS™ T-Cell Expansion SFM medium containing IL7 10 ng/mL and IL15 5 ng/mL.

### CAR Constructs, Lentiviral Production, and Transduction

The anti-CD33 Chimeric Antigen Receptor was generated by cloning the light and heavy chain of the humanized anti-human CD33 scFv (clone My96) fused in frame, to the CD8 alpha hinge domain, the CD8 transmembrane domain, the 4-1BB signaling domain and the CD3zeta intracellular domain into the lentiviral plasmid pHIV-Zsgreen, a gift from Bryan Welm & Zena Werb (Addgene plasmid # 18121) (Welm et al. Cell Stem Cell. 2008;2(1):90-102). All cDNA fragments were codon optimized and synthesized by GeneArt (Regensburg, Germany). The lentiviral particles were produced by Vectalys (Toulouse, France). 24 hours after activation, CD4+T cells were transduced with lentiviral particles at an MOI=30 and in parallel, CD8+T cells at an MOI=40.

### CRISPR/Cas9 Mediated CD33 Genomic Targeting

Human Bone Marrow or Cord Blood CD34⁺ stem cells were maintained in StemSpan SFEM II (STEMCELL technologies inc) containing 1% Penicillin Streptomycin, and the following human cytokines 100 ng/mL TPO, 100 ng/mL SCF, 100 ng/mL IL6 and 100 ng/mL FLT3L and UM171 0.35 nM (Xcessbio, San Diego, Calif., USA). All human cytokines were purchased from Biolegend (San Diego, Calif., USA).

The TrueCut Cas9 protein V2 was purchased from Invitrogen. The chemically modified sgRNA targeting CD33 were designed using Synthego CRISPR Gene KO design tool and purchased from Synthego. 3 μg TrueCut Cas9 protein and 1.5 μg sgRNA for 200,000 CD34⁺ cells were mixed in P3 buffer (Lonza, Amaxa P3 Primary Cell 4D-Nucleofector Kit) and incubated for 10 mins at 37° C. The cells were then washed with PBS, resuspended in P3 buffer, mixed with the Cas9/sgRNA RNP complex and then electroporated with the 4D-Nucleofector. After electroporation, cells were cultured at 37° C. until analysis.

Deletion efficiency was assessed 7 days after electroporation using the following antibodies from Biolegend: hCD34-PerCp/Cy5.5 and hCD33-FITC.

### Whole Genome and RNA Sequencing

After electroporation with Cas9 only or RNP complex Cas9/sgRNA, CD34⁺ cells were kept in vitro for 10 days and their DNA or RNA isolated as followed. DNA was purified with QIAAmp DNA mini kit, following manufacturer's protocol, then eluted with 30 ul and DNA concentration measured using Nanodrop and Qubit dsDNA BR assay. RNA was purified with a miRNeasy micro kit, following manufacturer's protocol, then eluted with 18 ul. Nanodrop and Bioanalyzer Pico chip assay were performed to measure concentration and quality.

For whole genome sequencing, NEBNext® Ultra™ II DNA Library Prep Kit was used for Illumina, clustering, and sequencing reagents. Briefly, the genomic DNA was fragmented by acoustic shearing, cleaned up and end repaired. Adapters were ligated and DNA libraries were made. The DNA libraries were also quantified by real time PCR (Applied Biosystems, Carlsbad, Calif., USA), clustered on two lanes of a flowcell, and loaded on the Illumina HiSeq instrument according to manufacturer's instructions. The samples were sequenced using a 2× 150 paired-end (PE) configuration. Image analysis and base calling were conducted by the HiSeq Control Software (HCS) on the HiSeq instrument. DNA sequences were processed using Illumina HiSeq Analysis Software v2.1 (HAS 2.1) using default parameters.

For RNA sequencing, cDNA synthesis and amplification were performed using SMART-Seq v4 Ultra Low Input Kit for Sequencing (Clontech, Mountain View, Calif.). The sequencing library was prepared using Nextera XT (Illumina). The samples were sequenced using a 2×150 Paired End (PE) configuration. After investigating the quality of the raw data, the trimmed reads were mapped to the Homo sapiens reference genome available on ENSEMBL using the STAR aligner v.2.5.2b. BAM files were generated as a result of this step. Unique gene hit counts were calculated by using feature Counts from the Subread package v.1.5.2. Only unique reads that fell within exon regions were counted. After extraction of gene hit counts, the gene hit counts table was used for downstream differential expression analysis using the edgeR package within the SARTools package (Varet et al. PLoS One. 2016;11(6):e0157022). Genes were considered significantly differentially expressed if the p-value is >0.05.

### Flow Cytometry and Sorting

### In Vitro

After transduction, CAR-T cells were expanded up to 15 days then sorted for GFP+ using the Biorad S3e sorter (dead cells were excluded using Propidium Iodide) and comixed 1:1 for in vitro and in vivo experiments. CAR expression and their ability to recognize and bind CD33 was assessed by incubating CAR-T cells with biotinylated human CD33 protein (ACRO biosystem) 20 mins at 4° C. and then stained with fluorochrome conjugated streptavidin.

Human CD34⁺stem cells were analyzed 5 to 7 days after electroporation using the following antibodies from Biolegend: hCD34-PerCp/Cy5.5 and hCD33-FITC.

### In Vivo

Engraftment and repopulation of the hematopoietic system over time, was assessed by analysis of peripheral blood, bone marrow aspiration, whole bone marrow (from sacked mice) using the consequent antibodies from Biolegend (San Diego, Calif., USA) or BD Biosciences (San Jose, Calif., USA): Ter119-PeCy5, Ly5-BV711, H2kd-BV711, hCD45-BV510, hCD3-Pacific Blue, hCD123-BV605, hCD33-APC, hCD14-APC/Cy7, hCD10-BUV395, hCD19-BV650, CD34-BV421, CD9O-PeCy7, hCD38-BUV661, and hCD45RA-BUV737. CAR-T cells stably express fluorescent protein zsGreen, leukemic cells stably express dTomato and dead cells were excluded using Propidium Iodide. Leukemia cells were gated on Ter119⁻dtomato⁺. CD34⁺ injected derived human cells were gated on Ter119⁻dtomato⁻ Ly5⁻/H2kd⁻human CD45⁺CART⁻. All data were acquired with the BioRad ZE5 flow cytometry analyzer in high-throughput mode and analysis was performed using FlowJo 10.4.2. Concomitantly, leukemia progression was also assessed by fluorescent imaging using the PerkinElmer IVIS Spectrum Optical Imaging System. Images were acquired and analyzed with Living Image 4.4 Optical Imaging Analysis Software.

### In Vitro Cytotoxicity Assays

Effector sorted CAR-T cells stably expressing zsGreen were mixed at a different ratio with the following target cells HL-60 stably expressing dTomato and or CD34⁺CD33^{WT} cells stained with Celltrace blue and or CD34⁺CD33^{Del} stained with Celltrace Violet (Invitrogen). 16 to 24 hours after incubation, using 7AAD or Sytox Red as a viability dye, data were acquired with the BioRad ZE5 flow cytometry analyzer in high-throughput mode in order to assess cytotoxicity. After subtracting the spontaneous lysis in negative control, CART33 cells specific cytotoxicity (%) was calculated as cells positive for both CFSE and 7-AAD or Sytox Red with the following formula: ((% positive cells with CART33)−(% positive cells with control T cells))/(100−((% positive cells with control T cells))×100.

### In Vivo Experiments

NOD.Cg-Prkdc^{scid} Il2rg^{tm1Wjl} Tg(CMV-IL3,CSF2,KITLG)1Eav/MloySzJ (NSG-SGM3) mice (The Jackson Laboratory, Bar Harbor, Maine, USA) were conditioned with sublethal (1.2 Gy) total-body irradiation (TBI). Human CD34⁺CD33^{Del} Bone Marrow or Cord Blood stem cells (5*10⁵-1*10⁶) along with 5*10⁵dTomato-HL-60 cells were injected intravenously into the mice within 8-24 hours post-TBI. One to two weeks later, mice were treated with 2 to 3*10⁶ anti-CD33 or control CAR-T cells (premixed CD4:CD8=1:1), or 6 μg of GO (Gemtuzumab Ozagamicin) or PBS intravenously injected.

Engraftment and repopulation of the hematopoietic system over time, was assessed by analysis of peripheral blood, bone marrow aspiration, and whole bone marrow (from sacked mice) using the consequent antibodies from Biolegend (San Diego, Calif., USA) or BD Biosciences (San Jose, Calif., USA): Ter119-PeCy5, Ly5-BV711, H2kd-BV711, hCD45-BV510, hCD3-Pacific Blue, hCD123-BV605, hCD33-APC, hCD14-APC/Cy7, hCD10-BUV395, hCD19-BV650, CD34-BV421, CD9O-PeCy7, hCD38-BUV661, and hCD45RA-BUV737. Dead cells were excluded using Propidium Iodide. CD34⁺ injected derived human cells were gated on Ter119⁻, Ly5⁻/H2kd⁻ human CD45⁺.

All experiments were performed under protocols approved by the Institutional Animal Care and Use Committee of Columbia University.

### Statistics

All statistics were performed using Graphpad Prism 7. For continuous variables, an unpaired two tailed t-test was performed. Differences between means were considered significant when the p value is <0.05, else not significant (ns; p>0.05).

### Results

### CRISPR/Cas9 Mediated Genetic Ablation of CD33 Antigen

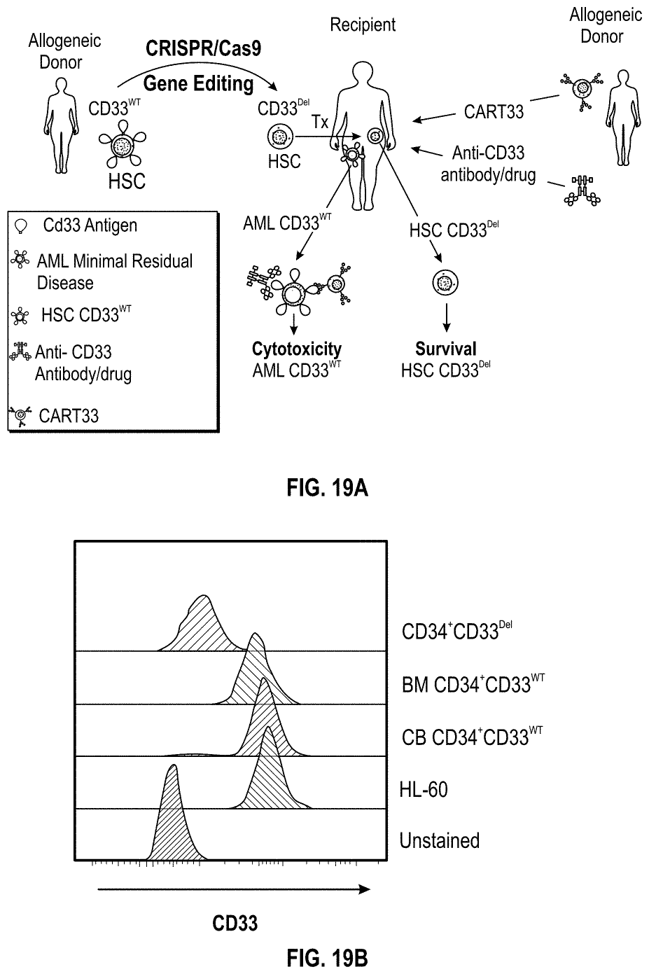

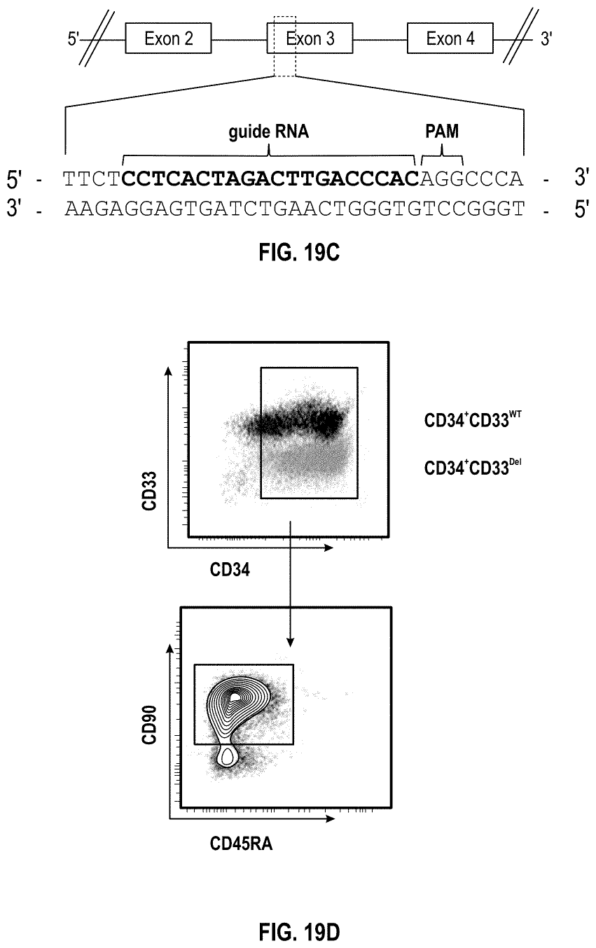

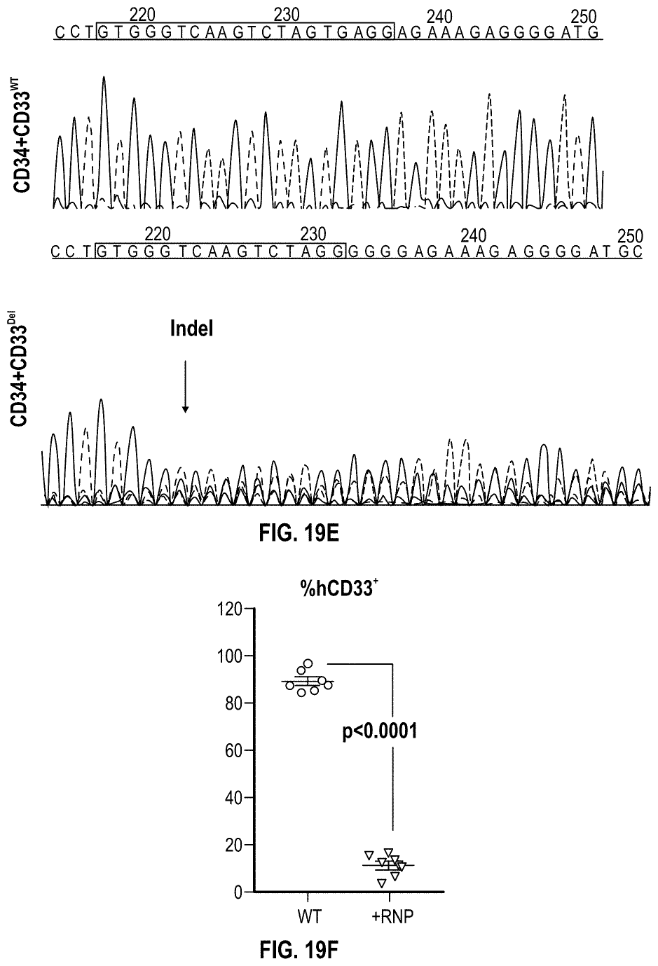

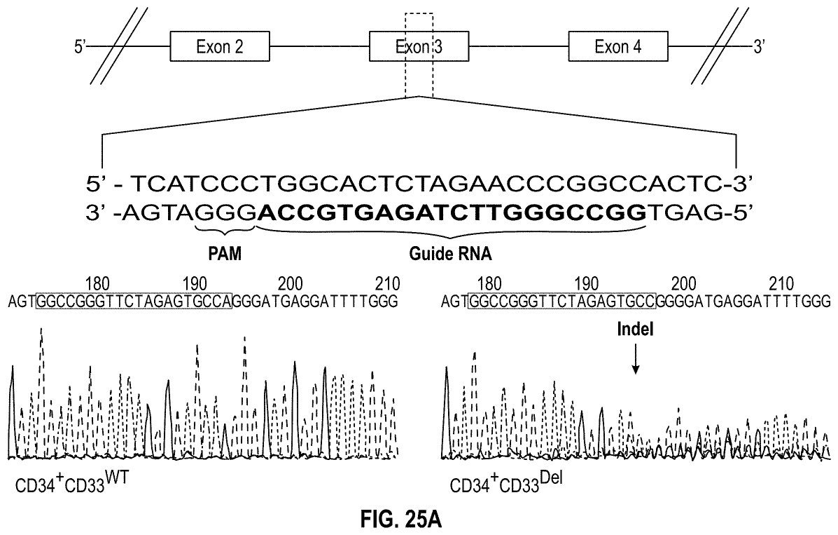

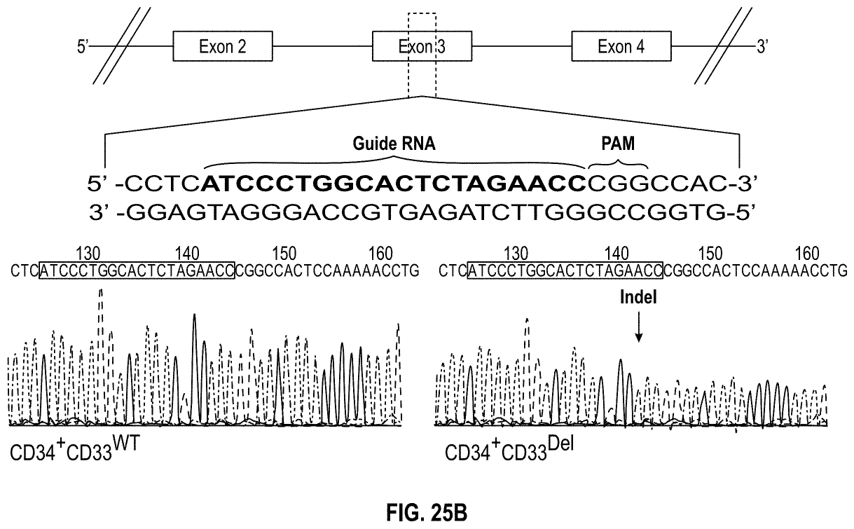

CD33 expressing HL-60 was used to model myeloid leukemia and primary CD34⁺ cells, either from cord blood (CB) or from adult bone marrow (BM), as the donor hematopoietic stem progenitor cell (HSPC). Surface expression of CD33 was confirmed in both HL-60 cells and in CD34⁺ cells using flow cytometry (FIG. 19B), as previously described (Taussig et al. Blood. 2005;106(13):4086-4092; Haubner et al. Leukemia. 2018;33(1):64-74; Wisniewski et al. Blood Cancer J. 2011;1(9):e36; Krupka et al. Blood. 2014;123(3):356-365). CRISPR/Cas9, a recently-developed versatile RNA-guided DNA editing technology (Haubner et al. Leukemia. 2018;33(1):64-74), was used to genetically edit genomic loci of CD33 to ablate its expression. Guides were designed to target exon 3 genomic loci (FIG. 19C, which shows the sgRNA 811 spacer sequence of SEQ ID NO: 58, FIG. 25A, which shows the spacer sequence of SEQ ID NO: 29, and FIG. 25B, which shows the sgRNA 846 spacer sequence of SEQ ID NO: 50) because exon 3 is common to all CD33 transcripts and has little to no similarity with Siglec family pseudogenes of which CD33 is a member. Plasmid, lentivirus, and ribonucleoprotein (RNP) based delivery systems were tested to check the efficiency of several guides in cell lines and primary cells and the RNP system in combination with chemically modified guides was found to be the most efficient in primary cells (FIGS. 19B and 19D). At the optimal conditions, the loss of CD33 expression was found in greater than 80% of CD34⁺HSPC, henceforth referred as CD33^{Del}, (FIGS. 19B and 19D) measured on a flow cytometer using the anti-CD33 clone HIM34, which recognizes an epitope located in the C2 domain common to all CD33 isoforms. This reduction in CD33 expression was accompanied by the presence of insertions/deletions (indels) at the expected cut site of Cas9 on DNA as measured by Sanger sequencing of DNA (FIG. 19E, bottom chromatogram). Two other sgRNAs, also targeting exon 3, were tested and displayed the same efficiency (FIGS. 25A and 25B). As expected, electroporation of Cas9 alone in the absence of sgRNA did not induce any indels at the target site (FIG. 19E, top chromatogram). CD33^{Del} cells retained high expression of CD34 and CD90 (FIG. 19D, bottom panel). Consistently high deletion efficiency was observed in several independent experiments (FIG. 19F), with on average 85% CD33 expression in CD33^{WT} cells down to less than 10% CD33 expression, 5 to 7 days post RNP electroporation. In addition, B-cells that lacked CD33 expression were used as a negative control to confirm loss of expression after Cas9 mediated deletion. The remaining 10% CD33 expression observed is likely from cells that are unedited (wildtype for both alleles) or partially edited (only one allele with indels).

CD34⁺ CD33^{Del} HSPCs Show Engraftment and Multilineage Differentiation In Vivo

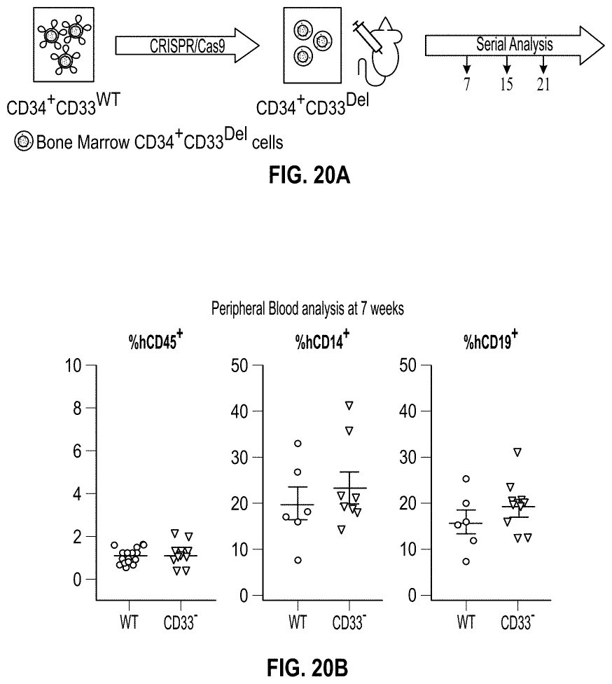

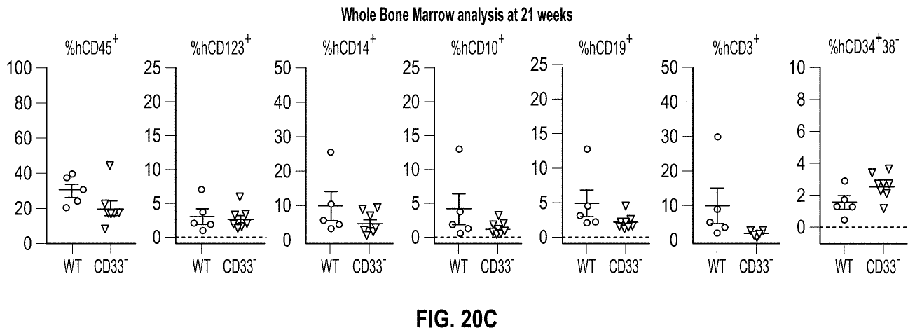

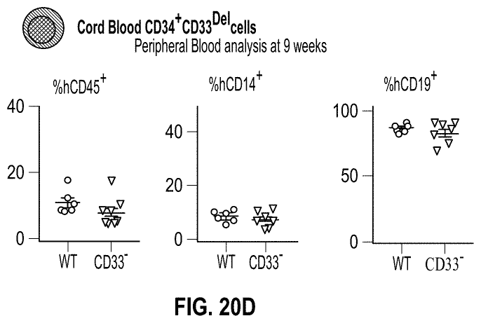

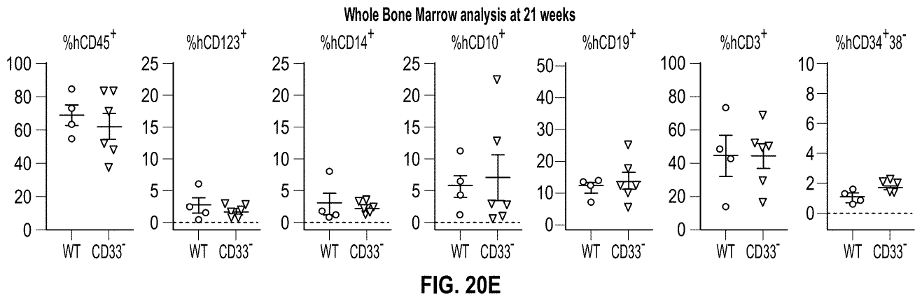

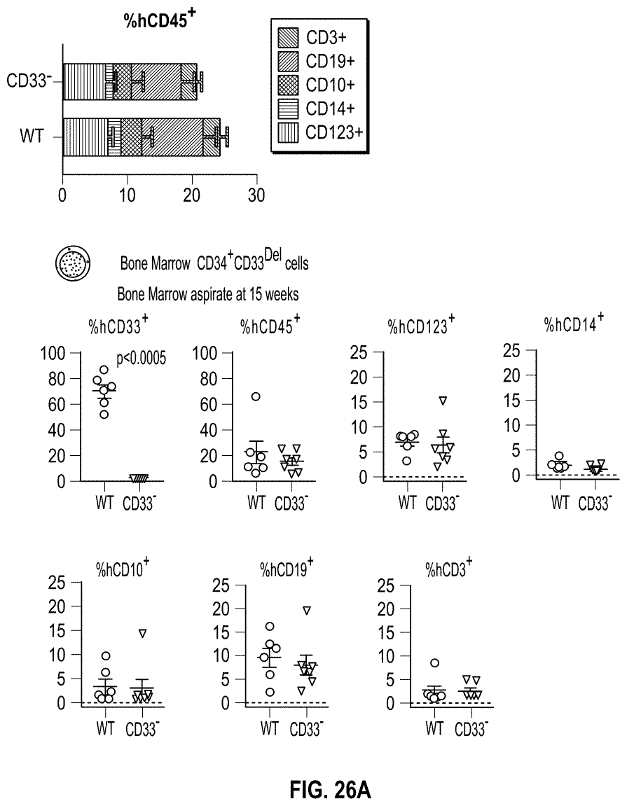

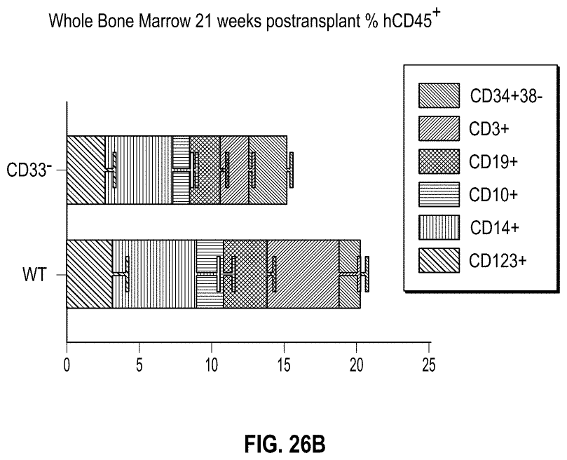

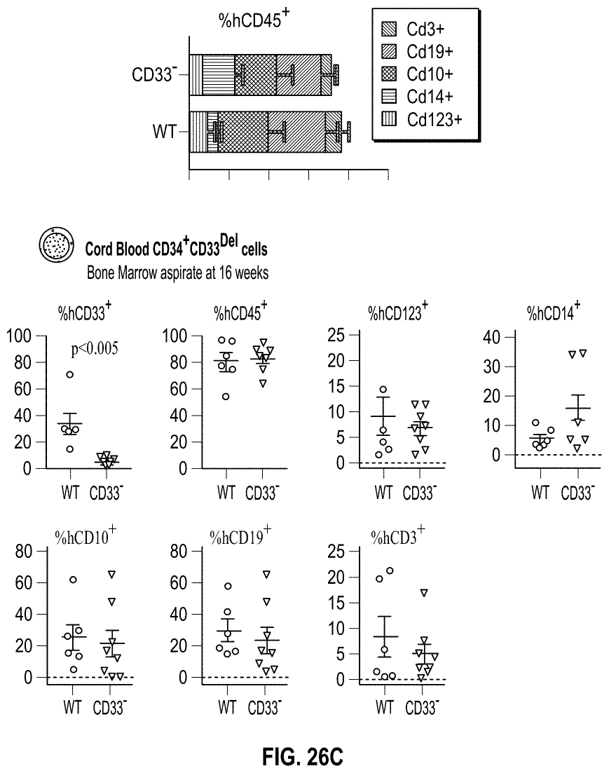

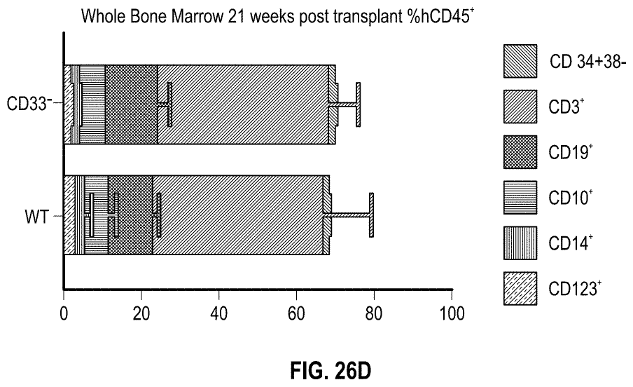

Since present approach can involve transplanting CD33 gene-edited stem cells (CD33^{Del}) as a platform for CAR-T targeting CD33 antigen (CART33) and ADC delivery (GO), it is important to test the ability of CD33^{Del} cells to engraft and contribute to myelopoiesis and lymphopoiesis. Both bone marrow and cord blood derived CD34⁺ cells were tested (FIG. 20). CD33^{Del} HSPCs were injected in sublethally irradiated NSG-SGM3 mice via a tail vein injection, and bone marrow and blood were analyzed (FIG. 20A). In mice injected with bone marrow-derived cells, peripheral blood analysis at 7 weeks post-transplant revealed the presence of human CD45⁺ cells and mature cells of myeloid (CD14⁺ cells) and lymphoid (CD19+ cells) origin (FIG. 20B). The analysis of bone marrow aspirate at 15 weeks (FIG. 26A) and whole bone marrow from sacked mice at 21 weeks (FIG. 20C), summarized in FIG. 26B, showed chimerism with a sustained contribution of human CD45⁺ cells over time. In all the tissues examined, there was multi-lineage engraftment with the presence of progenitors and mature cells of both myeloid (monocytes) and lymphoid (B-cells) origin. All cells remained CD33-negative (FIGS. 26A and 26C). No significant differences in multilineage engraftment of CD33^{Del} cells were observed when compared to wildtype cells.

In parallel, a similar strategy was followed with cord blood derived CD34⁺ cells and similar results were obtained. Multilineage engraftment was observed in peripheral blood at 9 weeks (FIG. 20D), in bone marrow aspirate at 16 weeks (FIG. 26C) and in whole bone marrow at 21 weeks post-transplant (FIG. 20E). The above results suggest that CD33 is not necessary for the engraftment of either the cord blood (CB) or bone marrow (BM) CD34⁺ cells or for the sustained repopulation of a complete human hematopoietic system in animal models.

### CD33^{Del} Cells are Proficient in Myeloid Differentiation and Function

Since the goal of the approach is its translation to the clinic, the functional capacity of myeloid CD33^{Del} cells was assessed in vitro and in vivo. First, the diversity of myeloid lineage was analyzed in CD34⁺CD33^{del} compared to CD34⁺CD33WT humanized mice and no noticeable difference was found among the myeloid subsets. The ability of monocyte differentiated CD34⁺CD33WT and CD34⁺CD33WT to phagocytose E coli bioparticles in vitro was tested. No significant difference was noticed. The LPS-induced cytokine production by monocytes/macrophages in NSGS mice transplanted with CD34⁺CD33WT or CD34⁺CD33del cells was also analyzed, and it was observed that plasma levels of TNFα, IL6 and IL8 after induction were comparable. Finally, to assess phagocytic function of CD33 deleted cells in vivo, the peritoneal cavity of humanized mice was analyzed, two hours after i.p. injection of E coli bioparticles. Flow cytometry analysis showed similar phagocytic uptake by the hCD45⁺hCD11b+hCD14−hCD16− subset in both CD34⁺CD33WT or CD34⁺CD33del humanized mice. All these findings show intact function of CD33Del myeloid cells.

### No Detectable Off-Target Mutagenesis or Loss of Functional p53 Pathways Observed in Gene-Edited Cells

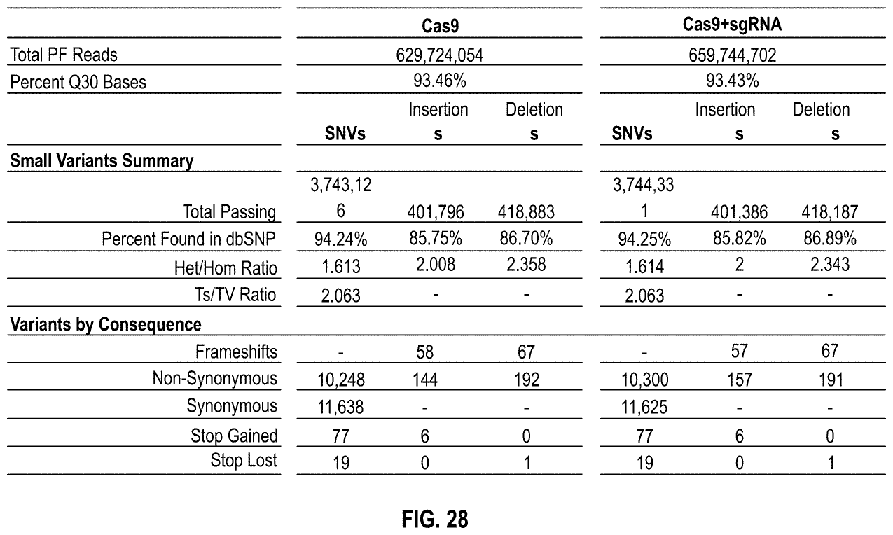

To evaluate whether the two guides used in this study introduce indels at off-target sites in HSP cells, indels were assessed in whole genome sequencing data of human cord blood CD34⁺ HSP cells electroporated with Cas9/sgRNA RNP complex (CD33^{Del}) compared with cells electroporated with Cas9 protein only (CD33^{WT}). Over 629 million passed filter reads were obtained with a base quality of over Q30 in over 93% of the reads (FIG. 28). The mean coverage depth was over 26×. To identify single-nucleotide variants (SNVs) and small indels, the reads were aligned to the human hg38 reference genome.

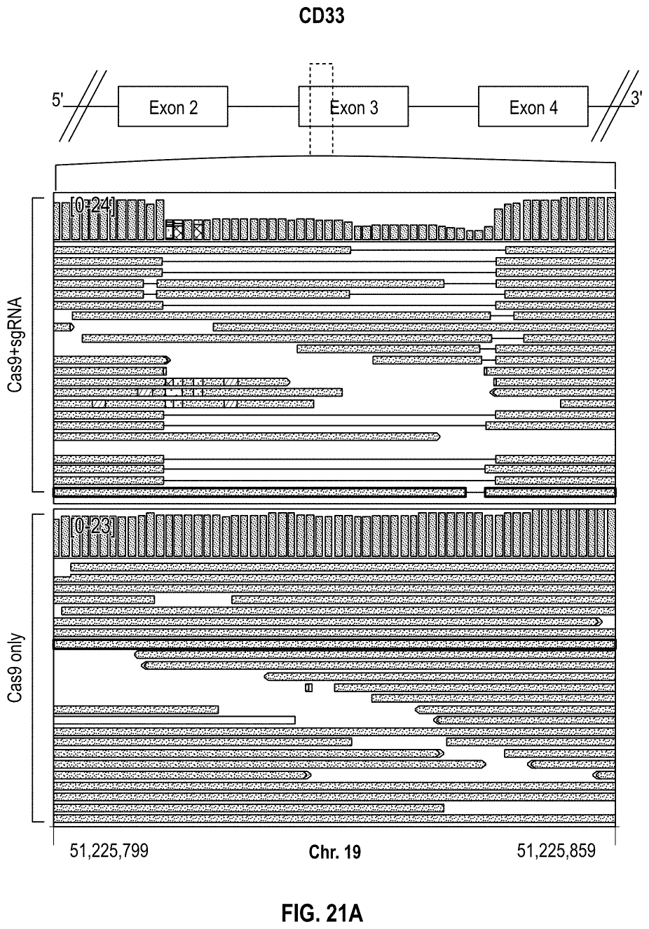

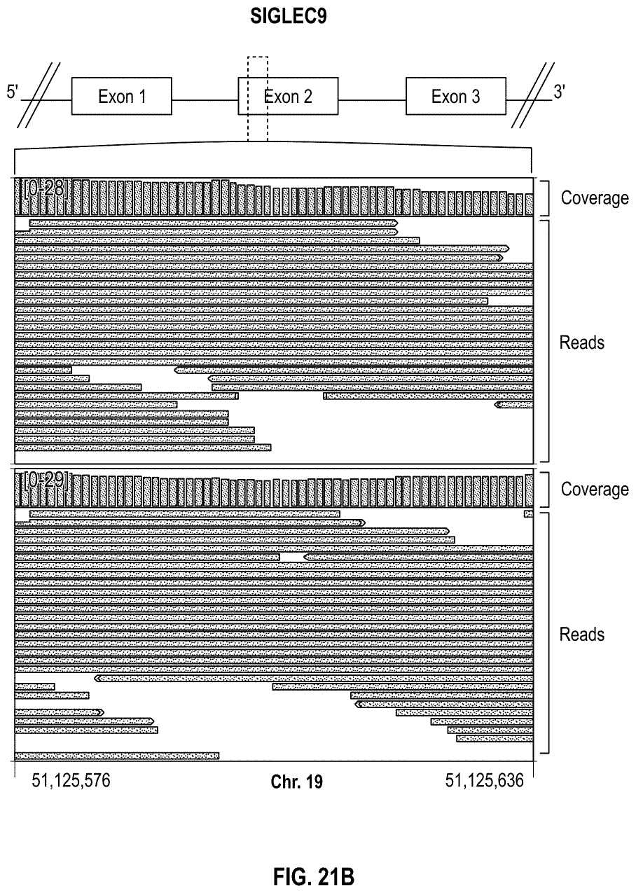

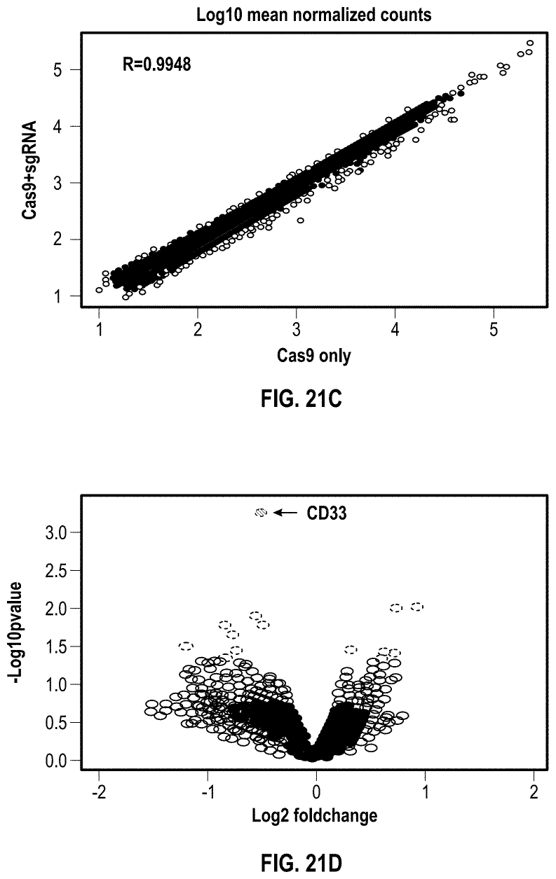

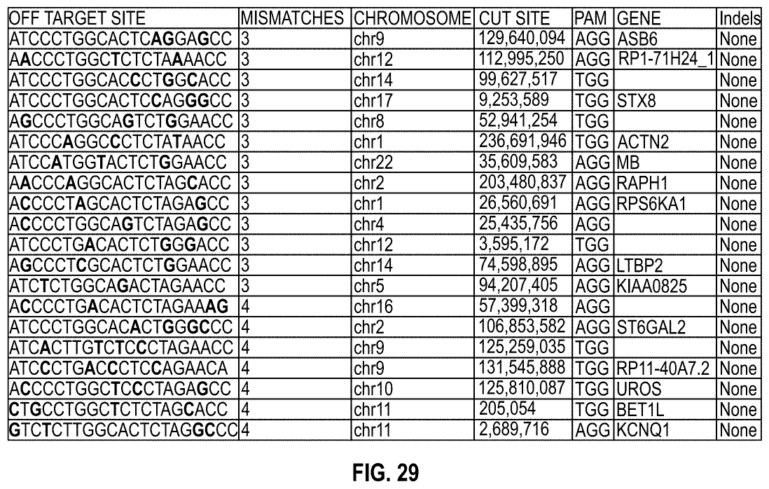

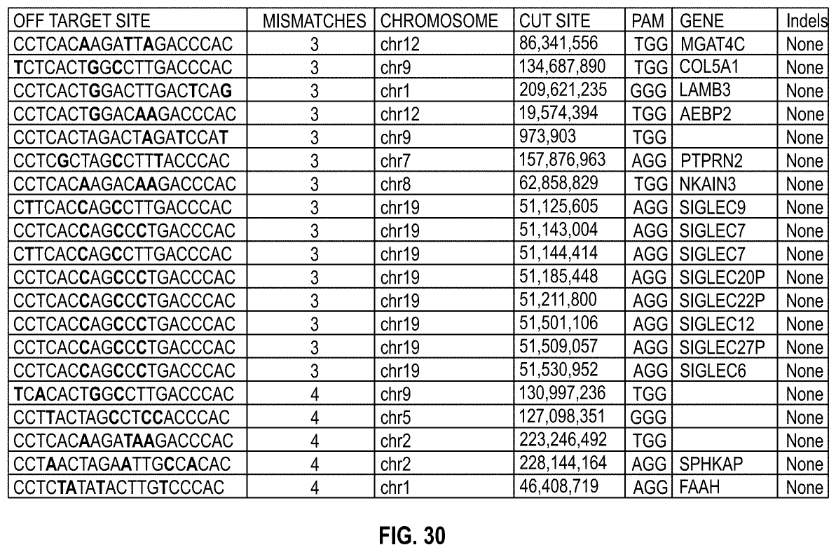

A summary of variants detected in both samples is presented in FIG. 28. A robust on-target activity was observed with indels in >90% reads aligning to the expected cut sites, chr19:51225811 and chr19:51225846 (FIG. 21A). Importantly, all indels were located within expected cut sites of the two sgRNA used. The few small indels that were observed outside of the targeted region in the entire CD33 locus were not unique to CD33^{Del} electroporated cells and were also present in cells electroporated with Cas9 only. The data was next examined for indels in predicted off-target sites that showed a high degree of similarity, with up to 4 mismatches with the sgRNAs used (FIGS. 21B, 29, and 30). Again, no indels were found in any the off-target loci examined (FIGS. 29 and 30). Indels were also examined in TP53 loci and none were found that were unique to CD33^{Del} cells.

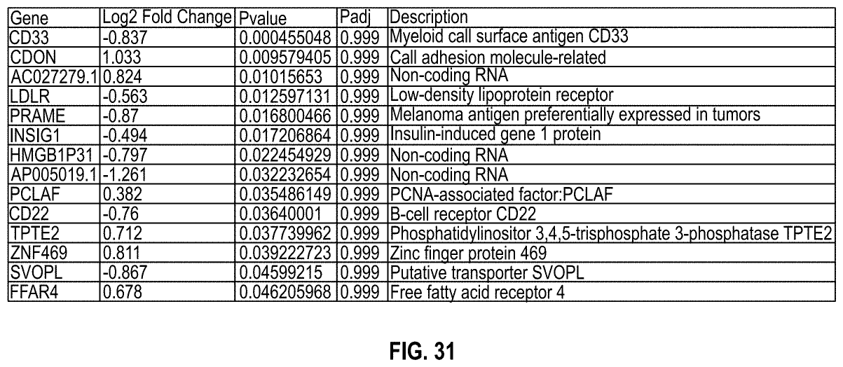

To assess whether the loss of CD33 expression causes changes in expression of other genes, gene expression profiles of CD33 deleted (n=5) and control (n=5) CD34⁺ cells obtained from four different donors were compared. The gene expression profile for each sample was obtained using RNA sequencing and comparison between groups was made using edgeR. Comparable gene expression profiles were observed between two groups with a Pearson correlation coefficient of 0.9948 (FIG. 21C) and no significant differences were observed based on p-adjusted value (FIG. 31). Fourteen genes were found to be significantly different based on p-value, and the most significant difference being the downregulation of CD33 in the CD33^{Del} samples compared to the controls (FIGS. 21D and 31). These results confirm that the absence of CD33 in CD34⁺ cells in vitro does not grossly affect downstream genes expression. Among the 13 genes that showed changed expression based on p-value, there was no enrichment for any one pathway or cellular process. Of note, gene expression signatures did not suggest that the TP53 pathway, or other DNA damage pathways, that could compromise HSC function or diminish their long-term potential, had been activated. It is therefore concluded that CD33 ablation in cord blood cells and adult HSCs using the gene editing technologies described herein does not appear to compromise their future function.

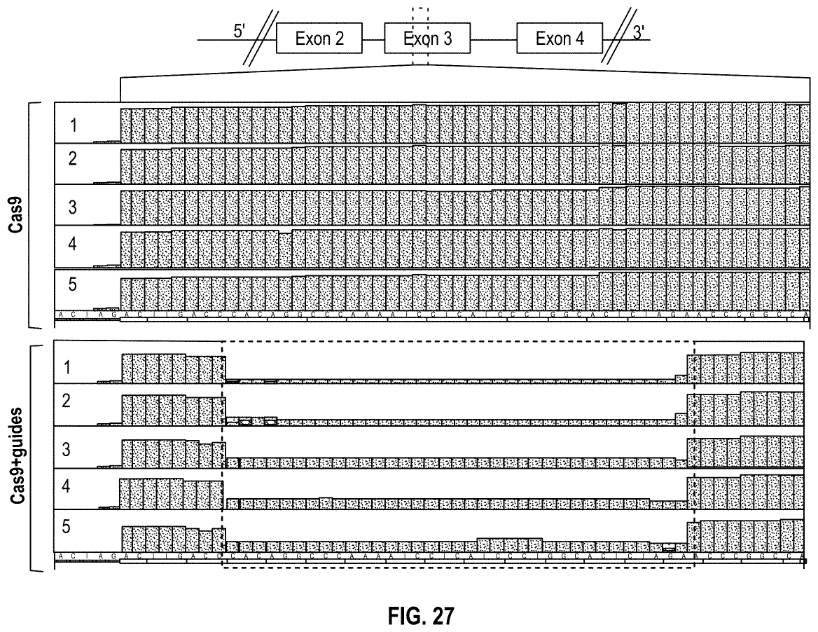

The data was also manually inspected for indels in reads mapping to exon 3 of CD33 and all coding exons of the TP53 transcript in RNAseq data using integrated genomic viewer (IGV). As expected, there were indels in >95% reads in CD33 exon 3 (FIG. 27). A few reads with indels were found in TP53, but they were within repeat sequences and were also present in control samples, suggesting sequencing artifacts. Together, these data suggest that CRISPR/Cas9 mediated genomic editing at CD33 locus using the guides used in this study results in no detectable off-target indels in the stem cells system.

### Expression of CD33 Specific CARs in T-Cells

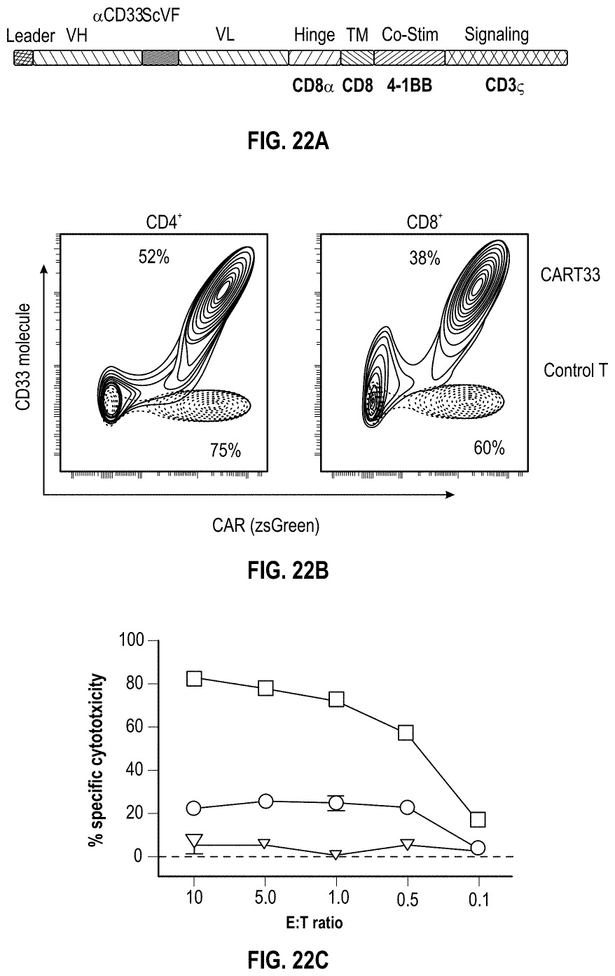

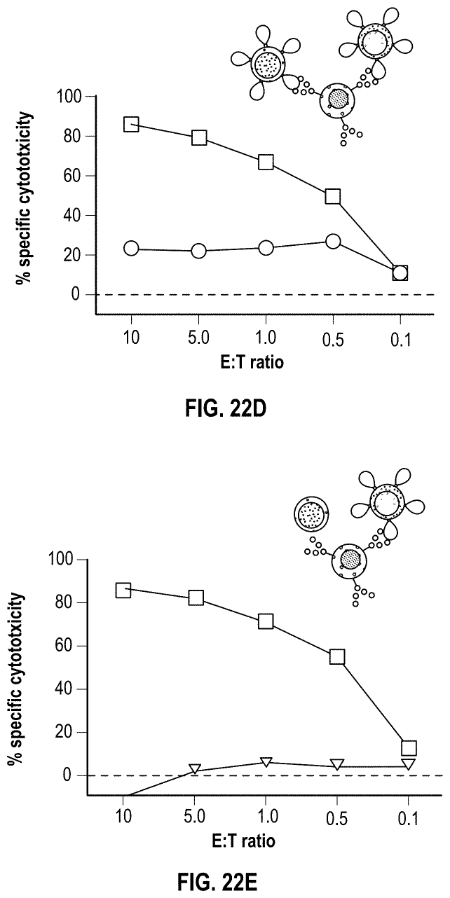

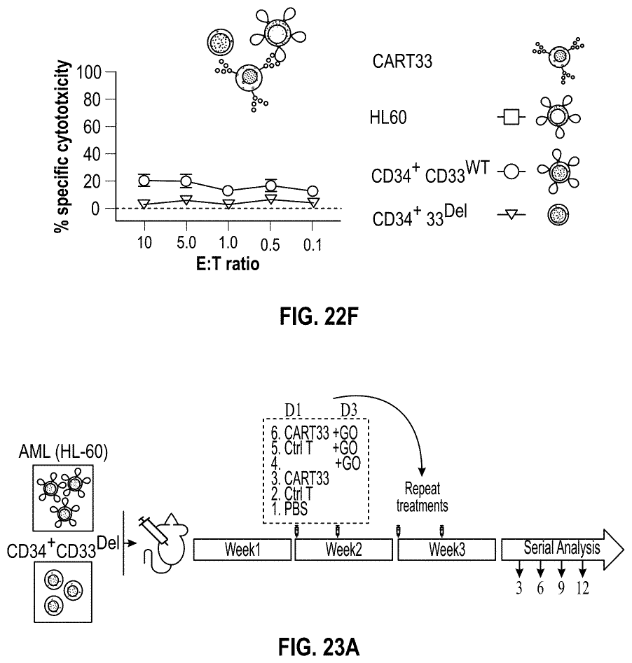

CARs are classified into different generations based on the number of co-stimulatory domains. A second-generation CAR has been designed, (FIG. 22A), including a single chain variable region of anti-CD33 (clone My96) paired with a CD28 transmembrane domain, a 4-1BB (CD137) costimulatory domain, and a CD3 zeta chain from CD3 TCR as an intracellular domain. The CAR cDNA was cloned into the pHIV-zsGreen lentiviral vector under the control of an EF1-α promoter, enabling bi-cistronic expression with ZS-green. Peripheral blood obtained from normal donors was fractionated to obtain PBMCs, and transduced with lentiviral particles carrying either vector only, or the CAR construct (FIG. 22B). The transduction efficiency of CD4 and CD8 cells within the PBMCs was not equal, as higher transduction of CD4 compared to CD8 cells was observed, and similar observations were also made in other studies (Blaeschke et al. Cancer Immunol Immunother. 2018;67(7):1053-1066). Given the unequal transduction of CD4 and CD8 cells in PBMCs, and in order to obtain a more defined composition of CD4 and CD8 cells, purified CD4 and CD8 cells were transduced separately, sorted based on GFP expression from a downstream IRES element, and mixed in an equimolar ratio. The CAR expression was confirmed by measuring the surface expression and binding of CAR to purified biotinylated CD33 protein conjugated to a streptavidin fluorochrome. A robust expression of the CAR and its binding with the CD33 molecule was observed (FIG. 22B).

### CAR Expressing T-Cells Show CD33-Dependent Cytotoxicity In Vitro

Cytotoxicity of CART33 cells was first evaluated over targets with variable CD33 expression. The high killing of CD33 myeloid leukemia cells HL-60 was confirmed, and lower killing of CD33^{WT} stem cells which express CD33 at a reduced level was observed. Notably, the absence of CD33 expression (due to Cas9/sgRNA mediated deletion) protected CD34⁺ cells from killing as no cytotoxicity of CART33 was observed when incubated with CD33^{Del} CD34⁺ cells. This first experiment (FIG. 22C) also confirmed a correlation between CART33 cytotoxicity level and CD33 expression level on target cells, i.e. that CART33 cytotoxicity is proportional to the expression level of CD33 on target cells.

A triple culture assay to assess CART33 cell killing when co-incubated with targets of variable CD33 expression was then designed. When CD33^{WT} HL-60 cells and CD34⁺CD33^{WT} cells were co-incubated with CART33, correlated cytotoxicity level of CART33 cells was observed over both cell types (FIG. 22D), but when CD33^{WT} HL-60 cells and CD34⁺CD33^{Del} cells were co-incubated with CART33 cells, only HL-60 cells were killed (FIG. 22E). Similarly, the co-incubation of all three cell types, CD34⁺CD33^{WT}, CD34⁺CD33^{Del}, and CART33 cells (FIG. 22F) displayed only killing of CD33^{WT} CD34⁺at a level proportional to its CD33 expression. Additionally, CART33 cytotoxicity was tested over another acute myeloid cell line, KG1, and similar CD33-dependent cytotoxicity of CART33 was found in vitro.

### Anti-CD33 Immunotherapy Show CD33-Dependent Leukemia Clearance in a Cell Line Derived Xenograft (CDX) Mouse Model

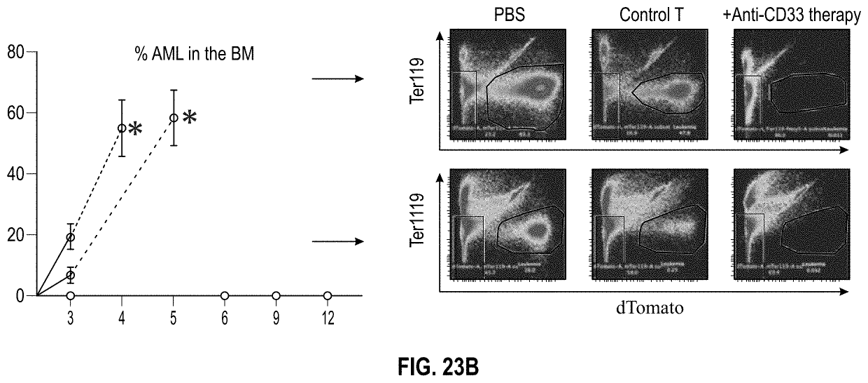

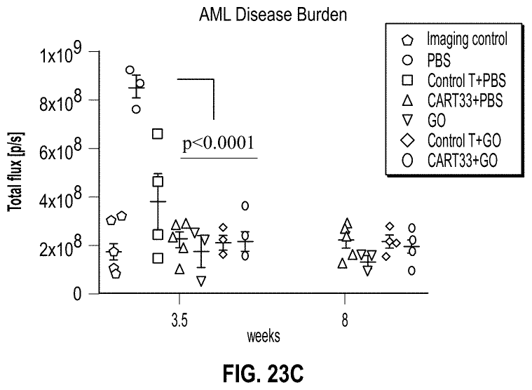

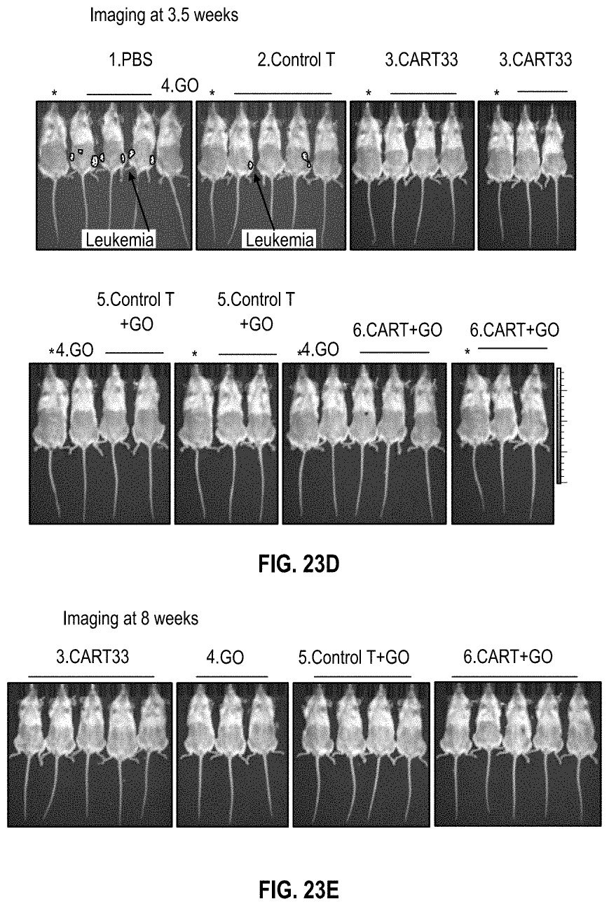

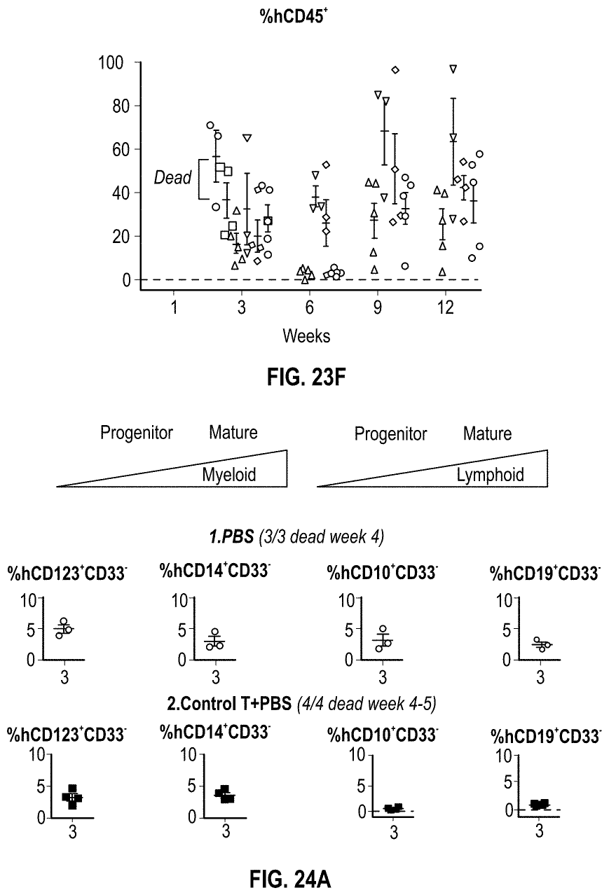

For in vivo experiments, a strategy was designed that to represent the human therapeutic setting in the context of minimal residual disease (FIG. 23A). In this model, leukemia was first initiated by injecting 500,000 HL-60 cells in sublethally irradiated mice. The mice were simultaneously injected with 500,000 CD33^{Del} CD34⁺ cells to mimic an AML relapsing model. Preliminary experiments suggested a one-week period was sufficient to enable homing and engraftment of AML cells, as 100 percent of mice to became leukemic after an additional 2 weeks. This recapitulates the post-remission bone marrow where AML cells may still remain although be clinically undetectable. One week after the co-injection of leukemia and CD34⁺ stem cells, mice were divided into various groups and treated with agents as described (FIG. 23A). Leukemia burden and CD34⁺ cell engraftment was monitored over time using imaging and flow cytometry (FIGS. 23B-23F). By week 3, high tumor burden was observed in the bone marrow aspirate of PBS and control CAR-T cell-treated mice (i.e. T cells transduced with the vector alone, lacking the CAR-T constructs), and by weeks 3 and 4 all the mice in these two groups died of their disease (FIG. 23A). Two control T treated mice had relatively low leukemia burden at 3 weeks but progressed to a very high leukemia burden in BM at death. On the contrary, over the course of 12 weeks, no CD33^{WT} leukemia cells were found in bone marrow aspirate (FIG. 23B) or on imaging at 3.5 weeks or 8 weeks (see FIGS. 23C-23E) of mice treated with CART33 cells, with the anti-CD33 ADC GO, or with a combination of GO and CART33. These results suggest that both CART33 and GO are potent agents against CD33-expressing leukemia in the present model.

### CD34⁺CD33′ HSPC Show Multilineage Engraftment and Differentiation in the Therapy Model

Simultaneously, mice were monitored for multilineage engraftment of CD34⁺CD33^{Del} cells in the therapy model described above. Engraftment was observed as demonstrated by the presence of human CD45⁺ cells that are CAR-T and CD33 negative in bone marrow aspirate of all the groups (FIG. 23F), suggesting that CD34⁺CD33^{Del} cells can also engraft in the present therapy model. Interestingly, a dip in the percentage of CD33^{Del} human CD45⁺ cells was observed at the week 6 time point relative to the initial point of week 3 in the CART33-treated groups (CART33+PBS and CART33+GO), but these levels were recovered to their initial levels by week 9 and maintained until the last point of week 12. The relatively low engraftment and the subsequent dip in the two groups of mice may reflect treatment-related stress (including the anti-leukemia response by CAR-T cells in the bone marrow) which was more pronounced in the CAR-T group and persisted longer than in the GO group. Alternatively, the use of different CD34⁺ and T cells donors might have triggered an allo-response that could explain the repopulation delay observed in mice injected with CART33 cells. Notably, this phenomenon was reversible as engraftment levels recovered over 8 weeks.

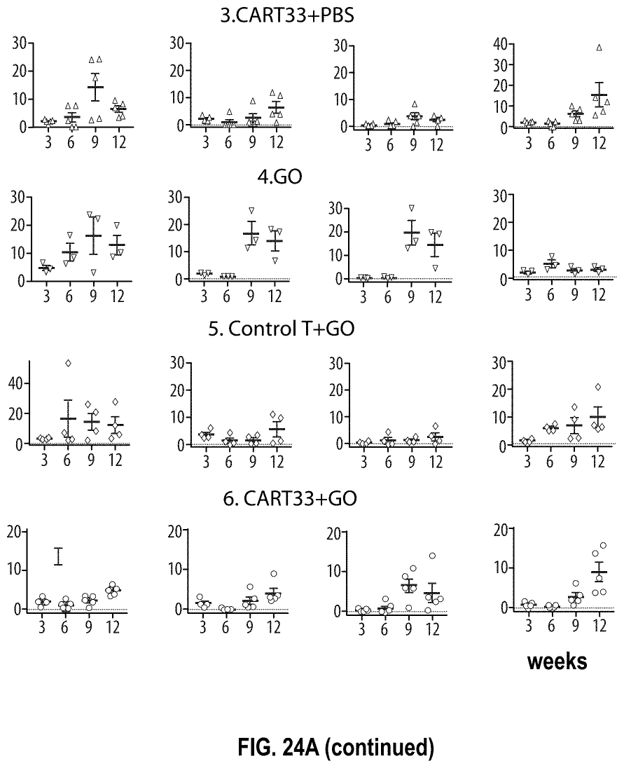

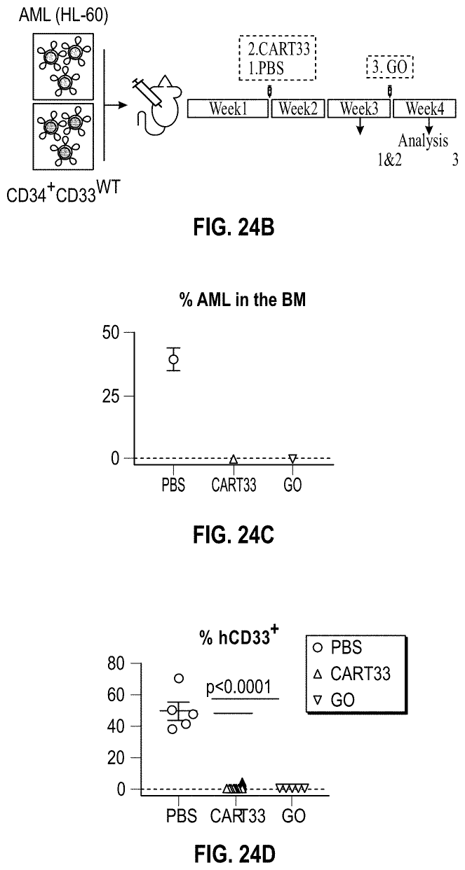

The multipotential nature of the engrafted cells was next investigated by analyzing myelopoiesis and lymphopoiesis (FIG. 24A). CD33 negative myeloid and lymphoid progenitors were found, as well as mature myeloid and lymphoid cells at all time points analyzed (FIG. 24A). While a full hematopoietic system repopulation was observed over time in all treated mice, a lymphoid repopulation delay was observed in mice injected with CAR-T cells. This could be the results of an allo response, as lymphoid progenitor cells are known to be more sensitive to an allo-specific effect.

Concomitantly, to demonstrate the specificity of CART33 and GO toward CD34⁺CD33^{WT} primary HSPCs, sublethally irradiated NSG-SGM3 mice were co-injected with 500,000 HL-60 cells and 500,000 CD34⁺CD33^{WT} cells (FIG. 24B). One week after, a group of mice was treated with CART33 cells and BM aspirated on week 3, the same day another group was injected with GO and their BM aspirate analyzed 4 days after. As observed in the therapy model, full leukemia clearance was confirmed following treatments (FIG. 24C). Additionally, full ablation of CD33^{WT} cells was observed (FIG. 24D). Therefore, CD33^{WT} cells remain sensitive to GO and CART33 therapies, while CD33 ablated cells are insensitive.

### Discussion

The success of any antigen-dependent immune therapy using agents like CAR-T or mABs is dependent on the presence of a unique antigen on the cancer cell surface and not on normal cells or other cells in the body. Unfortunately, such antigens are rare in cancers. One possible outcome was that by ablating LSA using genomic engineering methods in stem cells, it would be possible to generate stem/progenitor cells that are resistant to antigen-dependent immune therapy, thereby enabling maximal immunotherapy. After demonstrating that such antigen-depleted cells are functionally similar to the wildtype cells, they can be used to supplant the diseased cells. Careful selection of a lineage specific antigen that is dispensable to the normal function of that lineage is important to this approach. In an alternative approach, if an LSA is indispensable, instead of ablating the expression of the LSA completely, one can use gene-editing technology to modify the epitope recognized by the antigen-dependent immune therapy agent on LSA while maintaining the LSA function (termed “functionally redundant epitope switching”, or FRES).

In the present study, it was shown that combining stem cells lacking a lineage antigen, CD33, with allogeneic engineered T cells or an ADC, leukemia ablation and full hematopoietic repopulation can be enabled. Acute myeloid leukemia, a disease with unmet need in the area of therapy, was used, and it was demonstrated that such an approach is feasible. Since CD33 is an LSA and targeting of CD33 in AML, using either CAR-T or CD33 mABs, results in severe myelosuppression and lympho-depletion due to the elimination of stem/progenitor cells as well as cells of myeloid lineage, the proposed approach for treating AML is to rebuild the hematopoietic system with cells lacking CD33. A CRISPR-based approach was used to disrupt CD33 expression in donor stem cells, either cord blood or bone marrow CD34⁺ cells, to render them “resistant” to CAR-T cell attack.

Recently, two groups made similar observations and independently reported the approach described in this study (Kim et al. Cell. 2018;173(6):1439-1453 e1419; Humbert et al. Leukemia. 2019;33(3):762-808). The present data strengthen the observations made by these studies and also add novel insights using complementary approaches. Unlike the Kim et al. study, in which the mice were first injected with CD33 gene-edited CD34⁺ cells to allow for complete engraftment before the leukemia introduction and treatment, the present approach more closely mimics the situation of AML relapse with minimal residual disease since leukemia cells and gene-edited stem cells are co-injected, followed by CAR-T or ADC therapy. Furthermore, by stringently selecting CD33 guide RNAs with high on-target and low off-target activity, extremely efficient ablation of CD33 expression was observed in HSCs with no off-target indels observed in other genes, thus enabling confidence in the safety of this approach in human studies (Kim et al. Cell. 2018;173(6):1439-1453 e1419). Indeed, no indels were found within any of the Siglec family of genes and pseudogenes examined. Kim et al. observed off-target activity in SIGLEC22P, including a deletion of 14 kb fragment, most likely due to 100% homology with SIGLEC22P of the sgRNA designed for CD33 (Kim et al. Cell. 2018;173(6):1439-1453 e1419). The choice of location within exon 3, and the confirmation of the absence of homology of the chosen sgRNA with other genes, may have enabled the specificity of CD33 only ablation. The absence of indels or other genomic rearrangements in TP53 gene (as analyzed using whole genome sequencing), and the absence of any deregulated genes related to the p53 pathway or p53 itself, suggest that the approach developed herein might provide efficient genome-editing with high specificity without compromising HSC function. Finally, the use of Cas9 RNP (which is only present transiently in the HSCs during their ex vivo manipulation), as opposed to virally-mediated expression of Cas9 that is constitutive and continues in the HSCs in vivo, avoids future issues with preexisting immunity against Cas9 which is found to be present in >50% of the population (Charlesworth et al. Nat Med. 2019;25(2):249-254; Crudele et al. Nat Commun 2018;9(1):3497).

Furthermore, in contrast to Kim et al., the present approach involves allo-BMT with CD33 edited HSCs (from cord blood or adult bone marrow) followed by ADC treatment or CAR-T treatment with T cells derived from the allogeneic donor. This approach is more practical in the clinical setting. Patients with hematologic malignancies who have been heavily pre-treated with cytotoxic chemotherapies often produce poor autologous T cells yields, limiting the efficiency and effectiveness of autologous CAR-T. This problem is circumvented by using allo-BMT and allo-T cells, where yield and quality is not an issue. More importantly, by using ADC (rather than CAR-T) to target the disease, it is shown that humoral therapy can act in concert with, or as an alternative to, CAR-T cells, further expanding the approach to anti-leukemia therapy using humoral approaches.

It is also noted that the GO drug (comprised of the anti-CD33 antibody clone P67.6) recognizes an epitope located in exon 2. Two isoforms of CD33 are found in humans. The more common isoform is the full-length protein including exon 2 that is sensitive to GO; the less common is an isoform that lacks exon 2. Around 30% of the population carries a homozygote single nucleotide polymorphism (SNP, T/T) resulting in the exclusive expression of the less common CD33 variant that lacks exon 2. This population could also be considered a potential pool of HSCT donors in combination with the targeted CD33 immunotherapy described in this work, thereby eliminating the need for Cas-9 directed ablation. However, this might limit the donor pool drastically, making the approach practically unfeasible in human studies. In this regard, Humbert et al. (Humbert et al. Leukemia. 2019;33(3):762-808), used a CRISPR/Cas9 approach to target flanking introns using two different sgRNAs to delete exon 2. It is unclear whether selectively removing V-domain has any advantage over disruption of the entire CD33, as engraftment or functional defects were not observed in either mice or monkeys (Rhesus macaques) by removing entire CD33 (Kim et al. Cell. 2018;173(6):1439-1453 e1419). Additionally, the use of multiple guides multiplies potential off-targets and the efficiency of the guides will be likely limited by the least efficient guide in the pool.

In the present study, the deletion of CD33 in human CD34⁺ BM and CB did not result in any noticeable side effects. More than 21 weeks after transplantation, NSG-SGM3 mice have not presented any abnormal phenotypes. The absence of observable CD33 deletion linked phenotype might be explained by functional redundancy or compensation among Siglecs members.

Despite an increasing number of clinical trials involving modified immune cells, few have resulted in improved outcomes for patients, mainly because of the on-target/off-tissue toxicity on normal tissues. While CAR-T is a recent approach whose long-term effects are less well established, the use of mABs and ADCs is routine in cancer care and generally safe. It is shown that combining CART cells and/or ADC such as GO with engineered stem cells protects normal tissues from on-target/off-tissue toxicity and can lead to full remission and full hematopoietic reconstitution in an animal model. Despite proven benefits, virtually all GO treated patients experience substantial depletion of normal myeloid lineage cells, which can lead to potentially lethal febrile neutropenias and abnormal bleeding problems due to GO induced myelosuppression (Amadori et al. J Clin Oncol. 2016;34(9):972-979). These severe adverse events limit the use of GO to brief exposures during induction chemotherapy and essentially prohibit its chronic use. The research also suggests that the combination of lower GO doses with or without CART33 infusion could be a fundamentally new approach to treat AML patients. Finally, the recent re-approval of GO, and the resurgence of current clinical trials of novel CD33 directed reagents (including new anti-CD33 CAR-T, anti-CD33 ADCs and CD33 bi-specific T cell engagers or BiTEs) render this approach transferable to the clinic in the near future.

An easily usable pipeline has also been designed to test new potential targets that share the properties that make CD33 an attractable target i.e. a functionally redundant lineage marker that is strictly expressed by hematopoietic cells and also expressed by the cancer cells (e.g., CD123, CLL-1 or CD244). This antigen is rendered “cancer specific” by CRISPR mediated ablation of the antigen from HSCs (Haubner et al. Leukemia. 2018;33(1):64-74). The strategy could also be replicated in solid tumors where a functional organoid might be generated from embryonic or induced pluripotent stem cells that are edited to ablate expression, or where the primary organ has already been removed (i.e. to target a normal prostate lineage antigen in a patient after radical prostatectomy). Finally, the possibility of “epitope modification” of a tissue specific antigen using DNA base-editing methods is proposed. “Epitope modification” might allow a protein to retain its function, but switch a small antigenic determinant. In this strategy, the stem cells retain a functional protein, but no longer possess the binding site for the immune therapy, while the cancer cells, carrying the unmodified protein, remain uniquely sensitive to immune therapies.

### EXAMPLE 6: Gentuzumab Ozogamicin (GO)-Targeted Immunotherapy Cleared Primary Acute Myeloid Leukemia (AML) and Rescued CD33^{Del} Cells

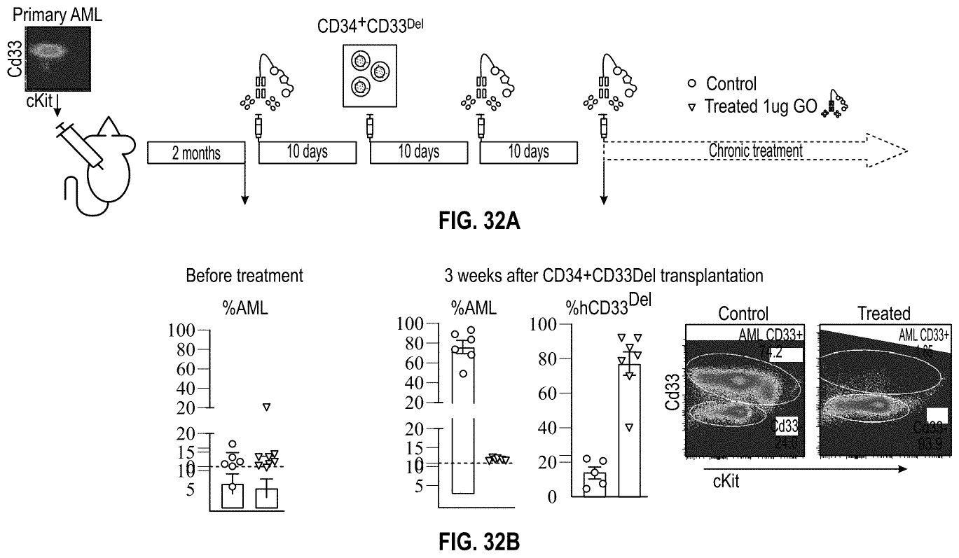

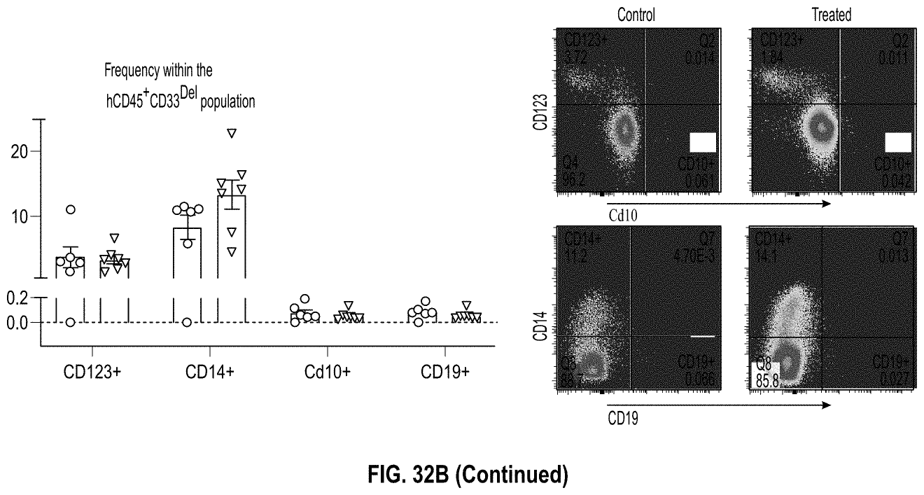

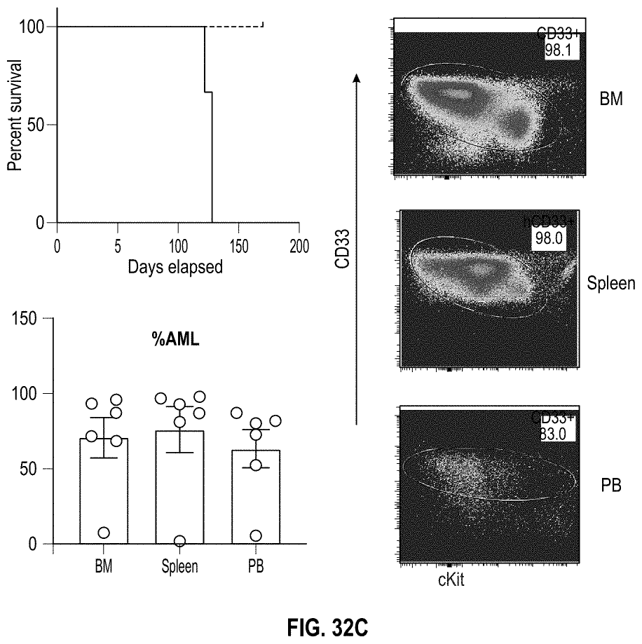

CD34⁺CD33^{Del} cells were generated by contacting a population of CD34+ cells with gRNAs sgRNA 811 (having a guide region of 5′ CCUCACUAGACUUGACCCAC 3′; SEQ ID NO: 70) and sgRNA 846 (having a guide region of 5′ AUCCCUGGCACUCUAGAACC 3′; SEQ ID NO: 67). In order to assess CD34⁺CD33^{Del} cells engraftment and hematopoietic repopulation upon GO treatment, 0.5 ×10⁶ primary human AML cells were injected into NSG-SGM3 (NSGS) mice intravenously via tail vein on day one. Two months post AML cells injection, the AML burden (minimal residual disease) was assessed by flow cytometry of bone marrow (BM) aspirate. The cohort was then divided in two comparable groups (FIG. 32A graph: Before Treatment): the control group (circles) in which mice will not receive treatment and the treated group (triangles) in which mice will receive chronic dose of GO. Leukemia cells were gated on Ter119⁻H2kd/Ly5⁻hCD45⁺¹¹CD33⁺cKit using the following antibodies: Ter119 Pecy5, Ly5/H2kd BV711, hCD45BV510, hCD33 APC, cKit BV650. Once minimal residual disease was assessed, mice from the treated group received a first injection of 1 μg of GO. Ten days after the first GO injection, both mice groups (untreated and treated) were transplanted with 0.5 ×10⁶ CD34⁺CD33^{Del} cells. Ten days after CD34⁺CD33^{Del} cells transplantation, the treated group started to receive chronic dose of GO (1 μgr GO injected every 10 days). Three weeks after CD34⁺CD33^{Del} transplantation, BM aspirates of both mice groups were analyzed by flow cytometry for AML burden and CD34⁺CD33^{Del} cells engraftment assessment. In control mice, AML levels were greater than 70%, while in treated mice AML levels were below 5%. In control mice, the percentage of hCD33^{Del} cells was below 20%, while in treated mice the percentage of hCD33^{Del} cells was above 70%, indicating successful engraftment. (FIG. 32B, top graph). Next, hematopoietic repopulation ability of the injected CD34⁺CD33^{Del} cells was evaluated by analyzing myeloid/lymphoid progenitor and mature cells by flow cytometry as shown in FIG. 32B, continued). Levels of CD123+ cells, CD14+ cells, CD10+ cells, and CD19+ cells within the hCD45⁺CD33^{Del} population (cells were gated on Ter119⁻, Ly5⁻/H2kd⁻, hCD45⁺, hCD33⁻) were not significantly different between control and treated cohorts. As shown in FIG. 32C, top left panel, no control mice survived 150 days, while all treated mice survived at least 150 days. The AML burden was comparable among the BM, spleen and peripheral blood of control mice at the time of death (lower left panel). Moreover, CD33 expression was most notable in the BM and spleen as shown by gated CD33+ cells from each respective organ or tissue (right panel).

### EXAMPLE 7: Generation of CD34⁺CD33^{Del}CLL1^{Del} Cells

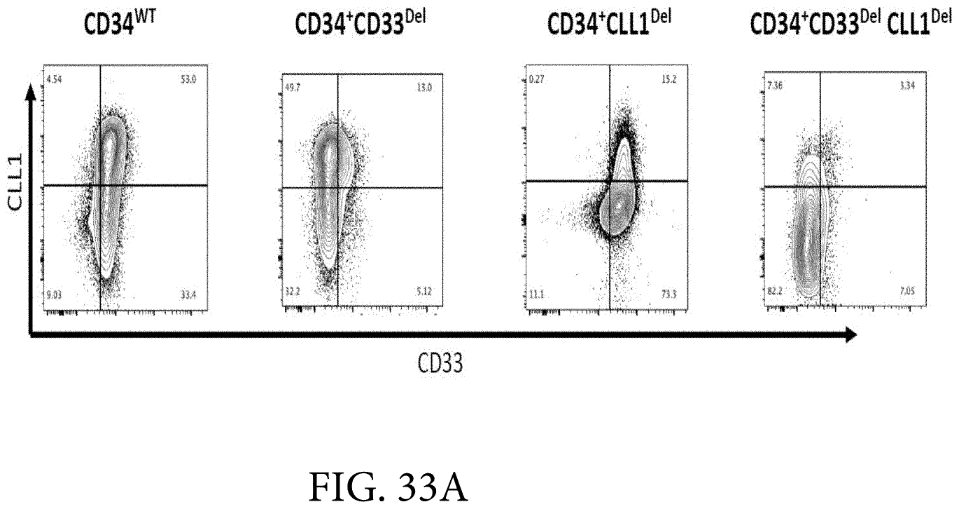

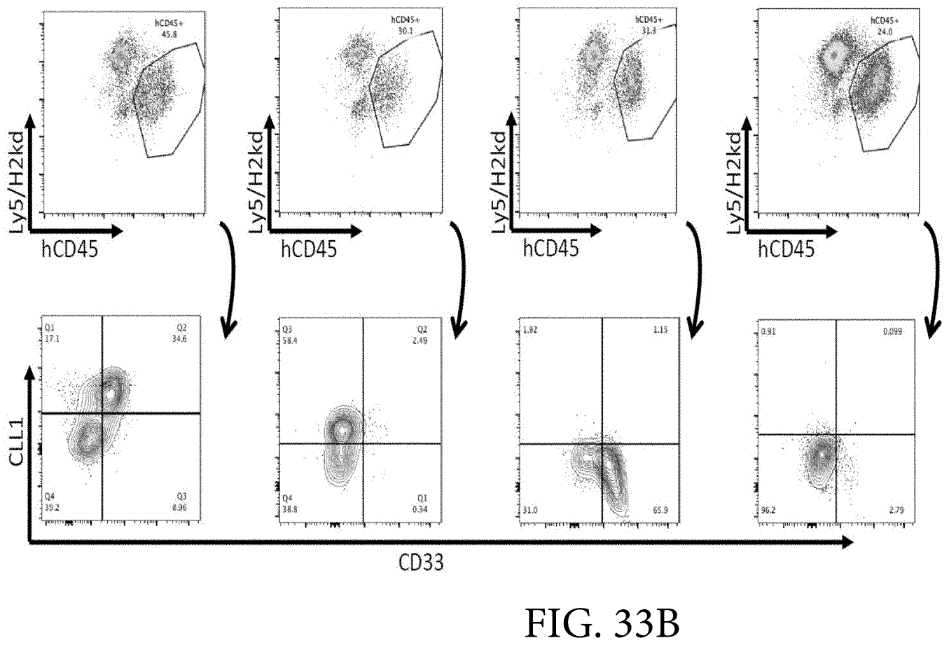

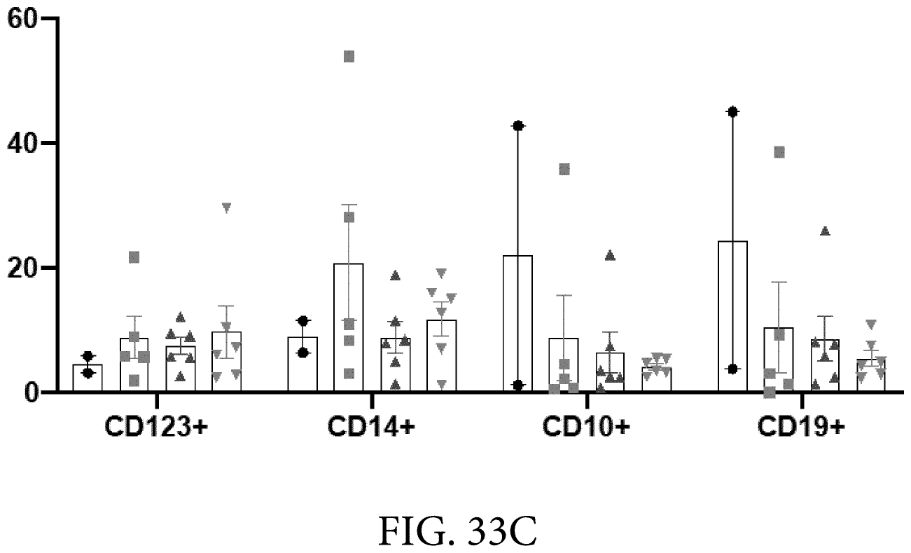

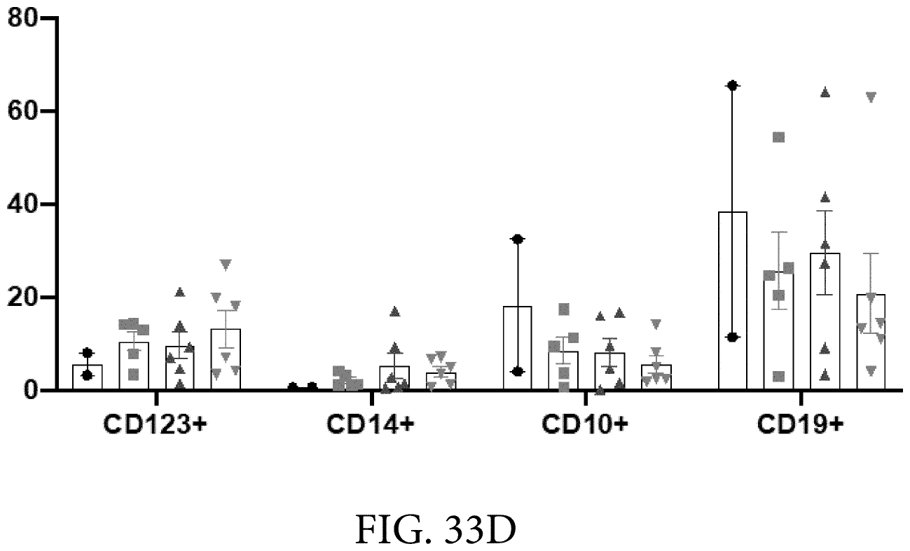

CD34⁺CD33^{Del}CLL1^{Del} double deletion cells were generated by transfecting CD34⁺ cells with different combinations of gRNAs, including: sgRNA 811 (having a spacer sequence of 5′ CCUCACUAGACUUGACCCAC 3′ against CD33; SEQ ID NO: 70), sgRNA 846 (having a spacer sequence 5′ AUCCCUGGCACUCUAGAACC 3′ against CD33; SEQ ID NO: 67), a CLL-1 gRNA having a spacer sequence of 5′ GUUGUAGAGAAAUAUUUCUC 3′ (SEQ ID NO: 115) and a second CLL-1 gRNA having a guide region 5′ GGAGAGGUUCCUGAUCUUGU 3′ (SEQ ID NO: 116). On day 1, cells were transfected with the sgRNAs using nucleofection in order to obtain after CRISPR/Cas9-mediated ablation of the target genes. Four days post-transfection, the expression of CD33 and CLL1 was assessed by flow cytometry. On day 5, CD34^{+WT}, CD34⁺CD33^{Del}, CD34⁺CLL1^{Del} or CD34⁺CD33^{Del}CLL1^{Del} cells were intravenously injected into NSGS mice. As shown in FIG. 33A, CD33 and/or CLL1 levels were depleted successfully individually and in combination using these gRNAs. Mutations at the desired loci were confirmed by Sanger sequencing (data not shown).

Four weeks post-injection, bone marrow (BM) aspirates of injected mice were analyzed by flow cytometry to determine the presence of CD33 and/or CLL1 in CD34⁺ cells gated on Ter119⁻, Ly5⁻/H2kd⁻, hCD45⁺(FIG. 33B). The single and double deletion cells were successfully detected in the bone marrow samples. Furthermore, CD123+, CD14+, CD10+, and CD19+ cells were detected in the hCD45+population in both whole bone marrow samples (FIG. 33C) and spleen samples (FIG. 33D). This analysis shows that depletion of CD33 and/or CLL1 did not impair hematopoietic multilineage engraftment.

| | | | | SEQ |
|---|---|---|---|---|
| | | | | ID |
| Target | Guide sequence | Cut site | Exon | NO: |
| CD33 | CCUCACUAGACUUGACCCAC | 51,225,811 | 3 | 70 |
| CD33 | AUCCCUGGCACUCUAGAACC | 51,225,846 | 3 | 67 |
| CLL1 | GUUGUAGAGAAAUAUUUCUC | 9,979,406 | 3 | 115 |
| CLL1 | GGAGAGGUUCCUGAUCUUGU | 9,979,450 | 3 | 116 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A genetically engineered hematopoietic stem or progenitor cell, which comprises a genetic mutation in exon 3 of an endogenous CD33 gene, wherein the genetic mutation is at a site targeted by a gRNA which comprises the nucleotide sequence of CCUCACUAGACUUGACCCAC (SEQ ID NO: 70), and wherein the genetic mutation causes a reduced expression level of CD33 as compared with a wild-type counterpart cell.

2. The genetically engineered hematopoietic stem or progenitor cell of claim 1, which expresses less than 30% of the CD33 expressed by the wild-type counterpart.

3. The genetically engineered hematopoietic stem or progenitor cell of claim 1, which expresses less than 20% of the CD33 expressed by the wild-type counterpart.

4. The genetically engineered hematopoietic stem or progenitor cell of claim 1, which expresses less than 10% of the CD33 expressed by the wild-type counterpart.

5. The genetically engineered hematopoietic stem or progenitor cell of claim 1, which expresses less than 5% of the CD33 expressed by the wild-type counterpart.

6. The genetically engineered hematopoietic stem or progenitor cell of claim 2, which does not express CD33.

7. The genetically engineered hematopoietic stem or progenitor cell of claim 1, wherein the genetic mutation is a substitution, an insertion, or a deletion, or a combination thereof.

8. The genetically engineered hematopoietic stem or progenitor cell of claim 7, wherein the deletion is 1, 2, 4, 5, 7, 8, 10, 11, 13, 14, 16, or 17 nucleotides in length.

9. The genetically engineered hematopoietic stem or progenitor cell of claim 1, wherein the genetic mutation results in a frameshift.

10. The genetically engineered hematopoietic stem or progenitor cell of claim 1, which is CD34+.

11. The genetically engineered hematopoietic stem or progenitor cell of claim 1, which is from bone marrow cells or peripheral blood mononuclear cells of a subject.

12. The genetically engineered hematopoietic stem or progenitor cell of claim 11, wherein the subject is a human patient having a hematopoietic malignancy.

13. The genetically engineered hematopoietic stem or progenitor cell of claim 11, wherein the subject is a healthy human donor.

14. A cell population, comprising a plurality of the genetically engineered hematopoietic stem or progenitor cells of claim 1.

15. The cell population of claim 14, wherein at least 50% of cells in the population comprise genetic mutations in two copies of CD33.

16. The cell population of claim 14, wherein at least 60% of cells in the population comprise genetic mutations in two copies of CD33.

17. The cell population of claim 14, wherein at least 70% of cells in the population comprise genetic mutations in two copies of CD33.

18. The cell population of claim 14, wherein at least 80% of cells in the population comprise genetic mutations in two copies of CD33.

19. The cell population of claim 14, wherein at least 90% of cells in the population comprise genetic mutations in two copies of CD33.

20. The cell population of claim 14, wherein at least 95% of cells in the population comprise genetic mutations in two copies of CD33.

21. The cell population of claim 14, wherein at least 50%, 60%, 70%, 80%, 85%, 90%, or 95% of cells in the population comprise genetic mutations in two copies of CD33.


SEQUENCE LISTING (119 sequences)

SEQ ID NO: 1 - DNA, 339 bp, Artificial Sequence
  Description of Artificial Sequence: Synthetic polynucleotide
gagatcgtgctgacccagagccccggcagcctggccgtgagccccggcgagagggtgaccatgagctgcaagagcagccagagcgtgttcttcagcagcagccagaagaactacctggcctggtaccagcagatccccggccagagccccaggctgctgatctactgggccagcaccagggagagcggcgtgcccgacaggttcaccggcagcggcagcggcaccgacttcaccctgaccatcagcagcgtgcagcccgaggacctggccatctactactgccaccagtacctgagcagcaggaccttcggccagggcaccaagctggagatcaagagg

SEQ ID NO: 2 - DNA, 354 bp, Artificial Sequence
  Description of Artificial Sequence: Synthetic polynucleotide
caggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttccagggcaaggccaccctgaccgccgacaagagcagcaccaccgcctacatgcagctgagcagcctgaccagcgaggacagcgccgtgtactactgcgccagggaggtgaggctgaggtacttcgacgtgtggggccagggcaccaccgtgaccgtgagcagc

SEQ ID NO: 3 - DNA, 657 bp, Artificial Sequence
  Description of Artificial Sequence: Synthetic polynucleotide
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - DNA, 603 bp, Artificial Sequence
  Description of Artificial Sequence: Synthetic polynucleotide
ctatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - DNA, 627 bp, Artificial Sequence
  Description of Artificial Sequence: Synthetic polynucleotide
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - protein, 107 aa, Artificial Sequence
  Description of Artificial Sequence: Synthetic polypeptide
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 89 aa, Artificial Sequence
  Description of Artificial Sequence: Synthetic polypeptide
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 8 - protein, 97 aa, Artificial Sequence
  Description of Artificial Sequence: Synthetic polypeptide
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 9 - DNA, 23 bp, Artificial Sequence
  Description of Artificial Sequence: Synthetic oligonucleotide
ccaaagagtccggggatacttgg

SEQ ID NO: 10 - DNA, 23 bp, Artificial Sequence
  Description of Artificial Sequence: Synthetic oligonucleotide
gttgagttttgcattggcggcgg

SEQ ID NO: 11 - DNA, 23 bp, Artificial Sequence
  Description of Artificial Sequence: Synthetic oligonucleotide
acctgtcaggtgaagttcgctgg

SEQ ID NO: 12 - protein, 118 aa, Artificial Sequence
  Description of Artificial Sequence: Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 13 - protein, 113 aa, Artificial Sequence
  Description of Artificial Sequence: Synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 14 - protein, 19 aa, Artificial Sequence
  Description of Artificial Sequence: Synthetic peptide
GSTSSGSGKPGSGEGSTKG

SEQ ID NO: 15 - protein, 113 aa, Artificial Sequence
  Description of Artificial Sequence: Synthetic polypeptide
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - DNA, 850 bp, Artificial Sequence
  Description of Artificial Sequence: Synthetic polynucleotide
ggtgtcgtgagcggccgctgaactggccaccatggacatgagggtccctgctcagctcctggggctcctgctgctctggctctcaggtgccagatgtgagatcgtgctgacccagagccccggcagcctggccgtgagccccggcaagagggtgaccatgagctgcaagagcagccagagcgtgttcttcagcagcagccagaagaactacctggcctggtaccagcagatccccggccagagccccaggctgctgatctactgggccagcaccagggagagcggcgtgcccgacaggttcaccggcagcggcagcggcagcggcaccgacttcaccctgaccatcagcagcgtgcagcccgaggacctggccatctactactgccaccagtacctgagcagcaggaccttcggccagggcaccaagctggagatcaagaggggcagcaccagcggcagcggcaagcccggcagcggcgagggcagcaccaagggccaggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttccagggcaaggccaccctgaccgccgacaagagcagcaccaccgcctacatgcagctgagcagcctgaccagcgaggacagcgccgtgtactactgcgccagggaggtgaggctgaggtacttcgacgtgtggggccagggcaccaccgtgaccgtgagcagc

SEQ ID NO: 17 - DNA, 695 bp, Artificial Sequence
  Description of Artificial Sequence: Synthetic polynucleotide
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 18 - DNA, 641 bp, Artificial Sequence
  Description of Artificial Sequence: Synthetic polynucleotide
gcggccgcactatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 19 - DNA, 665 bp, Artificial Sequence
  Description of Artificial Sequence: Synthetic polynucleotide
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 20 - protein, 503 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 509 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 552 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 542 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDFWLPIGCAAFVVVCILGCILICWLTKKKYSSSVHDPNGEYMFMRAVNTAKKSRLTDVTLTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 459 aa, Artificial Sequence
  Description of Artificial Sequence: Synthetic polypeptide
MWLQSLLLLGTVACSISIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIGSTSGSGKPGSGEGSTKGLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 398 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRS

SEQ ID NO: 26 - protein, 500 aa, Artificial Sequence
  Description of Artificial Sequence: Synthetic polypeptide
MWLQSLLLLGTVACSISIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIGSTSGSGKPGSGEGSTKGLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 484 aa, Artificial Sequence
  Description of Artificial Sequence: Synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMGERRRGKGHDGLYQGLSTATKDTYDALHMQALPPR

SEQ ID NO: 28 - DNA, 23 bp, Artificial Sequence
  Description of Artificial Sequence: Synthetic oligonucleotide
tggccgggttctagagtgccagg

SEQ ID NO: 29 - DNA, 23 bp, Artificial Sequence
  Description of Artificial Sequence: Synthetic oligonucleotide
ggccgggttctagagtgccaggg

SEQ ID NO: 30 - DNA, 23 bp, Artificial Sequence
  Description of Artificial Sequence: Synthetic oligonucleotide
caccgaggagtgagtagtcctgg

SEQ ID NO: 31 - DNA, 23 bp, Artificial Sequence
  Description of Artificial Sequence: Synthetic oligonucleotide
tccagcgaacttcacctgacagg

SEQ ID NO: 32 - DNA, 23 bp, Artificial Sequence
  Description of Artificial Sequence: Synthetic oligonucleotide
ccaagtatccccggactctttgg

SEQ ID NO: 33 - DNA, 23 bp, Artificial Sequence
  Description of Artificial Sequence: Synthetic oligonucleotide
agcattatccaaagagtccgggg

SEQ ID NO: 34 - DNA, 23 bp, Artificial Sequence
  Description of Artificial Sequence: Synthetic oligonucleotide
acttgggtggaagtattgtctgg

SEQ ID NO: 35 - DNA, 23 bp, Artificial Sequence
  Description of Artificial Sequence: Synthetic oligonucleotide
gtctgcgagtctgcgtgcgtggg

SEQ ID NO: 36 - DNA, 23 bp, Artificial Sequence
  Description of Artificial Sequence: Synthetic oligonucleotide
cgtctgcgagtctgcgtgcgtgg

SEQ ID NO: 37 - DNA, 23 bp, Artificial Sequence
  Description of Artificial Sequence: Synthetic oligonucleotide
gcgagtctgcgtgcgtgggaagg

SEQ ID NO: 38 - DNA, 1568 bp, Artificial Sequence
  Description of Artificial Sequence: Synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggcctggatatctacatctgggccccactggccggcacctgtggcgtgctgctgctgtctctcgtgatcaccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggaatgaagggcgagcggagaagaggcaagggccacgatggactgtaccagggcctgagcaccgccaccaaggacacctatgacgccctgcacatgcaggccctgccccccagatgaaattcatcgacgttaactattctag

SEQ ID NO: 39 - DNA, 1586 bp, Artificial Sequence
  Description of Artificial Sequence: Synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagagaagaggccgggaccctgagatgggcggcaagcccagacggaagaaccctcaggaaggcctgtataacgaactgcagaaagacaagatggccgaggcctactccgagatcggcatgaagggcgaacggcggagaggcaagggacacgatggactgtaccagggcctgagcaccgccaccaaggacacctatgacgccctgcacatgcaggccctgccccccagatgaaattcatcgacgttaactattctag

SEQ ID NO: 40 - DNA, 1715 bp, Artificial Sequence
  Description of Artificial Sequence: Synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctacagatccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagagaagaggccgggaccctgagatgggcggcaagcccagacggaagaaccctcaggaaggcctgtataacgaactgcagaaagacaagatggccgaggcctactccgagatcggaatgaagggcgagcggcggagaggcaagggacacgatggactgtaccagggcctgagcaccgccaccaaggacacctatgacgccctgcacatgcaggccctgccccccagatgaaattcatcgacgttaactattctag

SEQ ID NO: 41 - DNA, 1436 bp, Artificial Sequence
  Description of Artificial Sequence: Synthetic polynucleotide
ggtgtcgtgagcggccgctgaactggccaccatgtggctgcagtctctgctgctgctgggcaccgtggcctgcagcatcagcatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgccctctagattttccggcagcggctccggcaccgactacagcctgaccatctccaacctggaacaggaagatatcgctacctacttctgtcagcaaggcaacaccctgccctacaccttcggcggaggcaccaagctggaaatcggcagcacaagcggctctggcaagcctggatctggcgagggctctaccaagggcctgcaggaatctggccctggactggtggcccctagccagagcctgtctgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttatagcctgctcgtgacagtggccttcatcatcttttgggtgcgcgtgaagttcagccgcagcgccgatgcccctgcctatcagcagggacagaaccagctgtacaacgagctgaacctgggcagacgggaagagtacgacgtgctggacaagagaagaggccgggaccctgagatgggcggcaagcccagaagaaagaacccccaggaaggcctgtataacgaactgcagaaagacaagatggccgaggcctacagcgagatcggcatgaagggcgaacggcggagaggcaagggccacgatggactgtatcagggcctgagcaccgccaccaaggacacctatgacgccctgcacatgcaggctctgccccctcgctgaaattcatcgacgttaactattctag

SEQ ID NO: 42 - DNA, 1253 bp, Artificial Sequence
  Description of Artificial Sequence: Synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctacagaagctgaaattcatcgacgttaactattctag

SEQ ID NO: 43 - DNA, 1559 bp, Artificial Sequence
  Description of Artificial Sequence: Synthetic polynucleotide
ggtgtcgtgagcggccgctgaactggccaccatgtggctgcagtctctgctgctgctgggcaccgtggcctgcagcatcagcatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgccctctagattttccggcagcggctccggcaccgactacagcctgaccatctccaacctggaacaggaagatatcgctacctacttctgtcagcaaggcaacaccctgccctacaccttcggcggaggcaccaagctggaaatcggcagcacaagcggctctggcaagcctggatctggcgagggctctaccaagggcctgcaggaatctggccctggactggtggcccctagccagagcctgtctgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttatagcctgctcgtgacagtggccttcatcatcttttgggtgcgcagcaagcggagccggctgctgcactccgactacatgaacatgacccccagacggccaggccccacccggaaacactatcagccttacgcccctcccagagacttcgccgcctaccggtccagagtgaagttcagcagatccgccgacgcccctgcctatcagcagggacagaaccagctgtacaacgagctgaacctgggcagacgggaagagtacgacgtgctggacaagagaagaggccgggaccctgagatgggcggcaagcccagaagaaagaacccccaggaaggcctgtataacgaactgcagaaagacaagatggccgaggcctacagcgagatcggcatgaagggcgaacggcggagaggcaagggccacgatggactgtatcagggcctgagcaccgccaccaaggacacctatgacgccctgcacatgcaggctctgccccctcgctgaaattcatcgacgttaactattctag

SEQ ID NO: 44 - DNA, 1514 bp, Artificial Sequence
  Description of Artificial Sequence: Synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtccagcatcgaagtgatgtacccccctccctacctggacaacgagaagtccaacggcaccatcatccacgtgaagggcaagcacctgtgccccagccctctgtttcctggccctagcaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggcatgaagggcgagcggagaagaggcaagggccacgatggactgtaccagggcctgagcaccgccaccaaggacacctatgacgccctgcacatgcaggccctgccccccagatgaaattcatcgacgttaactattctag

SEQ ID NO: 45 - DNA, 21 bp, Unknown
  Description of Unknown: Cell-surface antigen sequence
  modified_base (19)..(19): a, c, t, g, unknown or other
atcgatcgatcgtacgcgngg

SEQ ID NO: 46 - DNA, 22 bp, Unknown
  Description of Unknown: Cell-surface antigen sequence
  modified_base (20)..(20): a, c, t, g, unknown or other
atgcatcgtacatcgatcgngg

SEQ ID NO: 47 - protein, 38 aa, Artificial Sequence
  Description of Artificial Sequence: Synthetic polypeptide
CWLTKKKYSSSVHDPNGEYMFMRAVNTAKKSRLTDVTL

SEQ ID NO: 48 - DNA, 23 bp, Artificial Sequence
  Description of Artificial Sequence: Synthetic oligonucleotide
cctcactagacttgacccacagg

SEQ ID NO: 49 - DNA, 23 bp, Artificial Sequence
  Description of Artificial Sequence: Synthetic oligonucleotide
atccctggcactctagaacccgg

SEQ ID NO: 50 - DNA, 20 bp, Artificial Sequence
  Description of Artificial Sequence: Synthetic oligonucleotide
atccctggcactctagaacc

SEQ ID NO: 51 - DNA, 20 bp, Artificial Sequence
  Description of Artificial Sequence: Synthetic oligonucleotide
ggccgggttctagagtgcca

SEQ ID NO: 52 - DNA, 31 bp, Homo sapiens
ttctcctcactagacttgacccacaggccca

SEQ ID NO: 53 - DNA, 31 bp, Homo sapiens
tgggcctgtgggtcaagtctagtgaggagaa

SEQ ID NO: 54 - DNA, 37 bp, Homo sapiens
cctgtgggtcaagtctagtgaggagaaagaggggatg

SEQ ID NO: 55 - DNA, 38 bp, Artificial Sequence
  Description of Artificial Sequence: Synthetic oligonucleotide
cctgtgggtcaagtctaggggggagaaagaggggatgc

SEQ ID NO: 56 - DNA, 31 bp, Homo sapiens
tcatccctggcactctagaacccggccactc

SEQ ID NO: 57 - DNA, 31 bp, Homo sapiens
gagtggccgggttctagagtgccagggatga

SEQ ID NO: 58 - DNA, 20 bp, Artificial Sequence
  Description of Artificial Sequence: Synthetic oligonucleotide
cctcactagacttgacccac

SEQ ID NO: 59 - DNA, 31 bp, Homo sapiens
gtggccgggttctagagtgccagggatgagg

SEQ ID NO: 60 - RNA, 23 nt, Artificial Sequence
  Description of Artificial Sequence: Synthetic oligonucleotide
accugucaggugaaguucgcugg

SEQ ID NO: 61 - RNA, 23 nt, Artificial Sequence
  Description of Artificial Sequence: Synthetic oligonucleotide
uggccggguucuagagugccagg

SEQ ID NO: 62 - RNA, 23 nt, Artificial Sequence
  Description of Artificial Sequence: Synthetic oligonucleotide
ggccggguucuagagugccaggg

SEQ ID NO: 63 - RNA, 23 nt, Artificial Sequence
  Description of Artificial Sequence: Synthetic oligonucleotide
caccgaggagugaguaguccugg

SEQ ID NO: 64 - RNA, 23 nt, Artificial Sequence
  Description of Artificial Sequence: Synthetic oligonucleotide
uccagcgaacuucaccugacagg

SEQ ID NO: 65 - RNA, 23 nt, Artificial Sequence
  Description of Artificial Sequence: Synthetic oligonucleotide
ccucacuagacuugacccacagg

SEQ ID NO: 66 - RNA, 23 nt, Artificial Sequence
  Description of Artificial Sequence: Synthetic oligonucleotide
aucccuggcacucuagaacccgg

SEQ ID NO: 67 - RNA, 20 nt, Artificial Sequence
  Description of Artificial Sequence: Synthetic oligonucleotide
aucccuggcacucuagaacc

SEQ ID NO: 68 - RNA, 20 nt, Artificial Sequence
  Description of Artificial Sequence: Synthetic oligonucleotide
ggccggguucuagagugcca

SEQ ID NO: 69 - skipped

SEQ ID NO: 70 - RNA, 20 nt, Artificial Sequence
  Description of Artificial Sequence: Synthetic oligonucleotide
ccucacuagacuugacccac

SEQ ID NO: 71 - RNA, 28 nt, Artificial Sequence
  Description of Artificial Sequence: Synthetic oligonucleotide
ccucaucccuggcacucuagaacccggc

SEQ ID NO: 72 - RNA, 28 nt, Artificial Sequence
  Description of Artificial Sequence: Synthetic oligonucleotide
gaguggccggguucuagagugccaggga

SEQ ID NO: 73 - RNA, 28 nt, Artificial Sequence
  Description of Artificial Sequence: Synthetic oligonucleotide
uucuccucacuagacuugacccacaggc

SEQ ID NO: 74 - protein, 156 aa, Artificial Sequence
  Description of Artificial Sequence: Synthetic polypeptide
  MISC_FEATURE (1)..(12): This region may encompass 3-12 residues
  MISC_FEATURE (14)..(25): This region may encompass 3-12 residues
  MISC_FEATURE (27)..(38): This region may encompass 3-12 residues
  MISC_FEATURE (40)..(51): This region may encompass 3-12 residues
  MISC_FEATURE (53)..(64): This region may encompass 3-12 residues
  MISC_FEATURE (66)..(77): This region may encompass 3-12 residues
  MISC_FEATURE (79)..(90): This region may encompass 3-12 residues
  MISC_FEATURE (92)..(103): This region may encompass 3-12 residues
  MISC_FEATURE (105)..(116): This region may encompass 3-12 residues
  MISC_FEATURE (118)..(129): This region may encompass 3-12 residues
  MISC_FEATURE (131)..(142): This region may encompass 3-12 residues
  MISC_FEATURE (144)..(155): This region may encompass 3-12 residues
  MISC_FEATURE (1)..(156): This sequence may encompass 3-12 “GlyxSer” repeating units, wherein x = 3-12
  See specification as filed for detailed description of substitutions and preferred embodiments
GGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGSGGGGGGGGGGGGS

SEQ ID NO: 75 - DNA, 40 bp, Homo sapiens
agtggccgggttctagagtgccagggatgaggattttggg

SEQ ID NO: 76 - DNA, 40 bp, Artificial Sequence
  Description of Artificial Sequence: Synthetic oligonucleotide
agtggccgggttctagagtgccggggatgaggattttggg

SEQ ID NO: 77 - DNA, 42 bp, Homo sapiens
ctcatccctggcactctagaacccggccactccaaaaacctg

SEQ ID NO: 78 - DNA, 42 bp, Artificial Sequence; identical to SEQ ID NO: 77
  Description of Artificial Sequence: Synthetic oligonucleotide

SEQ ID NO: 79 - DNA, 20 bp, Homo sapiens
atccctggcactcaggagcc

SEQ ID NO: 80 - DNA, 20 bp, Homo sapiens
aaccctggctctctaaaacc

SEQ ID NO: 81 - DNA, 20 bp, Homo sapiens
atccctggcaccctggcacc

SEQ ID NO: 82 - DNA, 20 bp, Homo sapiens
atccctggcactccagggcc

SEQ ID NO: 83 - DNA, 20 bp, Homo sapiens
agccctggcagtctggaacc

SEQ ID NO: 84 - DNA, 20 bp, Homo sapiens
atcccaggccctctataacc

SEQ ID NO: 85 - DNA, 20 bp, Homo sapiens
atccatggtactctggaacc

SEQ ID NO: 86 - DNA, 20 bp, Homo sapiens
aacccaggcactctagcacc

SEQ ID NO: 87 - DNA, 20 bp, Homo sapiens
acccctagcactctagagcc

SEQ ID NO: 88 - DNA, 20 bp, Homo sapiens
acccctggcagtctagagcc

SEQ ID NO: 89 - DNA, 20 bp, Homo sapiens
atccctgacactctgggacc

SEQ ID NO: 90 - DNA, 20 bp, Homo sapiens
agccctcgcactctggaacc

SEQ ID NO: 91 - DNA, 20 bp, Homo sapiens
atctctggcagactagaacc

SEQ ID NO: 92 - DNA, 20 bp, Homo sapiens
acccctgacactctagaaag

SEQ ID NO: 93 - DNA, 20 bp, Homo sapiens
atccctggcacactgggccc

SEQ ID NO: 94 - DNA, 20 bp, Homo sapiens
atcactgtctccctagaacc

SEQ ID NO: 95 - DNA, 20 bp, Homo sapiens
atccctgaccctccagaaca

SEQ ID NO: 96 - DNA, 20 bp, Homo sapiens
acccctggctccctagagcc

SEQ ID NO: 97 - DNA, 20 bp, Homo sapiens
ctgcctggctctctagcacc

SEQ ID NO: 98 - DNA, 20 bp, Homo sapiens
gtctctggcactctaggccc

SEQ ID NO: 99 - DNA, 20 bp, Homo sapiens
cctcacaagattagacccac

SEQ ID NO: 100 - DNA, 20 bp, Homo sapiens
tctcactggccttgacccac

SEQ ID NO: 101 - DNA, 20 bp, Homo sapiens
cctcactggacttgactcag

SEQ ID NO: 102 - DNA, 20 bp, Homo sapiens
cctcactggacaagacccac

SEQ ID NO: 103 - DNA, 20 bp, Homo sapiens
cctcactagactagatccat

SEQ ID NO: 104 - DNA, 20 bp, Homo sapiens
cctcgctagcctttacccac

SEQ ID NO: 105 - DNA, 20 bp, Homo sapiens
cctcacaagacaagacccac

SEQ ID NO: 106 - DNA, 20 bp, Homo sapiens
cttcaccagccttgacccac

SEQ ID NO: 107 - DNA, 20 bp, Homo sapiens
cctcaccagccctgacccac

SEQ ID NO: 108 - DNA, 20 bp, Homo sapiens
tcacactggccttgacccac

SEQ ID NO: 109 - DNA, 20 bp, Homo sapiens
ccttactagcctccacccac

SEQ ID NO: 110 - DNA, 20 bp, Homo sapiens
cctcacaagataagacccac

SEQ ID NO: 111 - DNA, 20 bp, Homo sapiens
cctaactagaattgccacac

SEQ ID NO: 112 - DNA, 20 bp, Homo sapiens
cctctatatacttgtcccac

SEQ ID NO: 113 - DNA, 31 bp, Homo sapiens
cctcatccctggcactctagaacccggccac

SEQ ID NO: 114 - DNA, 57 bp, Homo sapiens
actagacttgacccacaggcccaaaatcctcatccctggcactctagaacccggcca

SEQ ID NO: 115 - RNA, 20 nt, Artificial Sequence
  Synthetic oligonucleotide
guuguagagaaauauuucuc

SEQ ID NO: 116 - RNA, 20 nt, Artificial Sequence
  Synthetic oligonucleotide
ggagagguuccugaucuugu

SEQ ID NO: 117 - DNA, 20 bp, Homo sapiens
gttgtagagaaatatttctc

SEQ ID NO: 118 - DNA, 20 bp, Homo sapiens
ggagaggttcctgatcttgt

SEQ ID NO: 119 - RNA, 20 nt, Artificial Sequence
  Synthetic oligonucleotide
ugaauaucuccaacaagauc